SEQUENCE LISTING (88 sequences)

SEQ ID NO: 1 - RNA, 100 nt, synthetic construct
  source 1..100: mol_type = other RNA
gagaatcaaaatcggtgaatgttttagagctagaaatagcaagttaaaataaggctagtccgttatcaacttgaaaaagtggcaccgagtcggtgctttt

SEQ ID NO: 2 - RNA, 100 nt, synthetic construct
  source 1..100: mol_type = other RNA
cacccagatcgtcagcgccggttttagagctagaaatagcaagttaaaataaggctagtccgttatcaacttgaaaaagtggcaccgagtcggtgctttt

SEQ ID NO: 3 - protein, 9 aa, Homo sapiens
VLHDDLLEA

SEQ ID NO: 4 - protein, 9 aa, Homo sapiens
VLRDDLLEA

SEQ ID NO: 5 - protein, 9 aa, Homo sapiens
YIGEVLVSV

SEQ ID NO: 6 - protein, 9 aa, Homo sapiens
YIGEVLVSM

SEQ ID NO: 7 - DNA, 399 bp, synthetic construct
  source 1..399: mol_type = other DNA
atgggcacaagcctgctgtgttggatggccctgtgtctgctgggagccgatcatgccgatacgggagtgtcccaggatcctcggcacaagattaccaagcggggccagaacgtgaccttcagatgcgaccctatcagcgagcacaaccggctgtactggtacagacagacactcggccagggacctgagttcctgacctacttccagaacgaggcccaactcgaaaagagtagactgctgagcgacagattcagcgccgaaagacccaagggcagcttcagcaccctggaaatccagagaaccgagcagggcgacagcgccatgtatctgtgtgccagctctctggtgtccggcaacgagcagtttttcggccctggcaccagactgaccgtgctggaa

SEQ ID NO: 8 - protein, 133 aa, synthetic construct
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSLVSGNEQFFGPGTRLTVLE

SEQ ID NO: 9 - DNA, 534 bp, synthetic construct
  source 1..534: mol_type = other DNA
gatctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtcagtactgatcctcagcctctcaaagaacagcccgctctgaacgacagccggtactgtctgagcagcagactgagagtgtccgccaccttctggcagaatcctagaaatcatttcaggtgccaggtgcaattctatggcctgagcgaaaacgatgaatggacccaggatagagccaagcctgtgacacaaattgtgagcgctgaggcatggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggatagcagaggc

SEQ ID NO: 10 - protein, 178 aa, Homo sp.
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 11 - DNA, 933 bp, synthetic construct
  source 1..933: mol_type = other DNA
atgggcacaagcctgctgtgttggatggccctgtgtctgctgggagccgatcatgccgatacgggagtgtcccaggatcctcggcacaagattaccaagcggggccagaacgtgaccttcagatgcgaccctatcagcgagcacaaccggctgtactggtacagacagacactcggccagggacctgagttcctgacctacttccagaacgaggcccaactcgaaaagagtagactgctgagcgacagattcagcgccgaaagacccaagggcagcttcagcaccctggaaatccagagaaccgagcagggcgacagcgccatgtatctgtgtgccagctctctggtgtccggcaacgagcagtttttcggccctggcaccagactgaccgtgctggaagatctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtcagtactgatcctcagcctctcaaagaacagcccgctctgaacgacagccggtactgtctgagcagcagactgagagtgtccgccaccttctggcagaatcctagaaatcatttcaggtgccaggtgcaattctatggcctgagcgaaaacgatgaatggacccaggatagagccaagcctgtgacacaaattgtgagcgctgaggcatggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggatagcagaggc

SEQ ID NO: 12 - protein, 311 aa, synthetic construct
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSLVSGNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 13 - DNA, 405 bp, synthetic construct
  source 1..405: mol_type = other DNA
atggccagcgctcctattagcatgctggccatgctgttcaccctgagcggactgagagcacagtctgtggcccagcctgaggaccaagtgaatgtggccgagggcaatcccctgaccgtgaagtgtacctactccgtgtctggcaacccctacctgttttggtacgtgcagtaccccaaccggggcctgcagttcctgctgaagtacatcaccggcgacaacctcgtgaagggctcctatggcttcgaggccgaattcaacaagagccagaccagcttccacctgaagaaacccagcgcactcgtgtccgatagcgccctctacttttgcgccgtgcgggctcctacaagcggcacctacaagtacatcttcggcaccggcacacggctgaaggtgctggccaac

SEQ ID NO: 14 - protein, 135 aa, synthetic construct
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRAPTSGTYKYIFGTGTRLKVLAN

SEQ ID NO: 15 - DNA, 420 bp, synthetic construct
  source 1..420: mol_type = other DNA
attcagaatcccgatccagctgtctatcaacttcgcgatagcaagagcagcgacaagagcgtgtgtctgtttacagacttcgactcccagaccaacgtgtcccagagcaaggacagcgacgtgtacatcacagacaagacagtgctggacatgcggagcatggacttcaagagcaacagcgccgtggcctggtccaacaagtccgatttcgcctgcgccaacgccttcaacaacagcattatccccgaggacacattcttcccaagtcctgagagcagctgcgacgtgaagctggtggaaaagtccttcgaaaccgacaccaacctgaatttccagaacctgagcgtgatcggcttcagaatcctgctgctgaaagtggccggcttcaacctgctgatgaccctgagactgtggtctagc

SEQ ID NO: 16 - protein, 140 aa, Homo sapiens
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 17 - DNA, 825 bp, synthetic construct
  source 1..825: mol_type = other DNA
atggccagcgctcctattagcatgctggccatgctgttcaccctgagcggactgagagcacagtctgtggcccagcctgaggaccaagtgaatgtggccgagggcaatcccctgaccgtgaagtgtacctactccgtgtctggcaacccctacctgttttggtacgtgcagtaccccaaccggggcctgcagttcctgctgaagtacatcaccggcgacaacctcgtgaagggctcctatggcttcgaggccgaattcaacaagagccagaccagcttccacctgaagaaacccagcgcactcgtgtccgatagcgccctctacttttgcgccgtgcgggctcctacaagcggcacctacaagtacatcttcggcaccggcacacggctgaaggtgctggccaacattcagaatcccgatccagctgtctatcaacttcgcgatagcaagagcagcgacaagagcgtgtgtctgtttacagacttcgactcccagaccaacgtgtcccagagcaaggacagcgacgtgtacatcacagacaagacagtgctggacatgcggagcatggacttcaagagcaacagcgccgtggcctggtccaacaagtccgatttcgcctgcgccaacgccttcaacaacagcattatccccgaggacacattcttcccaagtcctgagagcagctgcgacgtgaagctggtggaaaagtccttcgaaaccgacaccaacctgaatttccagaacctgagcgtgatcggcttcagaatcctgctgctgaaagtggccggcttcaacctgctgatgaccctgagactgtggtctagc

SEQ ID NO: 18 - protein, 275 aa, unidentified
MASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRAPTSGTYKYIFGTGTRLKVLANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 19 - DNA, 1824 bp, synthetic construct
  source 1..1824: mol_type = other DNA
atgggcacaagcctgctgtgttggatggccctgtgtctgctgggagccgatcatgccgatacgggagtgtcccaggatcctcggcacaagattaccaagcggggccagaacgtgaccttcagatgcgaccctatcagcgagcacaaccggctgtactggtacagacagacactcggccagggacctgagttcctgacctacttccagaacgaggcccaactcgaaaagagtagactgctgagcgacagattcagcgccgaaagacccaagggcagcttcagcaccctggaaatccagagaaccgagcagggcgacagcgccatgtatctgtgtgccagctctctggtgtccggcaacgagcagtttttcggccctggcaccagactgaccgtgctggaagatctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtcagtactgatcctcagcctctcaaagaacagcccgctctgaacgacagccggtactgtctgagcagcagactgagagtgtccgccaccttctggcagaatcctagaaatcatttcaggtgccaggtgcaattctatggcctgagcgaaaacgatgaatggacccaggatagagccaagcctgtgacacaaattgtgagcgctgaggcatggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggatagcagaggcggaagcggcgccacaaacttcagcctgcttaaacaggccggcgacgtggaagagaaccccggacctatggccagcgctcctattagcatgctggccatgctgttcaccctgagcggactgagagcacagtctgtggcccagcctgaggaccaagtgaatgtggccgagggcaatcccctgaccgtgaagtgtacctactccgtgtctggcaacccctacctgttttggtacgtgcagtaccccaaccggggcctgcagttcctgctgaagtacatcaccggcgacaacctcgtgaagggctcctatggcttcgaggccgaattcaacaagagccagaccagcttccacctgaagaaacccagcgcactcgtgtccgatagcgccctctacttttgcgccgtgcgggctcctacaagcggcacctacaagtacatcttcggcaccggcacacggctgaaggtgctggccaacattcagaatcccgatccagctgtctatcaacttcgcgatagcaagagcagcgacaagagcgtgtgtctgtttacagacttcgactcccagaccaacgtgtcccagagcaaggacagcgacgtgtacatcacagacaagacagtgctggacatgcggagcatggacttcaagagcaacagcgccgtggcctggtccaacaagtccgatttcgcctgcgccaacgccttcaacaacagcattatccccgaggacacattcttcccaagtcctgagagcagctgcgacgtgaagctggtggaaaagtccttcgaaaccgacaccaacctgaatttccagaacctgagcgtgatcggcttcagaatcctgctgctgaaagtggccggcttcaacctgctgatgaccctgagactgtggtctagc

SEQ ID NO: 20 - protein, 608 aa, synthetic construct
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSLVSGNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRAPTSGTYKYIFGTGTRLKVLANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 21 - DNA, 2361 bp, synthetic construct
  source 1..2361: mol_type = other DNA
atgggcacaagcctgctgtgttggatggccctgtgtctgctgggagccgatcatgccgatacgggagtgtcccaggatcctcggcacaagattaccaagcggggccagaacgtgaccttcagatgcgaccctatcagcgagcacaaccggctgtactggtacagacagacactcggccagggacctgagttcctgacctacttccagaacgaggcccaactcgaaaagagtagactgctgagcgacagattcagcgccgaaagacccaagggcagcttcagcaccctggaaatccagagaaccgagcagggcgacagcgccatgtatctgtgtgccagctctctggtgtccggcaacgagcagtttttcggccctggcaccagactgaccgtgctggaagatctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtcagtactgatcctcagcctctcaaagaacagcccgctctgaacgacagccggtactgtctgagcagcagactgagagtgtccgccaccttctggcagaatcctagaaatcatttcaggtgccaggtgcaattctatggcctgagcgaaaacgatgaatggacccaggatagagccaagcctgtgacacaaattgtgagcgctgaggcatggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggatagcagaggcggaagcggcgccacaaacttcagcctgcttaaacaggccggcgacgtggaagagaaccccggacctatggccagcgctcctattagcatgctggccatgctgttcaccctgagcggactgagagcacagtctgtggcccagcctgaggaccaagtgaatgtggccgagggcaatcccctgaccgtgaagtgtacctactccgtgtctggcaacccctacctgttttggtacgtgcagtaccccaaccggggcctgcagttcctgctgaagtacatcaccggcgacaacctcgtgaagggctcctatggcttcgaggccgaattcaacaagagccagaccagcttccacctgaagaaacccagcgcactcgtgtccgatagcgccctctacttttgcgccgtgcgggctcctacaagcggcacctacaagtacatcttcggcaccggcacacggctgaaggtgctggccaacattcagaatcccgatccagctgtctatcaacttcgcgatagcaagagcagcgacaagagcgtgtgtctgtttacagacttcgactcccagaccaacgtgtcccagagcaaggacagcgacgtgtacatcacagacaagacagtgctggacatgcggagcatggacttcaagagcaacagcgccgtggcctggtccaacaagtccgatttcgcctgcgccaacgccttcaacaacagcattatccccgaggacacattcttcccaagtcctgagagcagctgcgacgtgaagctggtggaaaagtccttcgaaaccgacaccaacctgaatttccagaacctgagcgtgatcggcttcagaatcctgctgctgaaagtggccggcttcaacctgctgatgaccctgagactgtggtctagcggctctggcgaaggtagaggcagcctccttacatgcggcgacgtcgaagaaaatcctggtccaatgggcacctctctgctctgctggatggctctttgcctcctgggtgctgaccacgccgatacagcctgtccttacagcaaccctagcctgtgttctggcggcggaggatctgaactgcctacacagggcaccttctccaatgtgtccacaaatgtgtctcccgccaagcctaccaccaccgcttgtccctactccaatccatctctgtgcagcggaggcggcggatctcctgctcctagacctcctacaccagctcctacaatcgccagccagccactgtctctgaggccagaagcttgtagaccagctgctggcggagccgtgcatacaagaggactggactttgcctgcgacatctacatctgggcccctctggctggaacatgtggcgttctgctgctgtccctggtcatcaccctgtactgcaaccaccggaacaggcggagagtgtgcaagtgccctagacctgtggtt

SEQ ID NO: 22 - protein, 787 aa, synthetic construct
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSLVSGNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMASAPISMLAMLFTLSGLRAQSVAQPEDQVNVAEGNPLTVKCTYSVSGNPYLFWYVQYPNRGLQFLLKYITGDNLVKGSYGFEAEFNKSQTSFHLKKPSALVSDSALYFCAVRAPTSGTYKYIFGTGTRLKVLANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGEGRGSLLTCGDVEENPGPMGTSLLCWMALCLLGADHADTACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 23 - DNA, 2923 bp, synthetic construct
  source 1..2923: mol_type = other DNA
cgattagtccaatttgttaaagacaggatatcagtggtccaggctctagttttgactcaacaatatcaccagctgaagcctatagagtacgagccatagatagaataaaagattttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgatctagatctcgaatctctagagccgccaccatgggcacaagcctgctgtgttggatggccctgtgtctgctgggagccgatcatgccgatacgggagtgtcccaggatcctcggcacaagattaccaagcggggccagaacgtgaccttcagatgcgaccctatcagcgagcacaaccggctgtactggtacagacagacactcggccagggacctgagttcctgacctacttccagaacgaggcccaactcgaaaagagtagactgctgagcgacagattcagcgccgaaagacccaagggcagcttcagcaccctggaaatccagagaaccgagcagggcgacagcgccatgtatctgtgtgccagctctctggtgtccggcaacgagcagtttttcggccctggcaccagactgaccgtgctggaagatctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtcagtactgatcctcagcctctcaaagaacagcccgctctgaacgacagccggtactgtctgagcagcagactgagagtgtccgccaccttctggcagaatcctagaaatcatttcaggtgccaggtgcaattctatggcctgagcgaaaacgatgaatggacccaggatagagccaagcctgtgacacaaattgtgagcgctgaggcatggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggatagcagaggcggaagcggcgccacaaacttcagcctgcttaaacaggccggcgacgtggaagagaaccccggacctatggccagcgctcctattagcatgctggccatgctgttcaccctgagcggactgagagcacagtctgtggcccagcctgaggaccaagtgaatgtggccgagggcaatcccctgaccgtgaagtgtacctactccgtgtctggcaacccctacctgttttggtacgtgcagtaccccaaccggggcctgcagttcctgctgaagtacatcaccggcgacaacctcgtgaagggctcctatggcttcgaggccgaattcaacaagagccagaccagcttccacctgaagaaacccagcgcactcgtgtccgatagcgccctctacttttgcgccgtgcgggctcctacaagcggcacctacaagtacatcttcggcaccggcacacggctgaaggtgctggccaacattcagaatcccgatccagctgtctatcaacttcgcgatagcaagagcagcgacaagagcgtgtgtctgtttacagacttcgactcccagaccaacgtgtcccagagcaaggacagcgacgtgtacatcacagacaagacagtgctggacatgcggagcatggacttcaagagcaacagcgccgtggcctggtccaacaagtccgatttcgcctgcgccaacgccttcaacaacagcattatccccgaggacacattcttcccaagtcctgagagcagctgcgacgtgaagctggtggaaaagtccttcgaaaccgacaccaacctgaatttccagaacctgagcgtgatcggcttcagaatcctgctgctgaaagtggccggcttcaacctgctgatgaccctgagactgtggtctagcggctctggcgaaggtagaggcagcctccttacatgcggcgacgtcgaagaaaatcctggtccaatgggcacctctctgctctgctggatggctctttgcctcctgggtgctgaccacgccgatacagcctgtccttacagcaaccctagcctgtgttctggcggcggaggatctgaactgcctacacagggcaccttctccaatgtgtccacaaatgtgtctcccgccaagcctaccaccaccgcttgtccctactccaatccatctctgtgcagcggaggcggcggatctcctgctcctagacctcctacaccagctcctacaatcgccagccagccactgtctctgaggccagaagcttgtagaccagctgctggcggagccgtgcatacaagaggactggactttgcctgcgacatctacatctgggcccctctggctggaacatgtggcgttctgctgctgtccctggtcatcaccctgtactgcaaccaccggaacaggcggagagtgtgcaagtgccctagacctgtggtt

SEQ ID NO: 24 - DNA, 9246 bp, synthetic construct
  source 1..9246: mol_type = other DNA
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgattagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggctgcattgatcacgtgaggggcgattagtccaatttgttaaagacaggatatcagtggtccaggctctagttttgactcaacaatatcaccagctgaagcctatagagtacgagccatagatagaataaaagattttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgatctagatctcgaatctctagagccgccaccatgggcacaagcctgctgtgttggatggccctgtgtctgctgggagccgatcatgccgatacgggagtgtcccaggatcctcggcacaagattaccaagcggggccagaacgtgaccttcagatgcgaccctatcagcgagcacaaccggctgtactggtacagacagacactcggccagggacctgagttcctgacctacttccagaacgaggcccaactcgaaaagagtagactgctgagcgacagattcagcgccgaaagacccaagggcagcttcagcaccctggaaatccagagaaccgagcagggcgacagcgccatgtatctgtgtgccagctctctggtgtccggcaacgagcagtttttcggccctggcaccagactgaccgtgctggaagatctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtcagtactgatcctcagcctctcaaagaacagcccgctctgaacgacagccggtactgtctgagcagcagactgagagtgtccgccaccttctggcagaatcctagaaatcatttcaggtgccaggtgcaattctatggcctgagcgaaaacgatgaatggacccaggatagagccaagcctgtgacacaaattgtgagcgctgaggcatggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggatagcagaggcggaagcggcgccacaaacttcagcctgcttaaacaggccggcgacgtggaagagaaccccggacctatggccagcgctcctattagcatgctggccatgctgttcaccctgagcggactgagagcacagtctgtggcccagcctgaggaccaagtgaatgtggccgagggcaatcccctgaccgtgaagtgtacctactccgtgtctggcaacccctacctgttttggtacgtgcagtaccccaaccggggcctgcagttcctgctgaagtacatcaccggcgacaacctcgtgaagggctcctatggcttcgaggccgaattcaacaagagccagaccagcttccacctgaagaaacccagcgcactcgtgtccgatagcgccctctacttttgcgccgtgcgggctcctacaagcggcacctacaagtacatcttcggcaccggcacacggctgaaggtgctggccaacattcagaatcccgatccagctgtctatcaacttcgcgatagcaagagcagcgacaagagcgtgtgtctgtttacagacttcgactcccagaccaacgtgtcccagagcaaggacagcgacgtgtacatcacagacaagacagtgctggacatgcggagcatggacttcaagagcaacagcgccgtggcctggtccaacaagtccgatttcgcctgcgccaacgccttcaacaacagcattatccccgaggacacattcttcccaagtcctgagagcagctgcgacgtgaagctggtggaaaagtccttcgaaaccgacaccaacctgaatttccagaacctgagcgtgatcggcttcagaatcctgctgctgaaagtggccggcttcaacctgctgatgaccctgagactgtggtctagcggctctggcgaaggtagaggcagcctccttacatgcggcgacgtcgaagaaaatcctggtccaatgggcacctctctgctctgctggatggctctttgcctcctgggtgctgaccacgccgatacagcctgtccttacagcaaccctagcctgtgttctggcggcggaggatctgaactgcctacacagggcaccttctccaatgtgtccacaaatgtgtctcccgccaagcctaccaccaccgcttgtccctactccaatccatctctgtgcagcggaggcggcggatctcctgctcctagacctcctacaccagctcctacaatcgccagccagccactgtctctgaggccagaagcttgtagaccagctgctggcggagccgtgcatacaagaggactggactttgcctgcgacatctacatctgggcccctctggctggaacatgtggcgttctgctgctgtccctggtcatcaccctgtactgcaaccaccggaacaggcggagagtgtgcaagtgccctagacctgtggtttaagtaggtgagttcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacgtcaaaagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttcgctgaaagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaac

SEQ ID NO: 25 - protein, 136 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPKNYGGSQGNLIFGKGTKLSVKPN

SEQ ID NO: 26 - protein, 276 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPKNYGGSQGNLIFGKGTKLSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 27 - protein, 135 aa, synthetic construct
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSFPGLASYEQYFGPGTRLTVTE

SEQ ID NO: 28 - protein, 313 aa, synthetic construct
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSFPGLASYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 29 - protein, 135 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRQGYGGSQGNLIFGKGTKLSVKPN

SEQ ID NO: 30 - protein, 275 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRQGYGGSQGNLIFGKGTKLSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 31 - protein, 135 aa, synthetic construct
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLIQGTNSPLHFGNGTRLTVTE

SEQ ID NO: 32 - protein, 311 aa, synthetic construct
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLIQGTNSPLHFGNGTRLTVTEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 33 - protein, 136 aa, synthetic construct
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVQAWDYGGSQGNLIFGKGTKLSVKPN

SEQ ID NO: 34 - protein, 276 aa, synthetic construct
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVQAWDYGGSQGNLIFGKGTKLSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 35 - protein, 134 aa, synthetic construct
MDTRVLCCAVICLLGAGLSNAGVMQNPRHLVRRRGQEARLRCSPMKGHSHVYWYRQLPEEGLKFMVYLQKENIIDESGMPKERFSAEFPKEGPSILRIQQVVRGDSAAYFCASSTREGTDTQYFGPGTRLTVLE

SEQ ID NO: 36 - protein, 312 aa, synthetic construct
MDTRVLCCAVICLLGAGLSNAGVMQNPRHLVRRRGQEARLRCSPMKGHSHVYWYRQLPEEGLKFMVYLQKENIIDESGMPKERFSAEFPKEGPSILRIQQVVRGDSAAYFCASSTREGTDTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 37 - protein, 135 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRRGYGGSQGNLIFGKGTKLSVKPN

SEQ ID NO: 38 - protein, 275 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRRGYGGSQGNLIFGKGTKLSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 39 - protein, 135 aa, synthetic construct
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLTAGAYNEQFFGPGTRLTVLE

SEQ ID NO: 40 - protein, 313 aa, synthetic construct
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLTAGAYNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 41 - protein, 132 aa, synthetic construct
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAADPRDNYGQNFVFGPGTRLSVLPY

SEQ ID NO: 42 - protein, 272 aa, synthetic construct
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAADPRDNYGQNFVFGPGTRLSVLPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 43 - protein, 134 aa, synthetic construct
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSPLAGGETQYFGPGTRLLVLE

SEQ ID NO: 44 - protein, 312 aa, synthetic construct
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSPLAGGETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 45 - protein, 136 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRTKDYGGSQGNLIFGKGTKLSVKPN

SEQ ID NO: 46 - protein, 276 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRTKDYGGSQGNLIFGKGTKLSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 47 - protein, 137 aa, synthetic construct
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLALPAADSYEQYFGPGTRLTVTE

SEQ ID NO: 48 - protein, 315 aa, synthetic construct
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLALPAADSYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 49 - protein, 136 aa, synthetic construct
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRRFYNTDKLIFGTGTRLQVFPN

SEQ ID NO: 50 - protein, 276 aa, synthetic construct
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCAYRRFYNTDKLIFGTGTRLQVFPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 51 - protein, 132 aa, synthetic construct
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASRSAGYAEAFFGQGTRLTVVE

SEQ ID NO: 52 - protein, 308 aa, synthetic construct
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASRSAGYAEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 53 - protein, 135 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRSGYGGSQGNLIFGKGTKLSVKPN

SEQ ID NO: 54 - protein, 275 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRSGYGGSQGNLIFGKGTKLSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 55 - protein, 136 aa, synthetic construct
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLAGGSLQETQYFGPGTRLLVLE

SEQ ID NO: 56 - protein, 314 aa, synthetic construct
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLAGGSLQETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 57 - protein, 136 aa, synthetic construct; identical to SEQ ID NO: 25

SEQ ID NO: 58 - protein, 276 aa, synthetic construct; identical to SEQ ID NO: 26

SEQ ID NO: 59 - protein, 133 aa, synthetic construct
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASINPRKGGYTFGSGTRLTVVE

SEQ ID NO: 60 - protein, 309 aa, synthetic construct
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASINPRKGGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 61 - protein, 136 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPGNYGGSQGNLIFGKGTKLSVKPN

SEQ ID NO: 62 - protein, 276 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPGNYGGSQGNLIFGKGTKLSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 63 - protein, 135 aa, synthetic construct
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSPPGGLGEKLFFGSGTQLSVLE

SEQ ID NO: 64 - protein, 311 aa, synthetic construct
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSPPGGLGEKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 65 - protein, 136 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPVNYGGSQGNLIFGKGTKLSVKPN

SEQ ID NO: 66 - protein, 276 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPVNYGGSQGNLIFGKGTKLSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 67 - protein, 133 aa, synthetic construct
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASRSPRLETQYFGPGTRLLVLE

SEQ ID NO: 68 - protein, 311 aa, synthetic construct
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASRSPRLETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 69 - protein, 136 aa, synthetic construct; identical to SEQ ID NO: 45

SEQ ID NO: 70 - protein, 276 aa, synthetic construct; identical to SEQ ID NO: 46

SEQ ID NO: 71 - protein, 135 aa, synthetic construct
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSFPGLVSYEQYFGPGTRLTVTE

SEQ ID NO: 72 - protein, 313 aa, synthetic construct
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSFPGLVSYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 73 - protein, 135 aa, synthetic construct; identical to SEQ ID NO: 53

SEQ ID NO: 74 - protein, 275 aa, synthetic construct; identical to SEQ ID NO: 54

SEQ ID NO: 75 - protein, 133 aa, synthetic construct
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLAGQETQYFGPGTRLLVLE

SEQ ID NO: 76 - protein, 311 aa, synthetic construct
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLAGQETQYFGPGTRLLVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 77 - protein, 135 aa, synthetic construct; identical to SEQ ID NO: 29

SEQ ID NO: 78 - protein, 275 aa, synthetic construct; identical to SEQ ID NO: 30

SEQ ID NO: 79 - protein, 135 aa, synthetic construct; identical to SEQ ID NO: 31

SEQ ID NO: 80 - protein, 311 aa, synthetic construct; identical to SEQ ID NO: 32

SEQ ID NO: 81 - protein, 136 aa, synthetic construct; identical to SEQ ID NO: 25

SEQ ID NO: 82 - protein, 276 aa, synthetic construct; identical to SEQ ID NO: 26

SEQ ID NO: 83 - protein, 134 aa, synthetic construct
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLAERPGTQYFGPGTRLTVLE

SEQ ID NO: 84 - protein, 312 aa, synthetic construct
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLAERPGTQYFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 85 - protein, 136 aa, synthetic construct; identical to SEQ ID NO: 25

SEQ ID NO: 86 - protein, 276 aa, synthetic construct; identical to SEQ ID NO: 26

SEQ ID NO: 87 - protein, 135 aa, synthetic construct; identical to SEQ ID NO: 31

SEQ ID NO: 88 - protein, 311 aa, synthetic construct; identical to SEQ ID NO: 32

(19) United States
(12) Patent Application Publication
(10) Pub. No.: US 2025/0242021 A1
(43) Pub. Date: Jul. 31, 2025

(54) ALLOGENEIC T CELLS FOR TREATMENT OF HEMATOLOGICAL MALIGNANCIES

(71) Applicants: BLUESPHERE BIO, INC., Pittsburgh, PA (US); UNIVERSITY OF PITTSBURGH- OF THE COMMONWEALTH SYSTEM OF HIGHER EDUCATION, Pittsburgh, PA (US)

(72) Inventors: Tim MAYALL, Encinitas, CA (US); Paola SETTE, Pittsburgh, PA (US); Mark SHLOMCHIK, Pittsburgh, PA (US); Warren David SHLOMCHIK, Pittsburgh, PA (US)

(21) Appl. No.: 18/857,000

(22) PCT Filed: Apr. 19, 2023
(86) PCT No.: PCT/US2023/065967

§ 371 Date: Oct. 15, 2024

Related U.S. Application Data
(60) Provisional application No. 63/478,588, filed on Jan. 5, 2023.
(60) Provisional application No. 63/383,805, filed on Nov. 15, 2022.
(60) Provisional application No. 63/392,738, filed on Jul. 27, 2022.
(60) Provisional application No. 63/333,036, filed on Apr. 20, 2022.
(60) Provisional application No. 63/333,035, filed on Apr. 20, 2022.
(60) Provisional application No. 63/333,031, filed on Apr. 20, 2022.

(51) Int. Cl. A61K 40/11; A61K 40/32; A61K 40/41; A61K 40/50; A61P 35/02; C07K 14/725; C12N 5/0783; C12N 15/86

(52) U.S. Cl. CPC A61K 40/11; A61K 40/32; A61K 40/418; A61K 40/50; A61P 35/02; C07K 14/7051; C12N 5/0636; C12N 15/86; C12N 2510/00; C12N 2740/15043

(57) ABSTRACT

Described are methods of manufacturing engineered T cells and methods of using the engineered T cells to treat hematological malignancies. The engineered T cells can be administered in combination with allogeneic stem cell transplant and reduce the need for prophylactic immunosuppression.

### CROSS-REFERENCE TO RELATED APPLICATIONS

This application claims the benefit of U.S. Provisional Application Ser. No. 63/333,031, filed Apr. 20, 2022, U.S. Provisional Application Ser. No. 63/333,035, filed Apr. 20, 2022, U.S. Provisional Application Ser. No. 63/333,036, filed Apr. 20, 2022, U.S. Provisional Application Ser. No. 63/392,738, filed Jul. 27, 2022, U.S. Provisional Application Ser. No. 63/383,805, filed Nov. 15, 2022, and U.S. Provisional Application Ser. No. 63/478,588, filed Jan. 5, 2023, each of which is incorporated herein by reference.

### SEQUENCE LISTING

The Sequence Listing written in file BSB-0003WO01_SeqListing_ST26.xml is 110 kilobytes in size, was created Apr. 18, 2023, and is hereby incorporated by reference.

### BACKGROUND

Chemotherapy or chemotherapy combined with radiation therapy followed by infusion of hematopoietic stem cells harvested from a related or unrelated allogeneic donor, is a widely used therapeutic strategy to cure or extend survival in patients with hematological malignancies such as acute myeloid leukemia (AML), acute lymphoblastic leukemia (ALL) and high risk myelodysplastic syndromes (MDS). Much of the efficacy of such allogeneic hematopoietic stem cell transplants (alloSCTs) is due to the activity of alloreactive T cells in the donor graft, which can kill recipient leukemia cells, thereby reducing the risk of leukemia relapse. This immune effect is called the graft-versus-leukemia (GVL) response. More than 9000 alloSCTs were performed in the United States (US) in 2019, mostly as a potentially curative treatment for patients with AML, MDS and ALL. Despite alloSCT being shown to be superior to not receiving a transplant due to a reduction in malignant disease recurrence, relapsed malignant disease remains the largest single cause of transplant failure, occurring in 20-40% of standard risk and in 40-80% of high-risk patients, accounting for more than half of deaths after alloSCT. There is therefore an urgent need to prevent and treat post-transplant relapse through new strategies that can augment the GVL effect without substantially increasing the risk for severe graft-versus-host disease (GVHD).

### SUMMARY

Described are methods of treating a recipient subject having cancer, the methods comprising: (a) isolating CD8⁺ T cells from an apheresis product collected from a donor subject HLA-matched to a recipient subject, (b) activating the isolated CD8⁺ T cells; (c) genetically modifying the activated T cells to knock out the endogenous TCR and insert a nucleic acid sequence encoding a heterologous TCR or CAR, thereby generating engineered T cells, wherein the engineered T cells express the heterologous TCR or CAR but do not express the endogenous TCR; (d) expanding the engineered T cells; and (e) administering or having administered the engineered T cells to the recipient subject. In some embodiments, the apheresis product comprises a leukapheresis product. In some embodiments, peripheral blood mononuclear cells (PBMCs) are isolated from the apheresis product. Inserting the nucleic acid sequence encoding a heterologous TCR or CAR results in expression of the heterologous TCR or CAR in the T cell. In some embodiments, the engineered T cells are administered to the recipient subject in combination with alloSCT. In some embodiments, the alloSCT comprises a CD34-selected alloSCT graft. The alloSCT graft, or the CD34-selected alloSCT graft, can be derived from the same donor as the apheresis product of step (a). In some embodiments, the recipient subject has a hematopoietic malignancy (also termed hematological malignancy). The hematopoietic cancer can be, but is not limited to, AML, ALL, or MDS.

Described are methods of treating a recipient subject having a hematological malignancy comprising: (a) administering a CD34-selected allogeneic stem cell transplant (alloSCT) graft to the recipient subject, wherein the alloSCT graft is obtained from a graft donor subject that is HLA-matched to the recipient subject; and (b) administering engineered T cells to the recipient subject, wherein the engineered T cells express a heterologous TCR or chimeric antigen receptor (CAR) that recognizes a hematopoietic restricted minor histocompatibility antigen (miHA) expressed by the recipient subject; wherein the engineered T cells are administered to the recipient subject within 72 hours of administering the CD34-selected alloSCT graft to the recipient subject, and wherein no immunosuppressive agent that targets Graft versus Host Disease (GVHD) is prophylactically administered to the recipient subject to suppress the immune activity of T cells. In some embodiments, the engineered T cells do not express the endogenous TRAC or TRBC genes or both. The recipient subject can be administered a conditioning regimen prior to administration of the CD34-selected alloSCT graft. In some embodiments, the recipient subject is in condition for hematopoietic stem cell transplant. In some embodiments, the engineered T cells are administered to the recipient subject within 48 hours of administering the CD34-selected alloSCT graft to the recipient subject. In some embodiments, the engineered T cells are administered to the recipient subject within 24 hours of administering the CD34-selected alloSCT graft to the recipient subject. In some embodiments, the engineered T cells are administered to the recipient subject within 18 hours of administering the CD34-selected alloSCT graft to the recipient subject. In some embodiments, the engineered T cells are administered to the recipient subject within 12 hours of administering the CD34-selected alloSCT graft to the recipient subject. In some embodiments, the engineered T cells are administered to the recipient subject within 6 hours of administering the CD34-selected alloSCT graft to the recipient subject.

The donor subject can be a related donor having an HLA 10/10 match to the recipient subject, an unrelated donor having an HLA 10/10 match to the recipient subject, a related donor having an HLA 11/12 match to the recipient subject, a related donor having an HLA 12/12 match to the recipient subject, an unrelated donor having an HLA 11/12 match to the recipient subject, or an unrelated donor having an HLA 12/12 match to the recipient subject. A donor subject having an HAL 11/12 match to the recipient subject shares a single mismatch with the recipient subject at the HLA-DQ or the HLA-DP locus in the graft-versus-host direction (i.e., wherein the donor subject is homozygous for the mismatched allele and the recipient subject is heterozygous for the mismatch allele).

The engineered T cells can be derived from a T cell donor subject that is HLA-matched to the recipient subject. The alloSCT graft donor subject and the T cell donor subject can be the same donor subject. In some embodiments, the donor subject can undergo a first apheresis procedure to collect an apheresis product for manufacturing the engineered T cells and a second apheresis procedure to collect an apheresis product for the graft that provides donor hematopoiesis in the recipient (e.g., alloSCT graft). The first apheresis procedure may be performed without first administering a reagent known to mobilize hematopoietic stem cells (mobilizing agent) to the donor subject. The donor subject may be administered a mobilizing agent prior to the second apheresis. The apheresis product collected for the alloSCT graft can be processed to select CD34⁺ cells thereby generating the CD34-selected alloSCT graft. The apheresis product can be a leukapheresis product (e.g., PBMCs).

Exemplary hematopoietic restricted miHAs include, but are not limited to, a HA-1 epitope or a HA-2 epitope. A HA-1 epitope can have the amino acid sequence of SEQ ID NO: 3. A HA-2 epitope can have the amino acid sequence of SEQ ID NO: 5.

An exemplary TCR that recognizes an HA-1 epitope includes, but is not limited to, a TCR comprising an alpha variable region having the amino acid SEQ ID NO: 14 and a beta variable region having the amino acid sequence of SEQ ID NO: 8. Such an anti-HA-1 TCR can comprise an alpha chain having the amino acid sequence of SEQ ID NO: 18 and a beta chain having the amino acid sequence of SEQ ID NO: 12. The alpha chain and the beta chain can be expressed from a single promoter. In some embodiments, the alpha and beta TCR chains are encoded on a single transcript and separated by a 2A element (e.g., a p2A or T2A element). In some embodiments, an engineered T cell expressing an anti-HA-1 antigen TCR comprises an expressible nucleic acid sequence encoding the amino acid sequence of SEQ ID NO: 20.

An exemplary TCR that recognizes an HA-2 epitope includes, but is not limited to, a TCR comprising:
(a) an alpha variable region having the amino acid SEQ ID NO: 25 and a beta variable region having the amino acid sequence of SEQ ID NO: 27;
(b) an alpha variable region having the amino acid SEQ ID NO: 29 and a beta variable region having the amino acid sequence of SEQ ID NO: 31;
(c) an alpha variable region having the amino acid SEQ ID NO: 33 and a beta variable region having the amino acid sequence of SEQ ID NO: 35;
(d) an alpha variable region having the amino acid SEQ ID NO: 37 and a beta variable region having the amino acid sequence of SEQ ID NO: 39;
(e) an alpha variable region having the amino acid SEQ ID NO: 41 and a beta variable region having the amino acid sequence of SEQ ID NO: 43;
(f) an alpha variable region having the amino acid SEQ ID NO: 45 and a beta variable region having the amino acid sequence of SEQ ID NO: 47;
(g) an alpha variable region having the amino acid SEQ ID NO: 49 and a beta variable region having the amino acid sequence of SEQ ID NO: 51;
(h) an alpha variable region having the amino acid SEQ ID NO: 53 and a beta variable region having the amino acid sequence of SEQ ID NO: 55;
(i) an alpha variable region having the amino acid SEQ ID NO: 57 and a beta variable region having the amino acid sequence of SEQ ID NO: 59;
(j) an alpha variable region having the amino acid SEQ ID NO: 61 and a beta variable region having the amino acid sequence of SEQ ID NO: 63;
(k) an alpha variable region having the amino acid SEQ ID NO: 65 and a beta variable region having the amino acid sequence of SEQ ID NO: 67;
(l) an alpha variable region having the amino acid SEQ ID NO: 69 and a beta variable region having the amino acid sequence of SEQ ID NO: 71;
(m) an alpha variable region having the amino acid SEQ ID NO: 73 and a beta variable region having the amino acid sequence of SEQ ID NO: 75;
(n) an alpha variable region having the amino acid SEQ ID NO: 77 and a beta variable region having the amino acid sequence of SEQ ID NO: 79;
(o) an alpha variable region having the amino acid SEQ ID NO: 81 and a beta variable region having the amino acid sequence of SEQ ID NO: 83; or
(p) an alpha variable region having the amino acid SEQ ID NO: 85 and a beta variable region having the amino acid sequence of SEQ ID NO: 87.

In some embodiments, the alpha and beta regions or an anti-HA-2 TCR are expressed from a single transcript in an engineered T cell. In some embodiments, the alpha and beta TCR chains are encoded on a single transcript and separated by a 2A element (e.g., a p2A or T2A element).

An exemplary TCR that recognizes an HA-2 epitope includes, but is not limited to, a TCR comprising:
(a) an alpha chain having the amino acid SEQ ID NO: 26 and a beta chain having the amino acid sequence of SEQ ID NO: 28;
(b) an alpha chain having the amino acid SEQ ID NO: 30 and a beta chain having the amino acid sequence of SEQ ID NO: 32;
(c) an alpha chain having the amino acid SEQ ID NO: 34 and a beta chain having the amino acid sequence of SEQ ID NO: 36;
(d) an alpha chain having the amino acid SEQ ID NO: 38 and a beta chain having the amino acid sequence of SEQ ID NO: 40;
(e) an alpha chain having the amino acid SEQ ID NO: 42 and a beta chain having the amino acid sequence of SEQ ID NO: 44;
(f) an alpha chain having the amino acid SEQ ID NO: 46 and a beta chain having the amino acid sequence of SEQ ID NO: 48;
(g) an alpha chain having the amino acid SEQ ID NO: 50 and a beta chain having the amino acid sequence of SEQ ID NO: 52;
(h) an alpha chain having the amino acid SEQ ID NO: 54 and a beta chain having the amino acid sequence of SEQ ID NO: 56;
(i) an alpha chain having the amino acid SEQ ID NO: 58 and a beta chain having the amino acid sequence of SEQ ID NO: 60;
(j) an alpha chain having the amino acid SEQ ID NO: 62 and a beta chain having the amino acid sequence of SEQ ID NO: 64;
(k) an alpha chain having the amino acid SEQ ID NO: 66 and a beta chain having the amino acid sequence of SEQ ID NO: 68;
(l) an alpha chain having the amino acid SEQ ID NO: 70 and a beta chain having the amino acid sequence of SEQ ID NO: 72;
(m) an alpha chain having the amino acid SEQ ID NO: 74 and a beta chain having the amino acid sequence of SEQ ID NO: 76;
(n) an alpha chain having the amino acid SEQ ID NO: 78 and a beta chain having the amino acid sequence of SEQ ID NO: 80;
(o) an alpha chain having the amino acid SEQ ID NO: 82 and a beta chain having the amino acid sequence of SEQ ID NO: 84; or
(p) an alpha chain having the amino acid SEQ ID NO: 86 and a beta chain having the amino acid sequence of SEQ ID NO: 88.

In some embodiments, the nucleic acid sequences encoding the alpha and beta chains of an anti-HA-2 TCR are provided on a single vector or expression cassette and expressed from a single promoter. In some embodiments, the alpha and beta TCR chains are encoded on a single transcript and separated by a 2A element (e.g., a p2A or T2A element).

In some embodiments, the engineered T cells further expresses an RQR8 peptide. The RQR8 peptide can be expressed from the same promoter as the heterologous TCR. In some embodiments, the alpha and beta TCR chains and the RQR8 peptide are encoded on a single transcript and separated by a 2A elements (e.g., a p2A or T2A element). In some embodiments, an engineered T cell expressing an anti-HA-1 antigen TCR and a RQR8 peptide comprises an expressible nucleic acid sequence encoding the amino acid sequence of SEQ ID NO: 22. In some embodiments, an engineered T cell expressing an anti-HA-1 antigen TCR and a RQR8 peptide comprises an expressible nucleic acid sequence comprising SEQ ID NO: 21.

A nucleic acid sequence encoding the heterologous TCR can be operably linked to a promoter to express to heterologous TCR in the engineered T cell. The promoter can be any promoter that is active in T cells. The promoter can be, but is not limited to, a MNDU3 promoter. In some embodiments, an engineered T cell expressing an anti-HA-1 TCR and a RQR8 peptide from a MNDU3 promoter comprises a nucleic acid sequence comprising SEQ ID NO: 23.

In some embodiments, the described methods further comprise: (i) performing a first apheresis procedure on a T cell donor subject to collect an apheresis product; (ii) isolating CD8⁺ T cells from the apheresis product; (iii) activating the CD8⁺ T cells; (iv) genetically modifying the CD8⁺ cells to knock out endogenous TRAC and TRBC genes; (v) genetically modifying the CD8⁺ T cells to express a heterologous TCR or CAR thereby generating engineered T cells; and (vi) expanding the engineered T cells prior to administering the engineered T cells to the recipient subject.

Described are methods of treating a recipient subject suffering from a hematological malignancy comprising: (a) genetically modifying CD8⁺ T cells from a donor apheresis product to knock out the endogenous TRAC and TRBC genes and express a heterologous TCR that recognizes a hematopoietic-restricted miHA antigen expressed by the recipient subject thereby generating engineered T cells, wherein the donor apheresis product is obtained from a donor subject that is HLA-matched to the recipient subject but does not express the hematopoietic-restricted miHA antigen; and (b) administering a CD34-selected alloSCT graft and the engineered T cells to the recipient subject, wherein the CD34-selected alloSCT graft comprises CD34⁺ cells obtained from the donor subject and wherein the engineered T cells are administered to the recipient subject within 72 hours of administering the CD34-selected alloSCT graft to the recipient subject; wherein no immunosuppressive agent that targets GVHD is prophylactically administered to the recipient subject to suppress the immune activity of T cells. In some embodiments, the engineered T cells are administered to the recipient subject within 12 hours of administering the CD34-selected alloSCT graft to the recipient subject. In some embodiments, the engineered T cells are administered to the recipient subject within 12 hours of administering the CD34-selected alloSCT graft to the recipient subject. In some embodiments, the engineered T cells are administered to the recipient subject within 6 hours of administering the CD34-selected alloSCT graft to the recipient subject.

The described methods can be used to treat a recipient subject having a hematological malignancy, such as, leukemia, acute lymphoblastic leukemia (ALL), acute myeloid leukemia (AML), myelodysplastic syndrome (MDS), chronic lymphocytic leukemia, chronic myelogenous leukemia, chronic myeloid leukemia in blast crisis, chronic myeloid leukemia in accelerated phase, multiple myeloma, or non-Hodgkin's lymphoma.

In some embodiments, the recipient subject has no measurable residual disease (MRD).

In some embodiments, the recipient subject has MRD or morphologically identifiable disease. In some embodiments, the recipient subject has ≥5% myeloblasts in their bone marrow and/or detectable myeloblasts in their peripheral blood. In some embodiments, the recipient subject has ≥10% myeloblasts in their bone marrow and/or detectable myeloblasts in their peripheral blood. In some embodiments, the recipient subject has 15% myeloblasts in their bone marrow and/or detectable myeloblasts in their peripheral blood. In some embodiments, the recipient subject has ≥20% myeloblasts in their bone marrow and/or detectable myeloblasts in their peripheral blood. In some embodiments, the recipient subject has ≤25% myeloblasts in their bone marrow. In some embodiments, the recipient subject has about 5% to about 25% myeloblasts in their bone marrow In some embodiments, the recipient subject has AML, ALL, or MRD, and up to 25% myeloblasts in bone marrow. The percentage of myeloblasts can be determined by any known method.

In some embodiments, the recipient subject is refractory to at least one prior therapy. The at least one prior therapy can be, but is not limited to, induction therapy and/or consolidation therapy. Accordingly, in embodiments, the methods described herein can be used to treat a refractory hematological malignancy.

In some embodiments, the recipient subject has one or more risk factors or indicators of poor outcome typically associated with standard alloSCT therapy. The risk factors or indicators of poor outcome include, but are not limited to, a TP53 mutation, a complex karyotype, a typical complex karyotype, an atypical complex karyotype, a monosomal karyotype, a 17p chromosomal abnormality, and a Ph+ chromosomal abnormality. The risk factors or indicators of poor prognosis can be determined using methods available in the art. The risk factors or indicators of poor prognosis can be determined prior to or after diagnosis of a hematopoietic malignancy. The risk factors or indicators of poor prognosis can be determined prior to or following administration of a treatment for the hematopoietic malignancy. The risk factors or indicators of poor prognosis can be determined prior to administering alloSCT to the recipient subject. In some embodiments, a recipient subject suffering from a hematopoietic malignancy is tested for the presence of one or more of the risk factors or indicators of poor prognosis prior to administering alloSCT or alloSCT in combination with engineered T cells. In some embodiments, the presence or absence of one or more of the risk factors or the indicators of poor prognosis in a recipient subject suffering from a hematopoietic malignancy is determined, wherein the presence of one or more of the risk factors or the indicators of poor prognosis indicates the recipient subject is a candidate for treatment with alloSCT in combination with engineered T cells as described.

In some embodiments, the recipient subject has AML and at least one risk factor, and/or detectable RUNX1-RUNX1T1 transcripts, CBFB-MYH11 transcripts, NPM1 mutant transcripts, or FLT3-ITD transcripts in blood or bone marrow. The risk factor can be, but is not limited to, a TP53 mutation, a complex karyotype, a monosomal karyotype, a 17p chromosomal abnormality, a Ph+ chromosomal abnormality, and a MECOM (EVI1) rearrangement.

In some embodiments, the recipient subject has ALL and at least one risk factor. The risk factor can be, but is not limited to, a persistent disease-defining cytogenetic abnormality, a Ph+ chromosomal abnormality, a CRLF2 mutation, an Ikaros deletion, monosomy 7, or a complex karyotype. In some embodiments, the recipient subject has ALL and persistent disease based on sensitive molecular techniques.

In some embodiments, the recipient subject has MDS and at least one risk factor. The risk fact can be, but is not limited to, a complex karyotype, a monosomal karyotype, a TP53 mutation, an RAS-pathway mutation, a JAK2 mutation, a RUNX1 mutation, or a ASXL1 mutation.

Described are methods of manufacturing engineered T cells for administration to a recipient subject comprising: (a) identifying a donor subject that is HLA-matched to the recipient subject or having a donor subject that is HLA-matched to the recipient subject; (b) collecting an apheresis product from the donor subject or having an apheresis product collected from the donor subject; (c) selecting CD8⁺ T cells from the apheresis product to form an enriched CD8⁺ T cell population; (d) activating the CD8⁺ T cells in the enriched CD8⁺ T cell population; (e) genetically modifying the CD8⁺ T cells to express a nucleic acid encoding a heterologous TCR or chimeric antigen receptor (CAR) thereby the generating the engineered T cells, wherein the engineered T cells express the heterologous TCR or CAR; and (f) expanding the engineered T cells. The apheresis product can be a leukapheresis product. In some embodiments, PBMCs are isolated from the apheresis product prior to CD8+ selection. In some embodiments, the apheresis is performed without prior administration of a mobilizing agent to the donor subject.

In some embodiments, isolating T cells (e.g., CD8⁺ T cells), knocking out the endogenous TCR, and inserting the nucleic acid encoding the heterologous TCR or CAR are performed within 36-72 hours of the apheresis product being collected. In some embodiments, isolating T cells, knocking out the endogenous TCR, and inserting the nucleic acid encoding the heterologous TCR or CAR are performing within 18-36 hours of the contacting activating the CD8⁺ T cells. In some embodiments, the nucleic acid encoding the heterologous TCR or CAR is inserted into the CD8⁺ T cells within about 24 hours of knocking out the endogenous TCR. In some embodiments, steps (c), (d), and (e) are performed 24-48 hours after step (b). In some embodiments, expanding the engineered T cells comprises incubating the engineered T cells in conditions suitable for growth for about 7 to about 14 days.

The donor subject can be a related donor having an HLA 10/10 match to the recipient subject, an unrelated donor having an HLA 10/10 match to the recipient subject, a related donor having an HLA 11/12 match to the recipient subject, a related donor having an HLA 12/12 match to the recipient subject, a unrelated donor having an HLA 11/12 match to the recipient subject, or a unrelated donor having an HLA 12/12 match to the recipient subject. A donor subject having an HLA 11/12 match to the recipient subject shares a single mismatch with the recipient subject at the HLA-DQ or the HLA-DP locus in the graft-versus-host direction (i.e., wherein the donor subject is homozygous for the mismatch allele and the recipient subject is heterozygous for the mismatch allele).

In some embodiments, activating the CD8⁺ T cells comprises contacting the CD8⁺ T cells with: a soluble anti-CD3 antibody in the absence of a CD28 agonist; a CD3 agonist and a CD28 agonist; or immobilized CD3 and CD28 agonists. The CD8⁺ T cells can be incubated with the activating agents for about 12 to about 24 hours.

In some embodiments, the methods further comprises knocking out the endogenous T cell receptor (TCR) in the activated CD8⁺ T cells after step (d) and before step (e). Knocking out the endogenous T cell receptor in the isolated CD8⁺ T cells can be done using a CRISPR system. Both the TCR α chain and the TCR β chain can be knocked out using CRISPR systems. The CRISPR system can comprise an RNA-guided DNA endonuclease enzyme and a guide RNA. The RNA-guided DNA endonuclease enzyme can be, but is not limited to, Cas9 enzyme. The guide RNA can be, but is not limited to, SEQ ID NO: 1 and SEQ ID NO: 2. In some embodiments, knocking out the endogenous T cell receptor can comprise transfecting the CD8⁺ T cells with an Cas9/guide RNA pre-formulated ribonucleoprotein complex targeting the TRAC and TRBC genes, wherein the pre-formulated ribonucleoprotein complex a guide RNA comprising SEQ ID NO: 1 and a guide RNA comprises SEQ ID NO: 2.

Inserting a nucleic acid sequence encoding the heterologous TCR (or an antigen binding fragment thereof) or CAR can be performed using methods available in the art for introducing a heterologous nucleic acid sequence into a T cell. In some embodiments, genetically modifying the CD8⁺ T cells to express the heterologous TCR or CAR comprises introducing a vector encoding the heterologous TCR or CAR into the CD8⁺ T cells. In some embodiments, inserting a nucleic acid sequence encoding the heterologous TCR or CAR comprises transducing the T cells with a viral vector containing a heterologous nucleic acid sequence encoding the heterologous TCR or CAR. The viral vector can be, but is not limited to, a lentiviral vector.

In some embodiments, the genetically modified the CD8⁺ T cells contain 5 or fewer copies of a nucleic acid encoding the heterologous TCR or CAR. In some embodiments, the CD8⁺ T cells are transduced with a lentiviral vector encoding the heterologous TCR or CAR at a multiplicity of infection that results in the T cells containing 5 or fewer copies of a nucleic acid encoding the heterologous TCR or CAR.

The heterologous TCR can be, but is not limited to, an αβ TCR or an antigen binding fragment thereof. The heterologous TCR can be, but is not limited to, an anti-miHA antigen TCR, an anti-miHA HA-1 TCR, an anti-miHA HA-2, an anti-viral antigen TCR, or an anti-tumor neoantigen TCR. The CAR can be, but is not limited to, first, second, third or fourth generation CAR. The CAR can bind to, for example, a tumor neoantigen, or viral antigen, or a miHA antigen. In some embodiments, the heterologous TCR is derived from a tumor infiltrating T cell from a subject.

Engineered T cells manufactured using the described methods can be administered to a recipient subject in need of or who would benefit from therapeutic T cell therapy. In some embodiments, the engineered T cells are used in combination with alloSCT or CD34-selected alloSCT. Engineered T cells manufactured used the described methods can be used to increase graft versus leukemia effect in a recipient subject treated with allogeneic hematopoietic stem cell transplantation (alloSCT) and/or to reduce the risk of graft versus host disease in a recipient subject treated with alloSCT.

In some embodiments, the heterologous TCR or CAR comprises a TCR or CAR having affinity for a hematopoietic restricted miHA antigen expressed by the recipient subject. Exemplary hematopoietic restricted miHA antigens include, but are not limited to, a HA-1 epitope or an HA-2 epitope. An HA-1 epitope can have the amino acid sequence of SEQ ID NO: 3. An HA-1 epitope can have the amino acid sequence of SEQ ID NO: 5.

An exemplary TCR that recognizes an HA-1 epitope includes, but is not limited to, a TCR comprising an alpha variable region having the amino acid SEQ ID NO: 14 and a beta variable region having the amino acid sequence of SEQ ID NO: 8. Such an anti-HA-1 TCR can comprise an alpha chain having the amino acid sequence of SEQ ID NO: 18 and a beta chain having the amino acid sequence of SEQ ID NO: 12. The alpha chain and the beta chain can be expressed from a single promoter. In some embodiments, the alpha and beta TCR chains are encoded on a single transcript and separated by a 2A element (e.g., a p2A or T2A element). In some embodiments, an engineered T cell expressing an anti-HA-1 antigen TCR comprises an expressible nucleic acid sequence encoding the amino acid sequence of SEQ ID NO: 20.

In some embodiments, an exemplary engineered T cell expressing an anti-HA-2 antigen TCR comprises an expressible nucleic acid sequence encoding:
(a) an alpha variable region having the amino acid SEQ ID NO: 25 and a beta variable region having the amino acid sequence of SEQ ID NO: 27;
(b) an alpha variable region having the amino acid SEQ ID NO: 29 and a beta variable region having the amino acid sequence of SEQ ID NO: 31;
(c) an alpha variable region having the amino acid SEQ ID NO: 33 and a beta variable region having the amino acid sequence of SEQ ID NO: 35;
(d) an alpha variable region having the amino acid SEQ ID NO: 37 and a beta variable region having the amino acid sequence of SEQ ID NO: 39;
(e) an alpha variable region having the amino acid SEQ ID NO: 41 and a beta variable region having the amino acid sequence of SEQ ID NO: 43;
(f) an alpha variable region having the amino acid SEQ ID NO: 45 and a beta variable region having the amino acid sequence of SEQ ID NO: 47;
(g) an alpha variable region having the amino acid SEQ ID NO: 49 and a beta variable region having the amino acid sequence of SEQ ID NO: 51;
(h) an alpha variable region having the amino acid SEQ ID NO: 53 and a beta variable region having the amino acid sequence of SEQ ID NO: 55;
(i) an alpha variable region having the amino acid SEQ ID NO: 57 and a beta variable region having the amino acid sequence of SEQ ID NO: 59;
(j) an alpha variable region having the amino acid SEQ ID NO: 61 and a beta variable region having the amino acid sequence of SEQ ID NO: 63;
(k) an alpha variable region having the amino acid SEQ ID NO: 65 and a beta variable region having the amino acid sequence of SEQ ID NO: 67;
(l) an alpha variable region having the amino acid SEQ ID NO: 69 and a beta variable region having the amino acid sequence of SEQ ID NO: 71;
(m) an alpha variable region having the amino acid SEQ ID NO: 73 and a beta variable region having the amino acid sequence of SEQ ID NO: 75;
(n) an alpha variable region having the amino acid SEQ ID NO: 77 and a beta variable region having the amino acid sequence of SEQ ID NO: 79;
(o) an alpha variable region having the amino acid SEQ ID NO: 81 and a beta variable region having the amino acid sequence of SEQ ID NO: 83; or
(p) an alpha variable region having the amino acid SEQ ID NO: 85 and a beta variable region having the amino acid sequence of SEQ ID NO: 87.

In some embodiments, the alpha and beta regions of the anti-HA-2 TCR are expressed from a single transcript in the engineered T cell. In some embodiments, the alpha and beta TCR chains are encoded on a single transcript and separated by a 2A element (e.g., a p2A or T2A element).

In some embodiments, the engineered T cells further expresses an RQR8 peptide. The RQR8 peptide can be express from the sample promoter as the heterologous TCR. In some embodiments, the alpha and beta TCR chains and the RQR8 peptide are encoded on a single transcript and separated by a 2A elements (e.g., a p2A or T2A element). In some embodiments, an engineered T cell expressing an anti-HA-1 antigen TCR and an RQR8 peptide comprises an expressible nucleic acid sequence encoding the amino acid sequence of SEQ ID NO: 22. In some embodiments, an engineered T cell expressing an anti-HA-1 antigen TCR and an RQR8 peptide comprises an expressible nucleic acid sequence comprising SEQ ID NO: 21.

A nucleic acid sequence encoding the heterologous TCR can be operably linked to a promoter to express to heterologous TCR in the engineered T cell. The promoter can be any promoter that is active in T cells. The promoter can be, but is not limited to, an MNDU3 promoter. In some embodiments, an engineered T cell expressing an anti-HA-1 antigen TCR and an RQR8 peptide from an MNDU3 promoter comprises a nucleic acid sequence comprising SEQ ID NO: 23.

In some embodiments, about 10% to about 90%, or at least 50%, of engineered T cells manufactured using the described methods have a stem-cell like phenotype. In some embodiments, the engineered T cells kill cells expressing an antigen recognized by the heterologous TCR or CAR at an Effector:Target ratio less than 1:1, or less than 0.5:1. In some embodiments, the engineered T cells maintain at least 50% of their cytolytic potency over a period of a least 20 days.

In some embodiments, the engineered T cells are generated, expanded, and cryopreserved within about 9 to about 15 days of collecting the apheresis product from a donor subject. If cryopreserved, the engineered T cells are thawed prior to administration to the recipient subject. In some embodiments, at least 10%, at least 20%, at least 30%, at least 40%, or at least 50% of the thawed engineered T cells have a stem-cell like phenotype. In some embodiments, the thawed engineered T cells kill cells expressing an antigen recognized by the heterologous TCR or CAR at an Effector:Target ratio of less than 1:1. In some embodiments, the thawed engineered T cells kill cells expressing an antigen recognized by the heterologous TCR or CAR at an Effector:Target ratio less than 0.5:1. In some embodiments, the thawed engineered T cells maintain at least 50% of their cytolytic potency for at least 20 days.

Engineered T cells manufactured using the described methods can be used in T cell therapies. The T cell therapies can be used to treat a recipient subject having a hematological malignancy comprising. In some embodiments, the engineered T cells are administered to a recipient subject in combination with alloSCT. The alloSCT can be CD34-selected alloSCT. In some embodiments, the engineered T cells are administered to a recipient subject in combination CD34-selected alloSCT wherein the engineered T cells are administered to the recipient subject on the same day as the CD34-selected alloSCT, and wherein no immunosuppressive agent that targets GVHD is prophylactically administered to the recipient subject to suppress the immune activity of T cells. The hematological malignancy can be, but is not limited to: acute myeloid leukemia, myelodysplastic syndrome, or acute lymphoblastic leukemia.

Also described are nucleic acids encoding anti-HA-1 TCRs. In some embodiments, the nucleic acid comprises a sequence encoding SEQ ID NO: 20 or SEQ ID NO: 22. In some embodiments, the nucleic acid comprises a sequence having at least 75% identity to SEQ ID NO: 19, SEQ ID NO: 21, SEQ ID NO: 23, or SEQ ID NO: 24 and encoding the amino acid sequence of SEQ ID NO: 20 or SEQ ID NO: 22; or a sequence comprising SEQ ID NO: 19, SEQ ID NO: 21, SEQ ID NO: 23, or SEQ ID NO: 24. In some embodiments, the nucleic acid comprises the sequence of SEQ ID NO: 7 or SEQ ID NO: 11. In some embodiments, the nucleic acid comprises the sequence of SEQ ID NO: 13 or SEQ ID NO: 17.

Also described are nucleic acids encoding anti-HA-2 TCRs. In some embodiments, an exemplary nucleic acid encoding an anti-HA-2 TCRs comprises a sequence encoding:
(a) an alpha variable region having the amino acid SEQ ID NO: 25 and a beta variable region having the amino acid sequence of SEQ ID NO: 27;
(b) an alpha variable region having the amino acid SEQ ID NO: 29 and a beta variable region having the amino acid sequence of SEQ ID NO: 31;
(c) an alpha variable region having the amino acid SEQ ID NO: 33 and a beta variable region having the amino acid sequence of SEQ ID NO: 35;
(d) an alpha variable region having the amino acid SEQ ID NO: 37 and a beta variable region having the amino acid sequence of SEQ ID NO: 39;
(e) an alpha variable region having the amino acid SEQ ID NO: 41 and a beta variable region having the amino acid sequence of SEQ ID NO: 43;
(f) an alpha variable region having the amino acid SEQ ID NO: 45 and a beta variable region having the amino acid sequence of SEQ ID NO: 47;
(g) an alpha variable region having the amino acid SEQ ID NO: 49 and a beta variable region having the amino acid sequence of SEQ ID NO: 51;
(h) an alpha variable region having the amino acid SEQ ID NO: 53 and a beta variable region having the amino acid sequence of SEQ ID NO: 55;
(i) an alpha variable region having the amino acid SEQ ID NO: 57 and a beta variable region having the amino acid sequence of SEQ ID NO: 59;
(j) an alpha variable region having the amino acid SEQ ID NO: 61 and a beta variable region having the amino acid sequence of SEQ ID NO: 63;
(k) an alpha variable region having the amino acid SEQ ID NO: 65 and a beta variable region having the amino acid sequence of SEQ ID NO: 67;
(l) an alpha variable region having the amino acid SEQ ID NO: 69 and a beta variable region having the amino acid sequence of SEQ ID NO: 71;
(m) an alpha variable region having the amino acid SEQ ID NO: 73 and a beta variable region having the amino acid sequence of SEQ ID NO: 75;
(n) an alpha variable region having the amino acid SEQ ID NO: 77 and a beta variable region having the amino acid sequence of SEQ ID NO: 79;
(o) an alpha variable region having the amino acid SEQ ID NO: 81 and a beta variable region having the amino acid sequence of SEQ ID NO: 83; or
(p) an alpha variable region having the amino acid SEQ ID NO: 85 and a beta variable region having the amino acid sequence of SEQ ID NO: 87.

In some embodiments, the nucleic acid sequences encoding the alpha and beta regions of an anti-HA-2 TCR are provided on a single vector or expression cassette and expressed from a single promoter. In some embodiments, the alpha and beta TCR chains are encoded on a single transcript and separated by a 2A element (e.g., a p2A or T2A element).

Also described are lentiviral vectors suitable for use in transducing a T cell to insert a nucleic acid encoding a heterologous anti-HA-1 TCR. In some embodiments, the lentiviral vector comprises a nucleic acid sequence encoding SEQ ID NO: 20 or SEQ ID NO: 22; a nucleic acid sequence having at least 75% identity to SEQ ID NO: 19, SEQ ID NO: 21, or SEQ ID NO: 23 and encoding the amino acid sequence of SEQ ID NO: 20 or SEQ ID NO: 22; or a nucleic acid sequence comprising SEQ ID NO: 19, SEQ ID NO: 21, or SEQ ID NO: 23.

Also described are engineered T cells expressing a heterologous anti-HA-1 TCR. In the embodiments, the engineered T cell comprises a nucleic acid sequence encoding SEQ ID NO: 20 or SEQ ID NO: 22; a nucleic acid sequence having at least 75% identity to SEQ ID NO: 19, SEQ ID NO: 21, or SEQ ID NO: 23 and encoding the amino acid sequence of SEQ ID NO: 20 or SEQ ID NO: 22; or a nucleic acid sequence comprising SEQ ID NO: 19, SEQ ID NO: 21, or SEQ ID NO: 23.

### BRIEF DESCRIPTIONS OF THE DRAWINGS

FIG. 1A. Diagram illustrating exemplary embodiments of TCR expression cassettes.

FIG. 1B. Diagram illustrating an exemplary embodiment of lentiviral vector for inserting a nucleic acid encoding a heterologous TCR expression into a T cell.

FIG. 2. Graph illustrating knockout efficiency in T cells activate using OKT3, OKT3+activating anti-CD28 antibodies, and TRANSACT™

FIG. 3. Graph illustrating percent transduced T cells (insertion of heterologous TCR) as determined by CD44 expression.

FIG. 4. Graphs illustrating percent LCL224 (top panel, cells expressing “R” antigen) or LCL222 (bottom panel, cells expressing “H” antigen) live cells following coculture with varying ratios of engineered T cells.

FIG. 5. Graph illustrating percent LCL224 (cells expressing “R” antigen) or LCL222 (cells expressing “H” antigen) live cells following coculture with varying ratios of engineered T cells.

FIG. 6. Graph illustrating IL-2 secretion, as detected by ELISpot Cytokine release assay, following coculture of engineered T cells with LCL224 (expressing “R” antigen) or LCL222 (expressing “H” antigen) cells. Engineered T cells were manufactured using T cells from two difference donors.

FIG. 7. Graph illustrating intracellular cytokine staining as determined by flow cytometry in engineered T cells following contact with cells presenting the HA-1 “H” peptide (LC222) or HA-1 “R” peptide (LC224).

FIG. 8. Graph illustrating activation of engineered T cells by LCL cells expressing the indicated HLA and HA-1 epitopes.

FIG. 9. Graph illustrating cytotoxicity of engineered T cells against THP-1 cells (AML cell line) and NALM-6 cells (B-ALL cell line).

FIG. 10. Diagram illustrating an exemplary engineered T cell manufacturing and alloSCT+T cell therapy timeline.

### DETAILED DESCRIPTION

### I. Definitions

The singular forms “a,” “an,” and “the” include plural referents unless the context clearly dictates otherwise. For example, “a” or “an” means “at least one” or “one or more.” It is understood that aspects and variations described herein include “consisting of” and/or “consisting essentially of” aspects and variations.

In general, the term “about” indicates variation in a quantity of a component of a composition not having any significant effect on the activity or stability of the composition. When the specification discloses a specific value for a parameter, the specification should be understood as alternatively disclosing the parameter at “about” that value. All ranges are to be interpreted as encompassing the endpoints in the absence of express exclusions, such as “not including the endpoints”; thus, for example, “within 10-15” or “from 10 to 15” includes the values 10 and 15. Also, the use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings. To the extent that any material incorporated by reference is inconsistent with the express content of this disclosure, the express content controls.

The terms “polypeptide” and “protein” are used interchangeably to refer to a polymer of amino acid residues, and are not limited to a minimum length. Polypeptides, including the provided T cell receptors, antigen binding fragments thereof and other peptides, e.g., linkers, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

A “vector” refers to a nucleic acid (e.g., RNA or DNA) encoding one or more expression products (e.g., peptide (i.e., polypeptide or protein)). A vector may be, but is not limited to, a virus or attenuated virus (viral vector), a plasmid, a linear DNA molecule, an mRNA, a CRISPR RNA, a CISPR system, or a composition comprising the nucleic acid encoding the expression product. A vector is capable of expressing one or more polypeptides in a cell, such a mammalian cell. The vector may comprise one or more sequences necessary for expression of the encoded expression product. A variety of sequences can be incorporated into a vector to alter expression of the coding sequence. The vector may comprise one or more of: a 5′ untranslated region (5′ UTR), an enhancer, a promoter, an intron, a 3′ untranslated region (3′ UTR), a terminator, and a polyA signal operably linked to the DNA coding sequence. A vector may also comprise one or more sequences that alter stability of a messenger RNA (mRNA), RNA processing, or efficiency of translation. Any of the described nucleic acids encoding a TCR or CAR can be part of an expression vector designed to express the TCR or CAR in a cell. A viral vector can be, but is not limited to, an AAV vector, an adenovirus, a retrovirus, a gammaretrovirus, a lentivirus, a vaccinia virus, an alphavirus, or a herpesvirus.

The term “plasmid” refers to a nucleic acid that includes at least one sequence encoding a polypeptide (e.g., an expression vector) that is capable of being expressed in a mammalian cell. A plasmid can be a closed circular DNA molecule. A variety of sequences can be incorporated into a plasmid to alter expression of the coding sequence or to facilitate replication of the plasmid in a cell. Sequences can be used that influence transcription, stability of a messenger RNA (mRNA), RNA processing, or efficiency of translation. Such sequences include, but are not limited to, 5′ untranslated region (5′ UTR), promoter, introns, and 3′ untranslated region (3′ UTR). In some embodiments, plasmids can be transformed into bacteria, such as E. coli. A plasmid can also be packaged into a virus to for a viral vector (e.g., a lentiviral vector).

“Operable linkage” or being “operably linked” refers to the juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

A “promoter” is a DNA regulatory region capable of binding an RNA polymerase in a cell (e.g., directly or through other promoter-bound proteins or substances) and initiating transcription of a coding sequence. A promoter may comprise one or more additional regions or elements that influence transcription initiation rate, including, but not limited to, enhancers. A promoter can be, but is not limited to, a constitutively active promoter, a conditional promoter, an inducible promoter, or a cell-type specific promoter.

A “translation modification element” enables translation of two or more genes from a single transcript. Translation modification elements include Internal Ribosome Entry Sites (IRES), which allow for initiation of translation from an internal region of an mRNA, and 2A peptides, derived from picornavirus, which cause the ribosome to skip the synthesis of a peptide bond at the C-terminus of the element. Incorporation of a translation modulating element results in co-expression of two or more polypeptide from a single multicistronic mRNA. 2A modulators include, but are not limited to, P2A, T2A, E2A or F2A. 2A modulators contain a PG/P cleavage site.

A “heterologous” sequence is a sequence which is not normally present in a cell, genome, or gene in the genetic context in which the sequence is currently found. A heterologous sequence can be a sequence derived from the same gene (e.g., a different allele) and/or cell type, but introduced into the cell or a similar cell in a different context, such as on an expression vector or in a different chromosomal location or with a different promoter. A heterologous sequence can be a sequence derived from a different gene or species than a reference gene or species. A heterologous sequence can be from a homologous gene from a different species, from a different gene in the same species, or from a different gene from a different species. For example, a regulatory sequence may be heterologous in that it is linked to a different coding sequence relative to the native regulatory sequence.

A “CRISPR system” comprises a guide RNA, either as a crRNA and a tracrRNA (dual guide RNA) or an sgRNA, and an RNA-guided DNA endonuclease. The guide RNA directs sequence-specific binding of the RNA-guided DNA endonuclease to a target sequence. In some embodiments, the RNA-guided DNA endonuclease contains a nuclear localization sequence. In some embodiments, the CRISPR system further comprises one or more fluorescent proteins and/or one or more endosomal escape agents. In some embodiments, the gRNA and RNA-guided DNA endonuclease are provided in a complex. In some embodiments, the gRNA and RNA-guided DNA endonuclease are provided in one or more expression constructs (CRISPR constructs) encoding the gRNA and the RNA-guided DNA endonuclease. Delivery of the CRISPR construct(s) to a cell results in expression of the gRNA and RNA-guided DNA endonuclease in the cell. The CRISPR system can be, but is not limited to, a CRISPR class 1 system, a CRISPR class 2 system, a CRISPR/Cas system, a CRISPR/Cas9 system, a CRISPR/zCas9 system and a CRISPR/Cas3 system.

The term “CRISPR RNA (crRNA)” has been described in the art (e.g., in Makarova et al. (2011) Nat Rev Microbiol 9:467-477; Makarova et al. (2011) Biol Direct 6:38; Bhaya et al. (2011) Annu Rev Genet 45:273-297; Barrangou et al. (2012) Annu Rev Food Sci Technol 3:143-162; Jinek et al. (2012) Science 337:816-821; Cong et al. (2013) Science 339:819-823; Mali et al. (2013) Science 339: 823-826; and Hwang et al. (2013) Nature Biotechnol 31:227-229). A crRNA contains a sequence (spacer sequence or guide sequence) that hybridizes to a target sequence in the genome. A target sequence can be any sequence that is unique compared to the rest of the genome and is adjacent to a protospacer-adjacent motif (PAM).

A “trans-activating CRISPR RNA” (tracrRNA) is an RNA species that facilitates binding of the RNA-guided DNA endonuclease (e.g., Cas) to the guide RNA.

A “protospacer-adjacent motif” (PAM) is a short sequence recognized by the CRISPR complex. The precise sequence and length requirements for the PAM differ depending on the CRISPR system used, but PAMs are typically 2-5 base pair sequences adjacent the protospacer (i.e., target sequence). Non-limiting examples of PAMs include NGG, NNGRRT, NN[A/C/T]RRT, NGAN, NGCG, NGAG, NGNG, NGC, and NGA.

“Engineered,” “genetically modified,” or simply “modified” cells are cells in which one or more endogenous genes have been modified or disrupted and/or an exogenous (e.g., heterologous) nucleic (such as a TCR) has been introduced and incorporated into the genome of the cell. Engineered cells are distinguishable from naturally occurring cells, which do not a genetic modification (e.g., disruption of one or more endogenous genes and/or insertions of one or more heterologous nucleic acid sequences).

Knock out (also known as gene deletion or gene inactivation) refers to genetic engineering or modification that involves the targeted removal or inactivation of a specific gene within an organism's genome. Knock out can be done through a variety of methods, including homologous recombination, CRISPR-Cas9, and TALENs.

As used herein, “percent (%) amino acid sequence identity” and “percent identity” when used with respect to an amino acid sequence (reference polypeptide sequence) is defined as the percentage of amino acid residues in a candidate sequence (e.g., a subject T cell receptor or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

An amino acid substitution may include replacement of one amino acid in a polypeptide with another amino acid. Amino acid substitutions may be introduced into a TCR or antigen binding fragment thereof, of interest and the products screened for a desired activity, e.g., retained/improved antigen binding, decreased immunogenicity, or improved cytolytic activity.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro; and
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

A cell or population of cells is “positive” (+) for a particular marker (e.g., CD8⁺ or CD34⁺) if the detectable marker can be detected on or in the cell. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

“HLA matching” is used to match patients and donors for blood or marrow transplants. If two people share an HLA alleles, they are considered HLA-matched for that HLA allele. HLA matching can refer to matching of one or more HLA alleles. HLA (human lymphocyte antigen) is type of molecule found on the surface of most cells in the body. HLAs play an important part in the body's immune response to foreign substances. HLAs corresponding to major histocompatibility complex (MHC) class I include HLA-A, HLA-B, and HLA-2. HLAs corresponding to MHC class II include HLA-DR, HLA-DQ, and HLA-DP. A subject has two alleles for each HLA. An HLA 10/10 match indicates the donor and the recipient share two alleles for each of the HLA-A, HLA-B, HLA-C, and HLA-DR, and either the HLA-DP locus or the HLA-DQ locus. An HLA 12/12 match indicates the donor and the recipient share two alleles for each for the HLA-A, HLA-B, HLA-C, HLA-DR, HLA-DQ, and HLA-DP loci. An HLA 11/12 match indicates the donor and the recipient share a single mismatch at the HLA-DQ or HLA-DP locus in the graft-versus-host direction (heterozygous recipient/homozygous donor).

A “hematopoietic restricted miHA” comprises an antigen that is expressed on cells of hematopoietic origin, e.g., white blood cells, but is not expressed or is expressed at very low levels in other cells (cell other than those of hematopoietic origin).

The terms “treat,” “treatment,” and the like, mean the methods or steps taken to provide relief from, or amelioration or alleviation of the number, severity, adverse effect, and/or frequency of one or more symptoms or pathological consequences of a disease, disorder, or condition in a subject. Treating can include inhibiting the disease, disorder or condition, e.g., impeding its progress; and relieving the disease, disorder, or condition, e.g., causing regression of the disease, disorder and/or condition. Treatment can also mean prolonging survival as compared to expected survival in the absence of treatment. Treatment can be therapeutic in terms of a partial or complete cure of a disease, condition, symptom or adverse effect attributed to the disease, disorder, or condition. The term treatment can include: (a) preventing the disease from occurring in a subject who may be predisposed to the disease but has not yet been diagnosed as having it; (b) inhibiting the disease, i.e., arresting its development; and (c) relieving the disease, i.e., mitigating or ameliorating the disease and/or its symptoms or conditions.

An “effective dose” or “effective amount” of an agent, e.g., engineered T cells a pharmaceutical formulation containing the engineered T cells, in the context of administration, refers to an amount effective, at dosages/amounts and for periods of time necessary, to achieve a desired result, such as a therapeutic result.

A treatment or therapy, such as immunosuppressive therapy, can be prophylactic in terms of preventing or partially preventing a condition or disease, or a symptom of a condition of the disease, such as GVHD. Prophylactic administration of a therapy includes administration of the therapy to a subject prior to the occurrence of a condition or disease, prior to appearance or detection of a symptom of a condition or disease, or prior to diagnosis of a condition or disease in the subject.

### II. Engineered T Cells

Described of methods of generating engineered T cells. The methods provide for rapid engineering and expansion of T cells expressing a heterologous TCR or CAR. Using the described methods, engineered T cells suitable for administration to a recipient subject can be generated in 21 days or less. The engineered T cells are suitable for use by themselves in T cell therapy or in combination with other treatments, such as, but not limited to alloSCT. In some embodiments, the described methods are used to generate allogeneic, HLA-matched, TCR-edited T-cells. In some embodiments, the described methods are used to generate allogeneic, HLA-matched, CAR T-cells. In some embodiments, the engineered T cells express a heterologous TCR or CAR that binds to (has affinity for) a target antigen that is expressed in a recipient subject but is not expressed in a donor subject. The recipient can be homozygous or heterozygous for the gene that encodes/expresses the target antigen. In some embodiments, the engineered T cells express a heterologous TCR that binds to (has affinity for) a miHA antigen. In some embodiments, the engineered T cells express a heterologous TCR that binds to (has affinity for) a miHA antigen, wherein the miHA antigen is a hematopoietic restricted miHA antigen. In some embodiments, the engineered T cells express a heterologous TCR that binds to (has affinity for) a peptide epitope expressed on the surface of a cancer cell and/or a cell associated with a hematological ailment. In some embodiments, the engineered T cells express a heterologous TCR that binds to (has affinity for) a peptide epitope expressed on the surface of a cancer cell and/or a cell associated with a hematological ailment in the context of an MHC molecule.

In some embodiments, the methods of generating engineered T cells comprise:
(a) Collecting an apheresis product from a donor subject (or having an apheresis product collected from the donor subject);
(b) Isolating CD8⁺ T cells from the apheresis product;
(c) Activating the CD8⁺ T cells;
(d) Knocking out the endogenous TCR in the CD8⁺ T cells;
(e) Inserting a nucleic acid sequence encoding a heterologous TCR (or CAR) to form engineered T cells expressing the heterologous TCR (or CAR); and
(f) Expanding the engineered T cells.

In some embodiments, PBMCs are isolate from the apheresis product prior to isolating the T cells.

In some embodiments, the methods of generating engineered T cells comprise:
(a) Collecting an apheresis product from a donor subject (or having an apheresis product collected from the donor subject);
(b) Activating the CD8⁺ T cells in the apheresis product;
(c) Isolating activated CD8⁺ T cells from the apheresis product;
(d) Knocking out the endogenous TCR in the CD8⁺ T cells;
(e) Inserting a nucleic acid sequence encoding a heterologous TCR (or CAR) to form engineered T cells expressing the heterologous TCR (or CAR); and
(f) Expanding the engineered T cells.

In some embodiments, PBMCs are isolate from the apheresis product prior to activating the T cells.

In some embodiments, the methods of generating engineered T cells comprise:
(a) Collecting an apheresis product from a donor subject (or having an apheresis product collected from a donor subject);
(b) Isolating CD8⁺ T cells from the apheresis product;
(c) Activating the CD8⁺ T cells;
(d) Inserting a nucleic acid sequence encoding a heterologous TCR (or CAR) to form engineered T cells expressing the heterologous TCR (or CAR); and
(e) Expanding the engineered T cells.

In some embodiments, PBMCs are isolate from the apheresis product prior to isolating the CD8⁺ T cells.

In some embodiments, the methods of generating engineered T cells comprise:
(a) Collecting an apheresis product from a donor subject (or having an apheresis product collected from a donor subject);
(c) Activating the CD8⁺ T cells in the apheresis product;
(b) Isolating activated CD8⁺ T cells from the apheresis product;
(c) Inserting a nucleic acid sequence encoding a heterologous TCR (or CAR) to form engineered T cells expressing the heterologous TCR (or CAR); and
(d) Expanding the engineered T cells.

In some embodiments, PBMCs are isolate from the apheresis product prior to activating the CD8⁺ T cells.

An apheresis product comprises white blood cells or PBMCs obtained from a subject by performing apheresis or leukapheresis.

Collecting an apheresis product from donor subject can be done using methods available in the art for apheresis. Collecting PBMCs from a donor subject can be done using methods available in the art for collection of PBMCs. Such methods include, but are not limited to, apheresis and leukapheresis. In some embodiments, the apheresis product is collected from a healthy donor subject using apheresis on day −1. In some embodiments, the donor subject is administered a mobilizing agent to prior to the apheresis. In some embodiments, the donor subject is not administered a mobilizing agent prior to the apheresis. The donor subject can be, but is not limited to, a healthy donor subject, a healthy allogeneic donor subject, or a subject to be treated with the engineered T cells (recipient subject, i.e., autologous donor subject). In some embodiments, the donor subject is HLA-matched to the recipient subject. In some embodiments, the HLA-matched donor subject is HLA-A, HLA-B, and HLA-C matched to the recipient subject. In some embodiments, the donor subject is an HLA 10/10 match to the recipient subject. In some embodiments, the donor subject is a related donor subject that is an HLA 10/10 match to the recipient subject. In some embodiments, the donor subject is a unrelated donor subject that is an HLA 10/10 match to the recipient subject. In some embodiments, the donor subject is an HLA 11/12 match to the recipient subject. In some embodiments, the donor subject is a related donor subject that is an HLA 11/12 match to the recipient subject. In some embodiments, the donor subject is an unrelated donor subject that is an HLA 11/12 match to the recipient subject. In some embodiments, the donor subject is an HLA 12/12 match to the recipient subject. In some embodiments, the donor subject is a related donor subject that is an HLA 12/12 match to the recipient subject. In some embodiments, the donor subject is an unrelated donor subject that is an HLA 11/12 match to the recipient subject. In some embodiments, the donor subject does not express an antigen (target antigen) that is expressed by the recipient subject. In some embodiments, the donor subject does not express a hematopoietic restricted miHA antigen that is expressed by the recipient subject. In some embodiments, the donor subject does not express an antigen that is recognized by the heterologous TCR to be expressed in the engineered T cells derived from the donor subject. The heterologous TCR or CAR expressed by the engineered T cells has affinity for the antigen that is expressed by the recipient subject but not by the donor subject. The recipient subject can be homozygous or heterozygous for the gene that encodes/expresses the target antigen.

CD8⁺ T cells (including activated CD8⁺ T cell) can be isolated from an apheresis product using methods available in the art for isolating, purifying, or selecting CD8⁺ T cells, such as from peripheral blood. Such methods include, but are not limited to, cell sorting, or fluorescence assisted cell sorting. The CD8⁺ T cell can be, but are not limited to, a naïve T cell (T_{naïve} cell), an effector T cell, an effector memory T cell (Tₑₘ cell), a CD4⁺/CD8⁺ T cell, a helper T cell, a CD4⁺ T cell, a CD4⁺ helper T cell, a Th1 T cell, a Th2 T cell, a cytotoxic T cell, a memory T cell, a central memory T cell (T_{cm} cell), a regulatory T cell, an αβ T cell, a T6 T cell, or a combination thereof. In some embodiments, CD8⁺ T cells are isolated from the apheresis product before activation. In some embodiments, CD8⁺ T cells are isolated from the apheresis product following activation. In some embodiments, the apheresis product comprises PBMCs or purified PBMCs. In some embodiments, the CD8⁺ T cells are isolated from the apheresis product on day 0 of the manufacturing process. In some embodiments, activated CD8⁺ T cells are isolated from the apheresis product on day 1 of the manufacturing process.

Activating the CD8⁺ T cells can be done using methods available in the art for activating CD8⁺ T cells. Such methods include, but are not limited to, contacting the CD8⁺ T cells (or PBMCs containing CD8⁺ T cells) with CD3 and/or CD28 agonists. In some embodiments, activating the CD8⁺ T cells comprises contacts the T cells with CD3 and CD28 agonists. In some embodiments, the CD3 and CD28 agonists are immobilized. CD3 agonists include, but are not limited to, activating anti-CD3 antibodies. CD28 agonists include, but are not limited to, activating anti-CD28 antibodies. The activating anti-CD3 antibody can be any anti-CD3 antibody that interferes with or reduces interaction between CD3 and a TCR. The anti-CD3 antibody can be, but is not limited to, an OKT3 antibody, a 17A2 antibody, a 145-2C11 antibody, or a UCHT1 antibody. In some embodiments the anti-CD3 antibody is an OKT3 antibody. The activating anti-CD28 antibody can be any antibody that acts as a co-stimulator in activating T cells in vitro.

In some embodiments, CD8⁺ T cells are activated by contacting the apheresis product or PBMCs with soluble anti-CD3 antibody. In some embodiments, the apheresis product or PBMCs are contacting with soluble anti-CD3 antibody starting on day 0 of the manufacturing process. In some embodiments, the apheresis product or PBMCs are incubated with soluble anti-CD3 antibody for about 16 to about 36 hours In some embodiments, apheresis product or PBMCs are incubated with soluble anti-CD3 antibody for about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, or about 36 hours. In some embodiments, apheresis product or PBMCs are incubated with soluble anti-CD3 antibody for 24±4 hours. In some embodiments, apheresis product or PBMCs are incubated with soluble anti-CD3 antibody for about 20 to about 28 hours. In some embodiments, apheresis product or PBMCs are incubated with soluble anti-CD3 antibody until at least 50%, at least 60%, at least 70%, at least 80%, or at least 90% of the cells are activated T cells. The percentage of activated T cells can be determined by methods available in the art, such as, but not limited to, analysis of CD69 expression. In some embodiments, apheresis product or PBMCs are incubated with soluble anti-CD3 antibody until at least 50%, at least 60%, at least 70%, at least 80%, or at least 90% of the cells are CD69⁺ T cells.

In some embodiments, CD8⁺ T cells are isolated from the apheresis product PBMCs prior to activation. In some embodiments, the apheresis product or PBMCs are collected from a donor subject that has not been administered a mobilizing agent to prior to the apheresis. The CD8⁺ T cells can be isolated from the apheresis product or PBMCs on day 0 of the manufacturing process. The isolated CD8⁺ T cells are activated by contacting (incubating) the CD8⁺ T cells with CD3 and CD28 agonists. In some embodiments, the CD3 and CD28 agonists are immobilized. CD3 agonists include, but are not limited to, activating anti-CD3 antibodies. CD28 agonists include, but are not limited to, activating anti-CD28 antibodies. The CD8⁺ T can be contacted with the CD3 and CD28 agonists starting on day 0. In some embodiments, the isolated CD8⁺ T cells are contacted with the CD3 and CD28 agonists overnight (i.e., about 12 to about 24 hours). The CD3 and CD28 agonists may be linked to beads (e.g., DYNABEADS™). CD3/CD28 agonist T cell activating reagents include, but are not limited to, TRANSACT™ beads. In some embodiments, CD8⁺ T cells are incubated with the CD3 and CD28 agonists for 12 hours. In some embodiments, CD8⁺ T cells are incubated with the CD3 and CD28 agonists for 14 hours. In some embodiments, CD8⁺ T cells are incubated with the CD3 and CD28 agonists for 16 hours. In some embodiments, CD8⁺ T cells are incubated with the CD3 and CD28 agonists for 18 hours. In some embodiments, CD8⁺ T cells are incubated with the CD3 and CD28 agonists for 19 hours. In some embodiments, CD8⁺ T cells are incubated with the CD3 and CD28 agonists for 20 hours. In some embodiments, CD8⁺ T cells are incubated with the CD3 and CD28 agonists for 21 hours. In some embodiments, CD8⁺ T cells are incubated with the CD3 and CD28 agonists for 22 hours. In some embodiments, CD8⁺ T cells are incubated with the CD3 and CD28 agonists for 23 hours. In some embodiments, CD8⁺ T cells are incubated with the CD3 and CD28 agonists for 24 hours. In some embodiments, CD8⁺ T cells are incubated with the CD3 and CD28 agonists until at least 50%, at least 60%, at least 70%, at least 80%, or at least 90% of the cells are activated T cells. The percentage of activated T cells can be determined by methods available in the art, such as, but not limited to, analysis of CD69 expression. In some embodiments, CD8⁺ T cells are incubated with the CD3 and CD28 agonists until at least 50%, at least 60%, at least 70%, at least 80%, or at least 90% of the cells are CD69⁺ T cells.

In some embodiments, the endogenous TCR is knocked out in the isolated, activated CD8⁺ T cells. Knocking out of the endogenous TCR can be done using methods available in the art for knocking out expressing of an endogenous gene in a mammalian cell. Such methods include, but are not limited to, CRISPR, ARCUS (Precision BioSciences), zinc finger nucleases (ZFN), transcription activator-like effector nucleases (TALENs), and meganucleases of microorganisms. Knocking out the endogenous TCR can (a) eliminate mispairing of an endogenous TCR chain with a chain of the introduced heterologous TCR, (b) reduce potential activity of the endogenous TCR against a host cells, which could contribute to or cause GVHD, or (c) alleviate the potential for competition between the endogenous and exogenous TCR. Knocking out the endogenous TCR comprises any modification that results in the T cell not expressing a functional endogenous TCR. The modification can be a deletion of all or a portion of the endogenous TCR gene, or introduction of a mutation (e.g., a frame shift or nonsense mutation) or insertion into the endogenous TCR gene such that the functional endogenous TCR is not expressed. In some embodiments, the modification results neither the α or β chain (or γ or 6 chain) of the endogenous TCR being expressed. In some embodiments, the modification results in neither the α or β chain constant regions (or γ or δ chain constant regions) of the endogenous TCR being expressed. In some embodiments, the modification results in no expression of any portion of the endogenous TCR protein being expressed. In some embodiments, the endogenous TCR is knocked out in the isolated, activated CD8⁺ T cells on day 1 of the manufacturing process.

In some embodiments, knocking out the endogenous T cell receptor in the isolated T cells is performed using a CRISPR system. Both the TCR α chain (TRAC gene) and the TCR β (TRBC1 and TRBC2 genes) chain can be knocked out using CRISPR systems. The CRISPR system comprises an RNA-guided DNA endonuclease enzyme and a guide RNA. The RNA-guided DNA endonuclease enzyme can be, but is not limited to, a Cas9 enzyme (e.g., SpCas9). In some embodiments, two guide RNAs are used, one to target the TRAC gene (TRAC guide RNA) and one to target the TRBC genes (TRBC guide RNA). The Cas9/TRBC guide RNA knocks out both the TRBC1 and TRBC2 genes. The endonuclease and the guide RNA can be provided in a pre-formed ribonucleoprotein (RNP complex). In some embodiments, the CRISPR system comprises a Cas9/guide RNA pre-formulated ribonucleoprotein (RNP). In some embodiments, the CRISPR system comprises two Cas9/guide RNA pre-formulated RNPs. In some embodiments, the CRISPR system creates a double strand break in the coding region of the TCR constant chain. Repair of the break creates a short (1-5 nucleotide) insertions and/or deletions in the genomic sequence. In some embodiments, the CRISPR system can be designed to delete some or up to all of the endogenous gene. The Cas9/guide RNA pre-formulated RNPs can be introduced into the T cell using methods known in the art for introducing a CRISPR RNP into a mammalian cell.

The TRAC guide RNA can be, but is not limited to, 5′-GAGAAUCAAAAUCGGUGAAUGUUUUAGAGCUAGAAAUAGCAAGUUAAAAU AAGGCUAGUCCGUUAUCAACUUGAAAAAGUGGCACCGAGUCGGUGCUUUU-3′ (SEQ ID NO: 1). The TRAC guide RNA can be provided in a modified form comprising:

GmsAmsGmsAAUCAAAAUCGGUGAAUGUUUUAGAGCUAGAAAUAGCAAG
UUAAAAUAAGGCUAGUCCGUUAUCAACUUGAAAAAGUGGCACCGAGUCG
GUGCUUmsU

msUm, wherein U, A, C, G indicates: RNA; m=2′-O-methyl sugar analog, and s=3′ phosphorothioate internucleotide linkages (SEQ ID NO: 1)

The TRBC guide RNA can be, but is not limited to, 5′-CACCCAGAUCGUCAGCGCCGGUUUUAGAGCUAGAAAUAGCAAGUUAAAAUA AGGCUAGUCCGUUAUCAACUUGAAAAAGUGGCACCGAGUCGGUGCUUUU-3′ (SEQ ID NO: 2). The TRBC guide RNA can be provided in a modified form comprising:

CmsAmsCmsCCAGAUCGUCAGCGCCGGUUUUAGAGCUAGAAAUAGCAAG
UUAAAAUAAGGCUAGUCCGUUAUCAACUUGAAAAAGUGGCACCGAGUCG
GUGCUUmsU

msUm, wherein U, A, C, G indicates: RNA; m=2′-O-methyl sugar analog, and s=3′ phosphorothioate internucleotide linkages (SEQ ID NO: 2).

Both the TRAC guide RNA and the TRBC guide RNA are 100 nucleotides (nt) in length and contain the necessary sequence elements for gene editing: the 20-nt target-specific recognition sequence (“the guide”), the canonical 3-nt SpCas9 PAM sequence, and the approximately 80-nt tracrRNA scaffold that interacts with the Cas9 enzyme.

The CRISPR system (e.g., the RNP) can be delivered to the CD8⁺ T cells using methods available in the art for delivering an RNP into a mammalian cell. Such methods include, but are not limited to, electroporation.

In some embodiments, about 1×10⁵ to about 1×10⁸ activated CD8⁺ T cells are modified to knock out the endogenous TCR. In some embodiments, about 60×10⁶ activated CD8⁺ T cells are modified to knock out the endogenous TCR. Knockout efficiency can be >90%.

In some embodiments, the donor T cells are not modified to knock out expression of the endogenous TCR. In such embodiments, the heterologous TCR may be modified to reducing mispairing of the heterologous TCR α or β chain with the endogenous TCR β or a chain or to promote pairing between the heterologous TCR α and β chains.

The isolated CD8⁺ T cells are modified to express a heterologous TCR or CAR by introducing a heterologous nucleic acid sequence (TCR nucleic acid or CAR nucleic acid) encoding and the TCR or CAR to be expressed into the T cells. A heterologous nucleic acid encoding a heterologous TCR (or an antigen binding fragment thereof) or CAR is introduced into the T cell (e.g., via lentiviral transduction). Inserting the heterologous nucleic acid encoding the heterologous TCR (or an antigen binding fragment thereof) or CAR can be performed using methods available in the art for introducing a heterologous nucleic acid sequence into a mammalian cell. The heterologous nucleic acid encoding the heterologous TCR or CAR can be introduced into the T cell prior to knocking out the endogenous TCR, concurrently with knocking out the endogenous TCR, or after knocking out the endogenous TCR. In some embodiments, the heterologous nucleic acid encoding the heterologous TCR or CAR is introduced into the isolated CD8⁺ T cell after knocking out the endogenous TCR. In some embodiments, the heterologous nucleic acid encoding the heterologous TCR or CAR is introduced into the isolated CD8⁺ T cell after knocking out the endogenous TCR on day 1 of the manufacturing process. Introducing the heterologous nucleic acid into the T cell may be done using methods available in the art. For example, the TCR or CAR nucleic acid can be introduced into an isolated CD8⁺ T cell by various transfection or transformation methods known in the art. TCR or CAR nucleic acid can be introduced into an isolate CD8⁺ T cell using a viral vector or a non-viral vector. Methods of introducing a nucleic acid into a cell include, but are not limited to, viral vectors, microinjection, microprojectile bombardment (e.g., gene gun), electroporation, lipofection, and CRISPR (e.g., CRISPR-Cas9) systems, CRISPR knock in (LIFE EDIT), transposon-mediated, ARCUS (Precision BioSciences), zinc finger nucleases (ZFN), transcription activator-like effector nucleases (TALENs), meganucleases of microorganisms.

In some embodiments, inserting a nucleic acid sequence encoding the heterologous TCR or CAR comprises transducing the T cells with a viral vector containing a heterologous nucleic acid sequence encoding the heterologous TCR (or an antigen binding fragment thereof) or CAR. The viral vector can be, but is not limited to, an adeno-associated virus (AAV), an adenovirus, a retrovirus, a gammaretrovirus, a lentivirus, a vaccinia virus, an alphavirus, or a herpesvirus. In some embodiments, the viral vector is a lentivirus vector. In some embodiments, the viral vector is a lentivirus vector and the isolated CD8⁺ T cells are transduced at a multiplicity of infection of about 2.5. About 1×10⁵ to about 1×10⁸ isolated CD8⁺ T cells, or about 60×10⁶ isolated CD8⁺ T cells are transduced to express the heterologous TCR or CAR. In some embodiments, the nucleic acid encoding the heterologous TCR or CAR is inserted into the CD8⁺ T cells within about 24 hours of knocking out the endogenous TCR

In some embodiments, the engineered T cells are further modified to express a cell detection marker and/or a removal marker. A detection marker comprises any gene or protein, which, when expressed in a T cell, provides for detection of modified T cells or can be used to select modified T cells. Detection markers can be used to aid in monitoring transfection/transduction efficiency or to select transfected/transduced T cells. A detection marker can be, but is not limited to, a CD34 polypeptide or a polypeptide recognized by the QBEnd10 monoclonal antibody (e.g., a CD34 epitope) A removal marker comprises a gene or protein which, when expressed in a T cell, can be used to selectively kill or eliminate the T cell. A removal marker can be, but is not limited to, a polypeptide recognized by the Rituximab monoclonal antibody (e.g., a CD20 epitope). In some embodiments, the engineered T cells are further modified to express an RQR8 marker. RQR8 contains a CD34 epitope and two CD20 mimotopes that can be targeted by the EMA-approved anti-CD20 antibody rituximab. Rituximab recognition of the CD20 epitope allows for selective removal of the engineered T cells in vivo. The detection/removal marker can be encoded by the lentivirus used to transduce the heterologous TCR into the donor T cells. A nucleic acid sequence encoding the heterologous TCR can be present on a polycistronic vector that also encodes the detection/removal marker. In some embodiments, a nucleic acid sequence encoding the heterologous TCR is operably linked to a nucleic acid sequence encoding the detection/removal marker. The nucleic acid sequence encoding the heterologous TCR can be linked to the nucleic acid sequence encoding the detection/removal marker via a sequence encoding a 2A element. In some embodiments, the detection marker (e.g., CD34 epitope) is used as a surrogate for potency measurement or as a surrogate marker for determination of the number of transduced cells.

In some embodiments, the engineered T cell contains 5 or fewer copies of the nucleic acid encoding the heterologous TCR or CAR. In some embodiments, the engineered T cell contains fewer than 5 or fewer copies of the nucleic acid encoding the heterologous TCR or CAR. In some embodiments, the engineered T cell contains 1, 2, 3, 4, or 5 copies of the nucleic acid encoding the heterologous TCR or CAR. In some embodiments, sufficient viral particles are used to transduce the isolated CD8⁺ T cells to average about 2 copies of the nucleic acid sequence encoding the heterologous TCR per cell.

In some embodiments, the engineered T cell are further modified. The further modification can be done prior to modifying the T cell to express the heterologous TCR or CAR, simultaneously with modifying the T cell to express the heterologous TCR or CAR, or after modifying the T cell to express the heterologous TCR or CAR, or a combination thereof.

The further modification can add one or more desired functions to the engineered T cells. In some embodiments, the T cells are further modified by introduction of a nucleic acid encoding an additional gene. The T cells can be further modified by introducing into the T cells, one or more nucleic acids encoding one or more of, for example, a secreted cytokine, a cytokine receptor, a gene that enhances fitness, or an antibody-like protein, or combinations thereof. In some embodiments, the T cells are further modified to alter or delete one or more genes normally expressed in the T cell.

Following genetic modification of the T cells to express the heterologous TCR or CAR, the engineered T cells are grown under conditions suitable for expansion of the engineered T cells. The engineered T cells are optionally purified, using the detection marker, to select those CD8⁺ T cells that express the detection marker. In some embodiments, expanding the engineered T cells comprises contacting the engineered T cells with at least one cytokine that targets a common gamma chain (interleukin-2 receptor subunit gamma or IL-2RG) family receptor and is known to stimulate T cell growth/proliferation. In some embodiments, the at least one cytokine comprises IL-2. In some embodiments, the at least one cytokine comprises IL-2 and one ore more of: IL-7, IL-15, IL-21, IL-9 (e.g., IL-2 plus IL-7). The engineered T cells can be incubated with the one or more cytokines starting on day 2 of the manufacturing process. The engineered T cells can be incubated with the one or more cytokines for about 8 to about 14 days. In some embodiments, the engineered T cells are incubated with the one or more cytokines for about 7, about 8, about 9, about 10, about 11 days, or about 12 days. In some embodiments, the engineered T cells are incubated with the one or more cytokines for about 10 days. In some embodiments, the engineered T cells are expanded to provide at least 100 million, at least 200 million, at least 500 million, or at least 1 billion engineered T cells.

The expanded T cells can be assessed by production of cytokines IL-2 and IFN-7 following exposure to cells presenting the target antigen. In some embodiments, the engineered T cells are analyzed using an activation/degranulation marker assay to evaluate the ability of the engineered T cells to express the surface marker CD107a following exposure to cells (e.g., T2 cells) presenting the target antigen. CD107a is a marker of T cell degranulation which is part of the cell killing response. The engineered T cells are incubated with cells (e.g., T2 cells) loaded with the target peptide antigen. As a control, APCs are loaded with the corresponding nontarget peptide antigen or an irrelevant peptide control. Degranulation responses are analyzed by CD107a surface staining and analysis by FACS.

Following expansion, the engineered T cells can be assayed to determine percent viable T cells and/or present T cells expressing the heterologous TCR or CAR. In some embodiments, the engineered T cells are formulated in an appropriate media or solution (e.g., PLASMA-LYTE™, optionally with 5% human serum albumin) at a 2× concentration relative to the concentration to be administered to the recipient subject. The concentration to be administered to the recipient subject can be about 5×10⁶ cells/mL to about 1×10⁷ cells/mL. The engineered T cells at 2× concentration are diluted 1:1 in cryogenic media (to achieve a concentration of 1× engineered T cells). The cryogenic media can have a DMSO concentration of 10% (i.e., final concentration of 5%). The 1× engineered T cells in cryogenic media can be transferred to a cryogenically compatible infusion bag and cryopreserved. The engineered T cells can be cryopreserved using methods available in the art for the cryopreservation of T cells or CAR T cells. Using the described methods, the expanded, engineered T cells can be cryopreserved on one or more of days 8, 9, 10, 11, 12, 13, 14 or day 15 following activating the CD8⁺ T cells. In some embodiments, the engineered T cells are cryopreserved in CS10 media. In some embodiments, the cells are cryopreserved in a controlled rate freezer (CRF).

In some embodiments, the engineered T cells are generated, expanded, and cryopreserved within about 9 to about 15 days of collecting an apheresis product or harvesting PBMCs from a donor subject. In some embodiments, the engineered T cells are generated, expanded, and cryopreserved within about 9, about 10, about 11, about 12, about 13, about 14, or about 15 days of collecting an apheresis product or harvesting PBMCs from a donor subject.

The engineered T cells can be cryopreserved (e.g., in an infusion bag) at a single dose suitable for administration to an intended recipient subject. In some embodiments, the engineered T cells are formulated in an infusion-ready format. A single dose of engineered T cells can be about 1×10⁴ to about 3×10⁶ engineered T cells (viable T cells expressing the heterologous TCR)/kg of recipient. If cryopreserved, the engineered T cells are thawed prior to administration to the recipient subject. The dose of engineered T cells can be calculated as follows:

$$v = w \times x \div [ y \times \% viable cells \times \% viable cells expressing the heterologous TCR or CAR ]$$

wherein
   v=volume T cells in mL
   w=weight of recipient in kg
   x=dose in T cells/kg
   y=concentration T cells (in number of cells/mL)

Optionally, at least two doses are packaged, each in a separate infusion bag, for the intended recipient subject. The at least two doses can be dose and a replacement dose; first dose and at least one second dose; or first dose, at least one second dose, and a replacement dose.

In some embodiments, isolating T cells (e.g., CD8⁺ T cells), knocking out the endogenous TCR, and inserting the nucleic acid encoding the heterologous TCR or CAR are performed within 36-72 hours of the apheresis product being collected. In some embodiments, isolating T cells (e.g., CD8⁺ T cells), knocking out the endogenous TCR, and inserting the nucleic acid encoding the heterologous TCR or CAR are performed within 24-48 hours of the apheresis product being collected. In some embodiments, the nucleic acid encoding the heterologous TCR or CAR is inserted into the CD8⁺ T cells within about 24 hours of knocking out the endogenous TCR

In some embodiments, the engineered T cells have a generally stem-cell like phenotype. In some embodiments, the thawed engineered T cells generally have a central memory (Tcm) T cell, effector memory (Tem) T cell, or stem memory (Tscm) T cell phenotype. In some embodiments, about 10% to about 90% of the engineered T cells have a stem-cell like phenotype. In some embodiments, at least 20%, at least 30%, at least 40%, at least 50%, or at least 50%, at least 60%, at least 70%, or at least 80% of the engineered T cells have a stem-cell like phenotype. In some embodiments, the engineered T cells generally have a central memory (Tcm) T cell, effector memory (Tem) T cell, or stem memory (Tscm) T cell phenotype. In some embodiments, about 10% to about 90% of the engineered T cells have a Tcm, Tem, or Tscm phenotype. In some embodiments, at least 20%, at least 30%, at least 40%, at least 50%, or at least 50%, at least 60%, at least 70%, or at least 80% of the engineered T cells have a Tcm, Tem, or Tscm phenotype. In some embodiments, at least 50% of the engineered T cells have a stem-cell like phenotype. In some embodiments, greater than 50% of the engineered T cells have a Tcm, Tem, or Tscm phenotype. In some embodiments, at least 50% of the cryopreserved engineered T cells have a stem-cell like phenotype after thawing. In some embodiments, greater than 50% of the cryopreserved engineered T cells have a Tcm, Tem, or Tscm phenotype after thawing.

In some embodiments, the engineered T cells are analyzed for the ability to lyse cells presenting the target antigen using a killing assay. Engineered T cells are incubated with a mixture of fluorescent-tag labeled cells (e.g., T2 cells) differentially loaded with target and control peptides to allow on-target and off-target cytotoxicity. Fluorescently tagged cells are loaded with the target miHA antigen (e.g., HA-1 “H” peptide) or a nontarget miHA antigen (e.g., HA-1 “R” peptide) or an irrelevant peptide control. Cell killing is analyzed by FACS. For engineered T cells expressing, for example, an anti-HA-1 “H” antigen TCR, the target cells can be T2 cells (HA-1⁻/HLA-A*:02:01⁺) loaded with target HA-1 “H” peptide. As a control, T2 cells are loaded with the nontarget HA-1 “R” peptide or an irrelevant peptide control. IL-2 and IFN-7 responses of the T cells is be followed by intracellular cytokine staining and analysis by FACS.

The engineered T cells kill (are cytotoxic toward) cells expressing an antigen recognized by the heterologous TCR or CAR. In some embodiments, the engineered T cells exhibit cytotoxicity against cells expressing both the target antigen and the cognate HLA for the target antigen.

In some embodiments, the engineered T cells kill (are cytotoxic toward) cells expressing an antigen recognized by the heterologous TCR or CAR at an Effector (T cell):Target ratio (antigen expressing cell) about 1:1. In some embodiments, the engineered T cells kill (are cytotoxic toward) cells expressing an antigen recognized by the heterologous TCR or CAR at an Effector (T cell):Target (antigen expressing cell) ratio less than 1:1. In some embodiments, the engineered T cells kill (are cytotoxic toward) cells expressing an antigen recognized by the heterologous TCR or CAR at an Effector (T cell):Target ratio (antigen expressing cell) of less than 0.5:1 or less. In some embodiments, the engineered T cells kill (are cytotoxic toward) cells expressing an antigen recognized by the heterologous TCR or CAR at an Effector (T cell):Target ratio (antigen expressing cell) of about 0.1:1 or less. In some embodiments, the engineered T cells kill (are cytotoxic toward) cells expressing an antigen recognized by the heterologous TCR or CAR at an Effector:Target ratio of less than 0.1:1, less than 0.2:1, less than 0.3:1, less than 0.4:1, less than 0.5:1, or less than 1:1. In some embodiments, the engineered T cells maintain at least 50%, at least 60%, at least 70%, at least 80%, or at least 90%, of their cytolytic potency over a period of at least 20 days. In some embodiments, the engineered T cells maintain at least 50%, at least 60%, at least 70%, at least 80%, or at least 90% of their cytolytic potency after re-stimulation.

In some embodiments, thawed cryopreserved engineered T cells kill (are cytotoxic toward) cells expressing an antigen recognized by the heterologous TCR or CAR at an Effector:Target ratio of about 1:1. In some embodiments, the thawed cryopreserved engineered T cells kill (are cytotoxic toward) cells expressing an antigen recognized by the heterologous TCR or CAR at an Effector:Target ratio of less than 1:1. In some embodiments, the thawed cryopreserved engineered T cells kill (are cytotoxic toward) cells expressing an antigen recognized by the heterologous TCR or CAR at an Effector:Target ratio of less than 0.5:1. In some embodiments, the thawed cryopreserved engineered T cells kill (are cytotoxic toward) cells expressing an antigen recognized by the heterologous TCR or CAR at an Effector:Target ratio of about 0.1:1 or less. In some embodiments, the thawed cryopreserved engineered T cells kill (are cytotoxic toward) cells expressing an antigen recognized by the heterologous TCR or CAR at an Effector:Target ratio of less than 0.1:1, less than 0.2:1, less than 0.3:1, less than 0.4:1, less than 0.5:1, or less than 1:1. In some embodiments, the thawed cryopreserved engineered T cells maintain at least 50%, at least 60%, at least 70%, at least 80%, or at least 90%, of their cytolytic potency for at least an additional 20 days after thawing.

In some embodiments, the engineered T cells made using the described methods have an improved Effector/Target ratio in killing assays. Such killing assays comprise incubating target cells with varying ratios of effector cells (T cells) and determining the ratio of effector cells needed to kill the target cells. Increased longevity can be measured by analyzing cytotoxicity at various times. In some embodiments, the engineered T cells made using the described methods maintain a high level of cytotoxicity over time and multiple exposures to target cells (re-stimulation). In some embodiments, the engineered T cells maintain at least 50%, at least 60%, at least 70%, at least 80%, or at least 90% of their cytolytic potency over a period of a least 20 days. In some embodiments, the engineered T cells maintain at least 50%, at least 60%, at least 70%, at least 80%, or at least 90% of their cytolytic potency over a period of a least 20 days and at least three exposures to target cells.

### III. TCR or CAR

The heterologous TCR can be, but is not limited to, an αβ TCR or an antigen binding fragment thereof, or a 76 TCR or an antigen binding fragment thereof. In some embodiments, the TCR is an allogeneic TCR.

The CAR can be, but is not limited to, a first generation CAR (a CAR having a single signaling domain, such as a CD3ζ signaling domain), a second generation CAR (a CAR having a co-stimulatory domain), a third generation CAR (a CAR having multiple co-stimulatory domains), a fourth generation CAR (TRUCK or armored CAR), or dual-antigen receptor CAR.

In some embodiments, the TCR is a molecule that contains an alpha chain comprising a Vα region and a beta chain comprising a Vβ region (also known as TCRα and TCRβ, respectively) or a gamma chain comprising a Vγ region and a delta chain comprising a VS region (also known as TCRγ and TCRδ, respectively), or antigen-binding portions thereof, which is capable of specifically binding to an antigen, e.g., a peptide antigen or peptide epitope bound to an MHC molecule. In some embodiments, the TCR is in the αβ form (e.g., is an αβ TCR). In some embodiments, the TCR is in the γδ form (e.g., is an γδ TCR). Typically, TCRs that exist in αβ or γδ forms are generally structurally similar, but T cells expressing them may have distinct anatomical locations or functions. Generally, a TCR is found on the surface of T cells where it is generally responsible for recognizing antigens, such as peptides bound to MHC molecules.

In some embodiments, the TCR is an intact or full-length TCR, such as a TCR containing a full length α chain and a full length β chain, or a TCR containing a full length γ chain and a full length δ chain. In some embodiments, an antigen-binding portion of a TCR is less than a full-length TCR provided that it binds to a specific peptide bound in an MHC molecule, such as it binds to an MHC-peptide complex. In some embodiments, an antigen-binding portion or fragment of a TCR can contain only a portion of the structural domains of a full-length or intact TCR, but yet is able to bind the peptide epitope, such as MHC-peptide complex, to which the full-length TCR binds. In some cases, an antigen-binding portion contains the variable domains of a TCR, such as a Vα region and a Vβ region of a TCR, or a Vγ region and a Vδ region of a TCR provided herein provided that that antigen-binding portion is sufficient to form a binding site for binding to a specific MHC-peptide complex.

The heterologous TCR can be derived from a T cell isolated from an autologous subject or a donor subject. In some embodiments, the heterologous TCR is derived from a tumor infiltrating T cell from an autologous subject.

The heterologous TCR or CAR can be, but is not limited to, an anti-miHA antigen TCR or CAR, an anti-miHA HA-1 TCR or CAR, an anti-viral antigen TCR or CAR, or an anti-tumor neoantigen TCR or CAR. In some embodiments, the engineered T cells express a heterologous TCR or CAR against a miHA that is relatively restricted to hematopoietic cells.

In some embodiments, the TCR or antigen-binding fragment thereof recognizes a peptide epitope of a minor histocompatibility antigen (miHA) HA-1 in the context of an MHC molecule. In some embodiments, the MHC molecule is a human leukocyte antigens (HLA)-A molecule. In some embodiments, the HLA-A molecule is of serotype HLA-A*02:01. In some embodiments, the HLA-A molecule is of serotype HLA-A*02:06. In some embodiments, the peptide epitope of HA−1 is VLHDDLLEA or VLRDDLLEA.

### IV. Nucleic Acids Sequences

Described are lentivirus vectors carrying an expression cassette for expression of a heterologous TCR and optionally a marker in a CD8⁺ T cell. The described lentivirus vectors are used to transduce a CD8⁺ T cell to express the heterologous TCR. Plasmids for generating such vectors are also provided. The lentivirus vector can be a third generation, replication-incompetent and self-inactivating vector.

In some embodiments, the expression cassette encoding the TCR comprises nucleic acid sequences encoding an alpha (α) chain and a beta (β) chain or a gamma (γ) chain and a delta (δ) chain. The nucleic encoding the α (or γ) chain and the nucleic acid encoding the β (or δ) chain can be expressed from separate promoters or a single promoter. The nucleic encoding the α (of γ) chain and the nucleic acid encoding the β (or δ) chain can be expressed from a single promoter. In some embodiments, the nucleotide sequence encoding the TCR α (or γ) chain and the nucleotide sequence encoding the TCR β (or δ) chain are separated by a nucleic acid sequence encoding a peptide sequence that causes ribosome skipping. The peptide sequence that causes ribosome skipping can be a 2A peptide. The 2A element can be, but is not limited to, P2A peptide or a T2A peptide. In some embodiments, the nucleotide sequence encoding the TCR α (or γ) chain and the nucleotide sequence encoding the TCR β (or δ) chain are separated by an internal ribosome entry site (IRES).

In some embodiments, the vector encoding the TCR expression cassette contains a single promoter that drives the expression of one or more nucleotide sequences encoding one or more polypeptides. In some embodiments, such vectors can be multicistronic (e.g., bicistronic or tricistronic, see e.g., U.S. Pat. No. 6,060,273). For example, in some embodiments, transcription units can be engineered as a bicistronic unit containing an IRES, which allows coexpression of gene products (e.g., encoding an α chain and a β chain of a TCR) by a message from a single promoter. Alternatively, in some cases, a single promoter may direct expression of an RNA that contains, in a single open reading frame (ORF), two or three genes (e.g., encoding an α chain and a β chain of a TCR) separated from one another by sequences encoding a self-cleavage peptide (e.g., 2A peptide) or a protease recognition site (e.g., furin). The ORF thus encodes a single polyprotein, which, either during or after translation, is cleaved into the individual proteins. In some cases, the peptide, such as 2A peptide, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Examples of 2A cleavage peptides, including those that can induce ribosome skipping, are Thosea asigna virus (T2A), porcine teschovirus-1 (P2A), equine rhinitis A virus (E2A), and 2A sequences from the foot-and-mouth disease virus (F2A) as described in U.S. Patent Publication No. 2007/0116690.

In some embodiments, the nucleic acid sequence encoding the α or γ chain and the nucleotide sequence encoding the β or δ chain are present in any order, separated by the nucleotide sequence encoding a peptide sequence that causes ribosome skipping. For example, in some embodiments, the nucleotide sequence comprises a nucleic acid sequence encoding a β or δ chain, a nucleic acid sequence encoding a peptide sequence that causes ribosome skipping, e.g., a P2A sequence, and a nucleic acid sequence that encodes an α or γ chain, in that order. In other embodiments, the nucleotide sequence contains a nucleic acid sequence that encodes an α or γ chain, a nucleic acid sequence that encodes a peptide sequence that causes ribosome skipping, e.g., a P2A sequence, and a nucleic acid sequence that encodes a β or δ chain, in that order.

In some embodiments, the nucleic acid encoding the heterologous TCR or CAR is codon optimized.

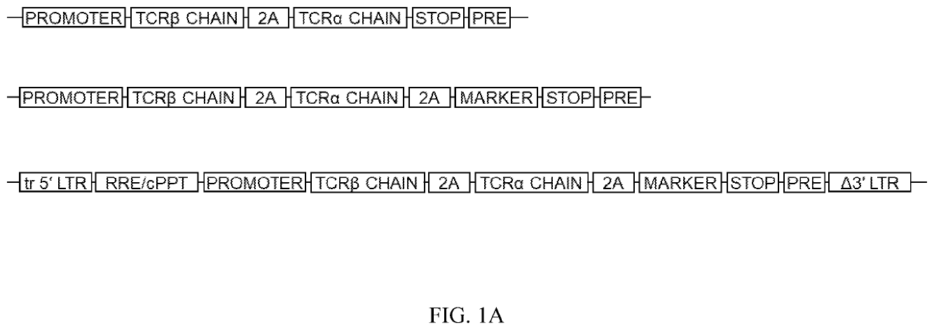

Diagrams illustrating exemplary TCR expression cassettes are shown in FIG. 1A and FIG. 1B.

The promoter can be any promoter that is active in CD8⁺ T cells. In some embodiments, the promoter is a constitutively active promoter. The promoter can be, but is not limited to, an MNDU3 promoter, an EF-1alpha Promoter, a CMV promoter, an Igκ promoter, a mPGK, an SV40 promoter, a β-actin promoter (such as, but not limited to a human or chicken β-actin promoter), an α-actin promoter, an SRα promoter, a herpes thymidine kinase promoter, a herpes simplex virus (HSV) promoter, a mouse mammary tumor virus long terminal repeat (LTR) promoter, an adenovirus major late promoter (Ad MLP), or a rous sarcoma virus (RSV) promoter. In some embodiments, the promoter is an MNDU3 promoter. In some embodiments, the promoter is an EF-1alpha Promoter. In some embodiments, the promoter further contains a Kozak sequence.

The constructs in FIG. 1 illustrate the TCRβ chain before the TCRα chain. However, the positions of the TCRβ and TCRα chains can be switched.

2A encodes a self-cleaving peptide. Each 2A peptide is independently selected from the group consisting of a P2A, T2A, E2A and F2A. In some embodiments, the two 2A peptides are different (e.g., P2A and T2A). A linker, such as a GSG linker can be inserted before and/or after each 2A element.

The marker is optional and if present encodes a detection marker and/or a removal marker or a combination detection/removal marker. In some embodiments, the detection/removal marker comprises RQR8.

The expression cassette can have a single stop codon, a pair or stop codons, or three stop codons (STOP in FIG. 1). If three stop codons are present, the three stop codons can be configured such that a stop codon is present in each of the three forward reading frames.

The PRE comprises a post-transcriptional response element. The PRE can be, but is not limited to, a woodchuck hepatitis virus post-transcriptional regulatory element (WPRE).

In some embodiments, a plasmids for generating lentiviral vectors are described. Such plasmids contain the heterologous TCR expression cassettes as described above. The plasmids for generating lentiviral vectors further comprise one or more elements for packaging the heterologous TCR expression cassette into a lentiviral vector.

In some embodiments, such plasmids comprise an RSV-LTR/HIV LTR element located 5′ of the promoter and A3′ LTR element located 3′ of the PRE. A A3′ LTR comprises a 3′ LTR modified to form a self-inactivating vector, disrupt the promoter/enhancer activity of the LTR, and reduce potential for positional mutagenesis upon integration. The RSV-LTR/HIV LTR and 3′ALTR can facilitate integration of the expression construct into an CD8⁺ T cell.

In some embodiments, the plasmids further comprise a central polypurine tract/central termination sequence (cPPT/CTS) element located 5′ of the promoter. The cPPT/CTS can increase nuclear importation of the viral genome during T cell transduction with a viral vector and/or improve lentivirus infection of human hematopoietic primary cells.

In some embodiments, a four-plasmid transfection is used to produce self-inactivating (replication incompetent) lentivirus vectors for use in transducing T cells with a nucleic acid encoding the heterologous TCR.

Table 1 lists elements in an exemplary lentivirus plasmid and includes the location and size of each element.

TABLE 1
Identification and Location of Elements
in an exemplary Lentivirus Plasmid

| From | To | Element |
|---|---|---|
| 1 | 181 | Truncated 5′ LTR |
| 182 | 2124 | 5′ HIV arm (including RRE and cPPT) |
| 2137 | 2683 | MNDU3 Promoter |
| 2689 | 2701 | Optimized Kozak sequence |
| 2699 | 3631 | 41-10A TCR beta chain |
| 3632 | 3640 | GSG linker |
| 3641 | 3697 | P2A peptide element |
| 3698 | 4522 | 41-10A TCR alpha chain |
| 4523 | 4531 | GSG linker (codon diversified) |
| 4532 | 4585 | T2A peptide element |
| 4586 | 5059 | RQR8 cell marker/removal peptide |
| | | (CD34/CD20 epitopes) |
| 5064 | 5072 | 3-Frame Stop codons (optimized) |
| 5078 | 5666 | WPRE element |
| 5750 | 5983 | HIV 3′ LTR (ΔU3) |
| 6055 | 6176 | SV40 PolyA signal |
| 6216 | 6351 | SV40 origin of replication |
| 7108 | 7696 | pUC origin of replication |
| 7820 | 8629 | Aminoglycoside phosphotransferase |
| | | (Kanamycin resistance) [antisense] |
| 8630 | 8721 | AmpR Promoter [antisense] |
| 9020 | 9246 | RSV Promoter |
| | 9246 bp | Full plasmid size (BSBPD-104) |
| | 5983 bp | lentivirus genome size (BVP-104) |
| | 6036 bp | Predicted provirus integration (PVI-104) |

Also described are nucleic acids encoding anti-HA-1 “H” epitope TCRs (anti-HA-1 TCRs). In some embodiments, the nucleic acid comprises a sequence encoding SEQ TD NO: 20 or SEQ TD NO: 22. In some embodiments, the nucleic acid comprises a sequence having at least 75% identity to SEQ TD NO: 19, SEQ TD NO: 21, SEQ TD NO: 23, or SEQ TD NO: 24 and encoding the amino acid sequence of SEQ ID NO: 20 or SEQ TD NO: 22; or a sequence comprising SEQ TD NO: 19, SEQ ID NO: 21, SEQ ID NO: 23, or SEQ TD NO: 24. In some embodiments, the nucleic acid comprises the sequence of SEQ ID NO: 7 or SEQ TD NO: 11. In some embodiments, the nucleic acid comprises the sequence of SEQ TD NO: 13 or SEQ ID NO: 17.

Also described are nucleic acids encoding anti-HA-2 “V” epitope TCRs (anti-HA-2 TCRs). In some embodiments, a exemplary nucleic acid encoding anti-HA-2 “V” epitope TCRs comprises a nucleic acid encoding:
(a) an alpha variable region having the amino acid SEQ ID NO: 25 and a beta variable region having the amino acid sequence of SEQ ID NO: 27;
(b) an alpha variable region having the amino acid SEQ ID NO: 29 and a beta variable region having the amino acid sequence of SEQ ID NO: 31;
(c) an alpha variable region having the amino acid SEQ ID NO: 33 and a beta variable region having the amino acid sequence of SEQ ID NO: 35;
(d) an alpha variable region having the amino acid SEQ ID NO: 37 and a beta variable region having the amino acid sequence of SEQ ID NO: 39;
(e) an alpha variable region having the amino acid SEQ ID NO: 41 and a beta variable region having the amino acid sequence of SEQ ID NO: 43;
(f) an alpha variable region having the amino acid SEQ ID NO: 45 and a beta variable region having the amino acid sequence of SEQ ID NO: 47;
(g) an alpha variable region having the amino acid SEQ ID NO: 49 and a beta variable region having the amino acid sequence of SEQ ID NO: 51;
(h) an alpha variable region having the amino acid SEQ ID NO: 53 and a beta variable region having the amino acid sequence of SEQ ID NO: 55;
(i) an alpha variable region having the amino acid SEQ ID NO: 57 and a beta variable region having the amino acid sequence of SEQ ID NO: 59;
(j) an alpha variable region having the amino acid SEQ ID NO: 61 and a beta variable region having the amino acid sequence of SEQ ID NO: 63;
(k) an alpha variable region having the amino acid SEQ ID NO: 65 and a beta variable region having the amino acid sequence of SEQ ID NO: 67;
(l) an alpha variable region having the amino acid SEQ ID NO: 69 and a beta variable region having the amino acid sequence of SEQ ID NO: 71;
(m) an alpha variable region having the amino acid SEQ ID NO: 73 and a beta variable region having the amino acid sequence of SEQ ID NO: 75;
(n) an alpha variable region having the amino acid SEQ ID NO: 77 and a beta variable region having the amino acid sequence of SEQ ID NO: 79;
(o) an alpha variable region having the amino acid SEQ ID NO: 81 and a beta variable region having the amino acid sequence of SEQ ID NO: 83; or
(p) an alpha variable region having the amino acid SEQ ID NO: 85 and a beta variable region having the amino acid sequence of SEQ ID NO: 87.

In some embodiments, the nucleic acid sequences encoding the alpha and beta regions of an anti-HA-2 TCR are provided on a single vector or expression cassette and expressed from a single promoter. In some embodiments, the alpha and beta TCR chains are encoded on a single transcript and separated by a 2A element (e.g., a p2A or T2A element).

In some embodiments, a exemplary nucleic acid encoding anti-HA-2 “V” epitope TCRs comprises a nucleic acid encoding:
(a) an alpha chain having the amino acid SEQ ID NO: 26 and a beta chain having the amino acid sequence of SEQ ID NO: 28;
(b) an alpha chain having the amino acid SEQ ID NO: 30 and a beta chain having the amino acid sequence of SEQ ID NO: 32;
(c) an alpha chain having the amino acid SEQ ID NO: 34 and a beta chain having the amino acid sequence of SEQ ID NO: 36;
(d) an alpha chain having the amino acid SEQ ID NO: 38 and a beta chain having the amino acid sequence of SEQ ID NO: 40;
(e) an alpha chain having the amino acid SEQ ID NO: 42 and a beta chain having the amino acid sequence of SEQ ID NO: 44;
(f) an alpha chain having the amino acid SEQ ID NO: 46 and a beta chain having the amino acid sequence of SEQ ID NO: 48;
(g) an alpha chain having the amino acid SEQ ID NO: 50 and a beta chain having the amino acid sequence of SEQ ID NO: 52;
(h) an alpha chain having the amino acid SEQ ID NO: 54 and a beta chain having the amino acid sequence of SEQ ID NO: 56;
(i) an alpha chain having the amino acid SEQ ID NO: 58 and a beta chain having the amino acid sequence of SEQ ID NO: 60;
(j) an alpha chain having the amino acid SEQ ID NO: 62 and a beta chain having the amino acid sequence of SEQ ID NO: 64;
(k) an alpha chain having the amino acid SEQ ID NO: 66 and a beta chain having the amino acid sequence of SEQ ID NO: 68;
(l) an alpha chain having the amino acid SEQ ID NO: 70 and a beta chain having the amino acid sequence of SEQ ID NO: 72;
(m) an alpha chain having the amino acid SEQ ID NO: 74 and a beta chain having the amino acid sequence of SEQ ID NO: 76;
(n) an alpha chain having the amino acid SEQ ID NO: 78 and a beta chain having the amino acid sequence of SEQ ID NO: 80;
(o) an alpha chain having the amino acid SEQ ID NO: 82 and a beta chain having the amino acid sequence of SEQ ID NO: 84; or
(p) an alpha chain having the amino acid SEQ ID NO: 86 and a beta chain having the amino acid sequence of SEQ ID NO: 88.

In some embodiments, the nucleic acid sequences encoding the alpha and beta chains of an anti-HA-2 TCR are provided on a single vector or expression cassette and expressed from a single promoter. In some embodiments, the alpha and beta TCR chains are encoded on a single transcript and separated by a 2A element (e.g., a p2A or T2A element).

Also described are lentiviral vectors suitable for use in transducing a T cell to insert a nucleic acid encoding a heterologous anti-HA-1 TCR. In some embodiments, the lentiviral vector comprises a nucleic acid sequence encoding SEQ ID NO: 20 or SEQ ID NO: 22; a nucleic acid sequence having at least 75% identity to SEQ ID NO: 19, SEQ ID NO: 21, or SEQ ID NO: 23 and encoding the amino acid sequence of SEQ ID NO: 20 or SEQ ID NO: 22; or a nucleic acid sequence comprising SEQ ID NO: 19, SEQ ID NO: 21, or SEQ ID NO: 23.

Also described are lentiviral vectors suitable for use in transducing a T cell to insert a nucleic acid encoding a heterologous anti-HA-2 TCR. The lentiviral vector suitable for use in transducing a T cell to insert a nucleic acid encoding a heterologous anti-HA-2 TCR can comprise a nucleic acid sequence encoding any of the described Table 2.

TABLE 2
Anti-HA-2 “V” epitope TCRs (anti-HA-2 TCRs).

| | Alpha | Beta | | |
|---|---|---|---|---|
| | variable | variable | Alpha | Alpha |
| | region | region | Chain | Chain |
| Anti-HA-2 | (SEQ | (SEQ | (SEQ | (SEQ |
| TCR | ID NO) | ID NO) | ID NO) | ID NO) |
| TCR A | 25 | 27 | 26 | 28 |
| TCR B | 29 | 31 | 30 | 32 |
| TCR C | 33 | 35 | 34 | 36 |
| TCR D | 37 | 39 | 38 | 40 |
| TCR E | 41 | 43 | 42 | 44 |
| TCR F | 45 | 47 | 46 | 48 |
| TCR G | 49 | 51 | 50 | 52 |
| TCR H | 53 | 55 | 54 | 56 |
| TCR I | 57 | 59 | 58 | 60 |
| TCR J | 61 | 63 | 62 | 64 |
| TCR K | 65 | 67 | 66 | 68 |
| TCR L | 69 | 71 | 70 | 72 |
| TCR M | 73 | 75 | 74 | 76 |
| TCR N | 77 | 79 | 78 | 80 |
| TCR O | 81 | 83 | 82 | 84 |
| TCR P | 85 | 87 | 86 | 88 |

### V. Methods of Use

Described are personalized allogeneic ex vivo engineered TCR cell therapies. The engineered T cells made using the described methods can be used in the personalized allogeneic ex vivo engineered TCR cell therapy. The engineered T cells are HLA-matched, indicating they are derived from a donor that is HLA-matched to the recipient subject.

The engineered T cells can be used to provide an immune response in a recipient subject, treat cancer, induce an immune response against a cancer, kill cancer cells in a recipient subject, reduce tumor burden in a recipient subject, reduce cancer cell growth in a recipient subject, or treat an infection. The engineered T cells can be administered to a recipient subject to kill cells in the recipient subject that express the antigen recognized by the heterologous TCR or CAR. The engineered T cells kill cells that are homozygous or heterozygous for the antigen recognized by the heterologous TCR or CAR, but not cells that do not express the antigen recognized by the heterologous TCR or CAR.

The described engineered T cells are made using T cells from donor subject that is HLA-matched to the recipient subject, but does not express the antigen recognized by the heterologous TCR or CAR expressed by the engineered T cells. The recipient subject is HLA-matched to the donor subject and expresses the antigen recognized by the heterologous TCR or CAR expressed by the engineered T cells. The recipient subject can be homozygous or heterozygous for the gene that encodes/expresses the antigen. In some embodiments, the donor subject is an HLA 10/10 match to the recipient subject. In some embodiments, the donor subject is a related donor that is an HLA 10/10 match to the recipient subject. In some embodiments, the donor subject is a unrelated donor that is an HLA 10/10 match to the recipient subject. In some embodiments, the donor subject is an HLA 11/12 match to the recipient subject. In some embodiments, the donor subject is a related donor that is an HLA 11/12 match to the recipient subject. In some embodiments, the donor subject is an unrelated donor that is an HLA 11/12 match to the recipient subject. In some embodiments, the donor subject is an HLA 12/12 match to the recipient subject. In some embodiments, the donor subject is a related donor that is an HLA 12/12 match to the recipient subject. In some embodiments, the donor subject is an unrelated donor that is an HLA 11/12 match to the recipient subject.

The engineered T cells made using the described methods can be used in combination with one or more additional therapies. In some embodiments, the engineered T cells are administered to a recipient subject in combination with allogeneic hematopoietic stem cell transplant (alloSCT). In some embodiments, the engineered T cells are used in combination with CD34-selected alloSCT. The described engineered T cells can be used to increase the efficacy of alloSCT, increase the number of patients eligible for alloSCT, increase graft vs. leukemia effect in recipient subjects receiving alloSCT, and/or reduce graft vs. host disease in recipient subjects receiving alloSCT.

AlloSCT involves transferring the stem cells (graft) from a healthy person (the donor) to the patient's body, typically after a conditioning regimen. AlloSCT (also termed hematopoietic cell transplantation (HCT)) can used to treat hematopoietic disorders, including leukemia and myelodysplastic syndromes (MDS). In HLA-matched alloSCT, alloreactive donor T cells target minor histocompatibility antigens (miHAs). MiHAs are peptide products of coding polymorphisms that distinguish recipients from donors. In standard hematopoietic cell transplantation (HCT), these miHA-reactive T cells can target leukemic cells, mediating the graft-vs-leukemia (GVL) effect. However, the donor T cells can also cause graft-vs-host disease (GVHD). Mature αβ T cells contained in the donor allograft can be considered in two broad classes. One class promotes the reconstitution of anti-pathogen immunity, especially through the transfer of memory T cells. A second class of T cells, called alloreactive T cells, recognizes the patient as “non-self”. When alloSCT is used for the treatment of hematological malignancies, alloreactive donor T cells can kill malignant cells thereby mediating the GVL effect. However, they can also cause GVHD, wherein alloreactive T cells attack nonmalignant host tissues, including the skin, bowel and liver. While alloSCT can be a curative therapy for patients with hematologic malignancies, post-transplant relapse remains the greatest single cause of post-transplant death, occurring in up to 80% of high-risk patients. AlloSCT can also be used for with nonmalignant but medically serious conditions such as hemoglobinopathies, thalassemias and autoimmune diseases. AlloSCT can also be used to create tolerance to transplanted solid organs.

The described methods utilize the described engineered T cells to target malignant cells in the recipient subject. The described engineered T cells mediate GVL while reducing the risk of GVHD relative to polyclonal donor T cells.

In some embodiments, the same donor subject is used as a source of the apheresis product or PBMCs for use in manufacturing the engineered T cells and as a source for the alloSCT graft. The donor subject can undergo a single apheresis to collect the apheresis product or PBMCs and the graft or the donor subject can undergo a first apheresis to collect the apheresis product or PBMCs and a second apheresis to provide the graft. In some embodiments, the donor subject is administered a mobilizing agent to prior to apheresis. In some embodiments, the donor subject is not administered a mobilizing agent prior to the apheresis. Mobilization is a process in which certain drugs (mobilization agents) are used to cause the movement of stem cells from the bone marrow into the blood in a donor subject. A “mobilizing agent” stimulates the bone marrow to produce granulocytes and stem cells (e.g., CD34⁺ cells) and release them into the bloodstream. A mobilizing agent is administered to a subject prior to a blood donation (e.g., apheresis or leukapheresis) to increase the number of hematopoietic stem cells in the blood of the donor before collection. A mobilization agent can be, but is not limited to, Granulocyte colony stimulating factor (G-CSF), G-CSF plus a CXCR4 antagonist (e.g., plerixafor or YF-H-2015005); G-CSF plus cytarabine, or G-CSF plus cyclophosphamide.

In some embodiments, a donor subject undergoes a first apheresis to provide the apheresis product or PBMCs and a second apheresis to provide the graft, wherein the donor subject is not administered a mobilizing agent prior to the first apheresis and is administered a mobilizing agent to prior to the second apheresis. The first apheresis can be a low-volume apheresis. The second apheresis can be performed about 7 to about 14 days after the first apheresis. In some embodiments, the second apheresis is a G-CSF-mobilized apheresis.

In some embodiments, the alloSCT graft is a CD34-selected alloSCT graft. CD34⁺ selection removes donor T cells from the peripheral blood graft. CD34-selected indicates that the graft is enriched in CD34⁺ cells. In addition to reducing GVHD, CD34⁺ selection of the graft provides for administration of the engineered T cells at a nadir of the recipient lymphocyte population. By administering the engineered T calls in combination with a CD34-selected graft and administering the engineered T cells on the same day as the CD34-selected graft, the engineered T cells are able to grow and proliferate in the absence of competition from donor or recipient T cells or leukemia cells.

Positive CD34⁺ selection can deplete T cell from the graft CD34 selection can be performed using methods available in the art. Such methods include, but are not limited to, ISOLEX 300i, the CliniMA CS CD34 Reagent System, and αβ+ TCR/CD19 depletion CD34⁺ selection can result in up to a 4-5 log reduction of T cells in the final graft. In some embodiments, the graft contains fewer than 50,000, fewer than 25,000, fewer than 10,000 or fewer than 5,000 T cells/kg for a graft dose of 5×10⁶ cells/kg. In some embodiments, the graft contains less than 1% T cells. CD34⁺ selection is performed prior to administration of the graft to the recipient subject.

In some embodiments, the alloSCT graft and engineered T cells are administered to the recipient subject in the absence of prophylactic immunosuppression therapy targeting graft vs. host disease (GVHD; e.g., acute GVHD) or immune activity of T cells. In other words, the alloSCT graft and the engineered T cells are intended to be administered to the recipient subject without also administering any immunosuppression therapy that targets GVHD or suppresses immune activity of T cells prior to observation of GVHC or any symptoms of (GVHD in the recipient subject. In some embodiments, no immunosuppressive therapy targeting GVHD is prophylactically administered to the recipient subject prior to, concomitantly with, or subsequent to administration of the alloSCT. In some embodiments, no immunosuppressive therapy targeting GVHD is prophylactically administered to the recipient subject prior to administration of the alloSCT. In some embodiments, no immunosuppressive therapy targeting GVHD is prophylactically administered to the recipient subject concomitantly with administration of the alloSCT. In some embodiments, no immunosuppressive therapy targeting GVHD is prophylactically administered to the recipient subject subsequent to administration of the alloSCT. In some embodiments, no immunosuppressive therapy targeting GVHD is prophylactically administered to the recipient subject prior to or concomitantly with administration of the alloSCT. In some embodiments, no immunosuppressive therapy targeting GVHD is prophylactically administered to the recipient subject prior to or subsequent to administration of the alloSCT. In some embodiments, no immunosuppressive therapy targeting GVHD is prophylactically administered to the recipient subject concomitantly with or subsequent to administration of the alloSCT. In some embodiments, no immunosuppressive therapy targeting GVHD is prophylactically administered to the recipient subject from about 1 week prior to administration of the alloSCT to at least 1 week after administration of the alloSCT. In some embodiments, no immunosuppressive therapy targeting GVHD is prophylactically administered to the recipient subject from about 1 week prior to administration of the alloSCT to at least 2 weeks after administration of the alloSCT. In some embodiments, no immunosuppressive therapy targeting GVHD is prophylactically administered to the recipient subject from about 1 week prior to administration of the alloSCT to about 4 weeks after administration of the alloSCT. In some embodiments, no immunosuppressive therapy targeting GVHD is prophylactically administered to the recipient subject from about 1 week prior to administration of the alloSCT to about 3 weeks after administration of the alloSCT. In some embodiments, no immunosuppressive therapy targeting GVHD is prophylactically administered to the recipient subject from about 1 week prior to administration of the alloSCT to about 2 weeks after administration of the alloSCT. In some embodiments, no immunosuppressive therapy targeting GVHD is prophylactically administered to the recipient subject for at least 1 week after following administration of the alloSCT. In some embodiments, no immunosuppressive therapy targeting GVHD is prophylactically administered to the recipient subject for at least 2 weeks after following administration of the alloSCT. In some embodiments, no immunosuppressive therapy targeting GVHD is prophylactically administered to the recipient subject for at least 3 weeks after following administration of the alloSCT In some embodiments, no immunosuppressive therapy targeting GVHD is prophylactically administered to the recipient subject for at least 4 weeks after following administration of the alloSCT. Immunosuppressive therapy targeting GVHD can be administered to the recipient subject following administration of the alloSCT if the recipient subject exhibits one or more symptoms of GVHD. Prophylactic immunosuppression is typically administered to a recipient subject receiving alloSCT. However, by selecting a donor having a 10/10 (related) or 11-12/12 (unrelated) HLA match to the recipient, selecting a donor that does not contain a miHA antigen that is present in the recipient, and administering a CD34-selected graft and engineered T cells expressing a TCR specific for the miHA antigen of the recipient together on day 1, the need for prophylactic immunosuppression is reduced or eliminated. Immunosuppressive agents that target GVHD or suppresses T cells activation or proliferation include, but are not limited to, abatacept, antithymocyte globulin (ATG), alemtuzumab, T cell-depleting antibody preparations, corticosteroids (e.g., methylprednisolone, prednisone. dexamethasone, beclomethasone, or budesonide), cyclophosphamide, cyclosporine, methotrexate, mycophenolate mofetil, sirolimus, tacrolimus, calcineurin-inhibitors, kinase-inhibitors, antiproliferative drugs such as mycophenolate or alkylating agents, and anti-cytokine biologics.

One or more drugs that suppress cytokine release syndrome can be administered to the recipient subject. Drugs that suppress cytokine release syndrome can be administered to the recipient subject as needed or prophylactically. Drugs that suppress cytokine release syndrome include, but are not limited to, anti-IL-6 therapies, tocilizumab, sarilumab, anakinra, siltuximab, and corticosteroids.

Described are methods of treating a recipient subject suffering from a hematological malignancy comprising: administering an effective dose of engineered T cells to the recipient subject within five days of administering an allogeneic stem cell transplant (alloSCT), wherein no immunosuppressive agent that targets GVHD is prophylactically administered to the recipient subject to suppress the immune activity of T cells. The alloSCT can administered using a CD34-selected alloSCT graft. In some embodiments, the effective dose of engineered T cells is administered to the recipient subject within four days of the alloSCT In some embodiments, the effective dose of engineered T cells is administered to the recipient subject within three days of the alloSCT. In some embodiments, the effective dose of engineered T cells is administered to the recipient subject within two days of the alloSCT. In some embodiments, the effective dose of engineered T cells is administered to the recipient subject within 72 hours of the alloSCT. In some embodiments, the engineered T cells are administered to the recipient subject within 48 hours of administering the CD34-selected alloSCT graft to the recipient subject. In some embodiments, the engineered T cells are administered to the recipient subject within 24 hours of administering the CD34-selected alloSCT graft to the recipient subject. In some embodiments, the engineered T cells are administered to the recipient subject within 18 hours of administering the CD34-selected alloSCT graft to the recipient subject. In some embodiments, the engineered T cells are administered to the recipient subject within 12 hours of administering the CD34-selected alloSCT graft to the recipient subject. In some embodiments, the engineered T cells are administered to the recipient subject within 6 hours of administering the CD34-selected alloSCT graft to the recipient subject. Administration of the described engineered T cells to a recipient subject receiving alloSCT reduces or eliminates the need to administer prophylactic immunosuppressive therapy to the recipient subject to prevent GVHD.

In some embodiments, the engineered T cells are administered to the recipient subject within one day (e.g., within 24 hours) of the alloSCT. In some embodiments, the engineered T cells are administered to the recipient subject on the same day as the alloSCT. In some embodiments, the engineered T cells arc administered to the recipient subject following, and on the same day, as the alloSCT. Without wishing to be bound by theory, administering the engineered T cells at the same time and the graft allows the engineered T cells to grow and expand in the depleted immune system of the recipient subject (e.g., in the absence of significant leukemia cell growth or T cell proliferation from the graft), thus allowing the engineered T cells to integrate with developing immune system. In contrast, when administering the engineered T cells to a recipient subject 2-4 weeks after the alloSCT, as is typically done, the engineered T cells must compete with proliferating residual host cells, proliferating leukemia cells, and/or proliferating alloSCT cells.

In some embodiments, the recipient subject receives a conditioning regimen prior to the alloSCT. The conditioning regimen comprises chemotherapy that kills the recipient subject's immune cells (including leukemia cells) and creates space in the recipient's bone marrow for donor stem cell engraftment. A conditioning regimen is a pre-transplant treatment that provides immunoablation to prevent graft rejection and reduce tumor burden. The conditioning regimen can be a high-dose (myeloablative) regimen, a reduced intensity regimen, or a nonmyeloablative regiment. A high-dose regimen, consisting of alkylating agents (single or multiple) with or without total body irradiation, is expected to ablate marrow hematopoiesis. A high-dose regimen is not expected to allow autologous hematologic recovery. Exemplary high-dose regimens include, but are not limited to, TBI plus cyclophosphamide, busulfan plus cyclophosphamide, and busulfan plus melphalan). Reduced-intensity regimens are those that do not fit the definition for myeloablative or nonmyeloablative conditioning. Reduced-intensity regimens result in potentially prolonged cytopenias and require hematopoietic stem cell support. The dose of alkylating agents or total body irradiation in reduced-intensity regimen is generally reduced by ≥30% compared to high-dose regimens. Exemplary reduced-intensity regimens include, but are not limited to, melphalan plus fludarabine or melphalan plus cladribine. Nonmyeloablative conditioning is not expected to not require stem cell support. Conditioning regiments includes, but are not limited to, (a) TBI/Fludarabine/Thiotepa (TBI 1375 cGy, hyperfractionated; Fludarabine 25 mg/m²/day over 5 days (total 125 mg/m²); Thiotepa—5 mg/kg dosed over 2 days (total 10 mg/kg)) and (b) Busulfan/Melphalan/Fludarabine: (Busulfan (>7.2 mg/kg IV); Melphalan, 140 mg/m², over 2 days; Fludarabine 25 mg/m²/day over 5 days (total 125 mg/m²)).

Described are methods of treating a recipient subject suffering from a hematological malignancy comprising: administering an alloSCT and administering an effective dose of engineered T cells to the recipient subject, wherein the engineered T cells express a heterologous TCR that recognizes a hematopoietic-restricted miHA antigen expressed by the recipient subject, wherein the engineered T cells are administered to the recipient subject on the same day (e.g., within 24 hours) as the alloSCT, and wherein no immunosuppressive agent that targets GVHD is prophylactically administered to the recipient subject to suppress the immune activity of T cells. The hematological malignancy can be, but it not limited to, acute myeloid leukemia (AML), myelodysplastic syndrome (MDS), or acute lymphoblastic leukemia (ALL). In some embodiments, the recipient subject has measurable disease or one or more risk factors or indicators of poor outcome prior to the alloSCT. The alloSCT can administered using a CD34-selected alloSCT graft. In some embodiments, the engineered T cells are derived from a related or unrelated donor that is HLA 10/10 matched to the recipient subject or a related or unrelated donor that is HLA 11/12 or 12/12 matched to the recipient subject, wherein the donor does not express the hematopoietic-restricted miHA antigen recognized by the heterologous TCR. In some embodiments, the T cells used in generating the engineered T cells and the alloSCT graft are obtained from the same donor.

Described are methods of treating a recipient subject suffering from a hematological malignancy comprising: administering a conditioning regimen to the recipient subject, administering an alloSCT, and administering an effective dose of engineered T cells to a recipient subject, wherein the engineered T cells express a heterologous TCR that recognizes a hematopoietic-restricted miHA antigen expressed by the recipient subject, wherein the engineered T cells are administered to the recipient subject on the same day (e.g., within 24 hours) as the allSCT and wherein no immunosuppressive agent that targets GVHD is prophylactically administered to the recipient subject to suppress the immune activity of T cells. The hematological malignancy can be, but it not limited to, AML, MDS, or ALL. In some embodiments, the recipient subject has measurable disease or one or more risk factors or indicators of poor outcome prior to the alloSCT. The conditioning regimen can be a high-dose (myeloablative) regimen, a reduced intensity regimen, or a nonmyeloablative regiment. The recipient subject is not prophylactically treated with any immunosuppressive agent that targets GVHD or suppresses the immune activity of T cells. The alloSCT can administered using a CD34-selected alloSCT graft. In some embodiments, the engineered T cells are derived from a related or unrelated donor that is HLA 10/10 matched to the recipient subject or a related or unrelated donor that is HLA 11/12 or 12/12 matched to the recipient subject, wherein the donor does not express the hematopoietic-restricted miHA antigen recognized by the heterologous TCR. In some embodiments, the T cells used in generating the engineered T cells and the alloSCT graft are obtained from the same donor.

Described are methods of treating a recipient subject suffering from a hematological malignancy and having measurable residual disease following at least one prior therapy comprising: administering a conditioning regimen to the recipient subject, administering an alloSCT, and administering an effective dose of engineered T cells, wherein the engineered T cells express a heterologous TCR the recognizes a hematopoietic-restricted miHA antigen expressed by the recipient subject, wherein the engineered T cells are administered to the recipient subject on the same day (e.g., within 24 hours) as the allSCT and wherein no immunosuppressive agent that targets GVHD is prophylactically administered to the recipient subject to suppress the immune activity of T cells. The hematological malignancy can be, but it not limited to, AML, MDS, or ALL. In some embodiments, the recipient subject has one or more risk factors or indicators of poor outcome prior to the alloSCT. The conditioning regimen can be a high-dose (myeloablative) regimen, a reduced intensity regimen, or a nonmyeloablative regiment. The alloSCT can administered using a CD34-selected alloSCT graft. The recipient subject is not prophylactically treated with any immunosuppressive agent that targets GVHD or suppresses the immune activity of T cells. In some embodiments, the engineered T cells are derived from a related or unrelated donor that is HLA 10/10 matched to the recipient subject or a related or unrelated donor that is HLA 11/12 or 12/12 matched to the recipient subject, wherein the donor does not express the hematopoietic-restricted miHA antigen recognized by the heterologous TCR In some embodiments, the T cells used in generating the engineered T cells and the alloSCT graft are obtained from the same donor.

Described are methods of treating a recipient subject suffering from a hematological malignancy and one or more risk factors or indicators of poor outcome comprising: administering a conditioning regimen to the recipient subject, administering an alloSCT, and administering an effective dose of engineered T cells, wherein the engineered T cells express a heterologous TCR the recognizes a hematopoietic-restricted miHA antigen expressed by the recipient subject, wherein the engineered T cells are administered to the recipient subject on the sane day as the alloSCT and wherein no immunosuppressive agent that targets GVHD is prophylactically administered to the recipient subject to suppress the immune activity of T cells. The hematological malignancy can be, but it not limited to, AML, MDS, or ALL. The conditioning regimen can be a high-dose (myeloablative) regimen, a reduced intensity regimen, or a nonmyeloablative regiment. The alloSCT can administered using a CD34-selected alloSCT graft. The recipient subject is not prophylactically treated with any immunosuppressive agent that targets GVHD or suppresses the immune activity of T cells. In some embodiments, the engineered T cells are derived from a related donor that is HLA 10/10 matched to the recipient subject or a related or unrelated donor that is HLA 11/12 or 12/12 matched to the recipient subject, wherein the donor does not express the hematopoietic-restricted miHA antigen recognized by the heterologous TCR. In some embodiments, the T cells used in generating the engineered T cells and the alloSCT graft are obtained from the same donor.

Described are methods of treating a recipient subject suffering from a hematological malignancy and having measurable residual disease following at least one prior therapy and/or one or more risk factors or indicators of poor outcome comprising:
(a) selecting an HLA-matched donor and performing an apheresis procedure to collect an apheresis product from the donor,
(b) genetically modifying CD8⁺ T cells from the apheresis product to knock out the endogenous TRAC and TRBC genes and express a heterologous TCR that recognizes a hematopoietic-restricted miHA antigen expressed by the recipient subject but not by the donor thereby generating engineered T cells;
(c) administering a mobilizing agent to the donor and performing a second apheresis procedure to collect a second apheresis product from the donor;
(d) selecting CD34 cells from the apheresis product to from a CD34-selected alloSCT graft;
(d) administering a conditioning regimen to the recipient subject; and
(e) administering the CD34-selected alloSCT graft and an effective dose of the engineered T cells to the recipient subject, wherein the engineered T cells are administered to the recipient subject within 72 hours of administering the CD34-selected alloSCT graft to the recipient subject;
wherein no immunosuppressive agent that targets GVHD is prophylactically administered to the recipient subject to suppress the immune activity of T cells.

In some embodiments, PBMCs are isolated from the apheresis product. The hematological malignancy can be, but it not limited to, AML, MDS, or ALL. The conditioning regimen can be a high-dose (myeloablative) regimen, a reduced intensity regimen, or a nonmyeloablative regiment. The donor can be a related or unrelated donor that is HLA 10/10 matched to the recipient subject or a related or unrelated donor that is HLA 11/12 or 12/12 matched to the recipient subject. In some embodiments, the engineered T cells are administered to the recipient subject within 48 hours of administering the CD34-selected alloSCT graft to the recipient subject. In some embodiments, the engineered T cells are administered to the recipient subject within 24 hours of administering the CD34-selected alloSCT graft to the recipient subject. In some embodiments, the engineered T cells are administered to the recipient subject within 18 hours of administering the CD34-selected alloSCT graft to the recipient subject. In some embodiments, the engineered T cells are administered to the recipient subject within 12 hours of administering the CD34-selected alloSCT graft to the recipient subject. In some embodiments, the engineered T cells are administered to the recipient subject within 6 hours of administering the CD34-selected alloSCT graft to the recipient subject.

Described are methods of treating a recipient subject suffering from a hematological malignancy and having measurable residual disease following at least one prior therapy and/or having one or more risk factors or indicators of poor outcome comprising:
(a) generating engineered T cells by genetically modifying CD8⁺ T cells from a donor apheresis product to knock out the endogenous TRAC and TRBC genes and express a heterologous TCR that recognizes a hematopoietic-restricted miHA antigen expressed by the recipient subject, wherein the donor is HLA-matched to the recipient subject but does not express the hematopoietic-restricted miHA antigen; and
(b) administering an effective dose of the engineered T cells to the recipient subject in combination with a CD34-selected alloSCT graft, wherein the CD34-selected alloSCT graft is from the HLA-matched donor and wherein the effective dose of the engineered T cells is administered to the recipient subject within 72 hours, within 48 hours, within 24 hours, within 18 hours, within 12 hours, or within 6 hours of the CD34-selected alloSCT graft, wherein no immunosuppressive agent that targets GVHD is prophylactically administered to the recipient subject to suppress the immune activity of T cells.

The hematological malignancy can be, but it not limited to, AML, MDS, or ALL. The recipient subject can receive a conditioning regimen prior to administration of the CD34-selected alloSCT graft. The conditioning regimen can be a high-dose (myeloablative) regimen, a reduced intensity regimen, or a nonmyeloablative regiment. The donor can be a related or unrelated donor that is HLA 10/10 matched to the recipient subject or a related or unrelated donor that is HLA 11/12 or 12/12 matched to the recipient subject. The donor may undergo two apheresis procedures, wherein the donor is not administered a mobilizing agent prior to first apheresis and the donor is administered a mobilizing agent prior to the second apheresis. The first apheresis procedure is used to obtain a first apheresis product that is used in manufacturing the engineered T cells. The second apheresis procedure is used to obtain a second apheresis product that is used in generating the CD34-selected alloSCT graft.

The alloSCT can comprise about 2×10⁶ cells/kg to about 8×10⁶ cells/kg. In some embodiments, the alloSCT comprise about 2×10⁶ CD34⁺ cells/kg to about 8×10⁶ CD34⁺ cells/kg. The engineered T cell dose can be about 0.1×10⁶ cells/kg to 3×10⁶ cells/kg. In some embodiment, the engineered T cell dose is about 0.1×10⁶ cells/kg. In some embodiment, the engineered T cell dose is about 0.3×10⁶ cells/kg. In some embodiment, the engineered T cell dose is about 1×10⁶ cells/kg. In some embodiment, the engineered T cell dose is about 3×10⁶ cells/kg.

In some embodiments, the engineered T cells express a heterologous TCR that recognizes a miHA antigen. In some embodiments, the engineered T cells express a heterologous TCR that recognizes a miHA antigen whose express is relatively restricted to hematopoietic cells. The miHa antigen can be, but is not limited to, a miHA HA-1 antigen or a miHA HA-2 antigen. The miHA HA-1 antigen can be a miHA HA-1 “H” antigen or a miHA HA-1 “R” antigen. In some embodiments, the miHA-HA-1 antigen comprises a miHA HA-1 “H” antigen (VLHDDLLEA, SEQ ID NO: 3). In some embodiments, the miHA-HA-1 antigen comprises a miHA HA-1 “R” antigen (VLRDDLLEA, SEQ ID NO: 4). In some embodiments, the heterologous anti-miHA HA-1 TCR recognizes the miHA HA-1 antigen in the context of an MHC molecule. In some embodiments, the MHC molecule is a human leukocyte antigens (HLA)-A molecule. In some embodiments, the heterologous anti-miHA HA-1 TCR recognizes the miHA HA-1 antigen in the context an HLA-A molecule of serotype HLA-A*02:01 or HLA-A*02:06. The miHA HA-2 antigen can be a miHA HA-2 “V” antigen or a miHA HA-2 “M” antigen. In some embodiments, the miHA HA-2 antigen comprises a miHA HA-2 “V” antigen (YIGEVLVSV, SEQ ID NO: 5). In some embodiments, the miHA HA-2 antigen comprises a miHA HA-2 “M” antigen (YIGEVLVSM, SEQ ID NO: 6). In some embodiments, the heterologous anti-miHA HA-2 TCR recognizes the miHA HA-2 antigen in the context of an MHC molecule. In some embodiments, the MHC molecule is a human leukocyte antigens (HLA)-A molecule. In some embodiments, the heterologous anti-miHA HA-2 TCR recognizes the miHA HA-2 antigen in the context an HLA-A molecule of serotype HLA-A*02:01.

The described methods provide for more effective alloreactive T cell response that mediate GVL response while decreasing GVHD relative to polyclonal donor T cells. Administration of the described engineered T cells to a recipient subject in conjunction with (e.g., same day as) alloSCT can be used to lower the rate of relapse of a hematological malignancy, reduce the severity of relapse of a hematological malignancy, delay recurrence of a hematological malignancy, increase relapse-free survival, reduce toxicity associated with alloSCT, reduce treatment-related mortality of alloSCT, reduce or eliminate the need for immunosuppression therapy in alloSCT, reduce the post-transplant morbidity caused by systemic immunosuppression, promote engraftment of alloSCT, reduce immunologic rejection of the alloSCT, reduce GVHD following alloSCT, and/or reduce the risk of GVHD following alloSCT relative to the recipient subject receiving alloSCT in the absence of the engineered T cells.

The described methods can be used to treat recipient subjects that would typically not be eligible for alloSCT therapy. Such subjects include, but are not limited to, subject with measurable disease following a least one prior line of therapy (e.g., induction therapy), subjects having active disease, and subjects having one or more risk factors or indicators of poor prognosis following alloSCT.

### VI. Recipient Subject

The recipient subject (e.g., patient) is positive for an antigen recognized by a heterologous TCR or CAR expressed by the engineered T cells.

Recipient subjects amenable to treatment with the described engineered T cells and methods of using the engineered T cells include, but are not limited to, recipient subjects having a hematologic malignancy, a hemoglobinopathy, a thalassemia, a solid organ transplant, or an autoimmune disease. A hematologic malignancy can be, but is not limited to, acute lymphoblastic leukemia (ALL), acute myeloid leukemia (AML), myelodysplastic syndrome (MDS), chronic lymphocytic leukemia, chronic myeloid leukemia in blast crisis, chronic myeloid leukemia in accelerated phase, multiple myeloma, or non-Hodgkin's lymphoma. An autoimmune disease can be, but is not limited to, multiple sclerosis, myasthenia gravis, systemic sclerosis, systemic lupus erythematosus, polymyositis-dermatomyositis, Sjogren's disease, rheumatoid arthritis, juvenile chronic arthritis, psoriatic arthritis, vasculitis, immune thrombocytopenia, autoimmune hemolytic anemia, Evans syndrome, pure red/white cell aplasia, Crohn's disease, ulcerative colitis, refractory type II coeliac disease, granulomatosis with polyangiitis, Behcet's disease, chronic inflammatory demyelinating polyneuropathy, neuromyelitis optica, or inflammatory bowel disease.

In some embodiments, the recipient subject has a hematologic malignancy.

In some embodiments, the recipient subject has no measurable residual disease (MRD). In some embodiments, the recipient subject has no MRD following induction therapy. In some embodiments, the recipient subject has no MRD following consolidation therapy. The disease can be, but it not limited to, a hematologic malignancy. Measurable disease or measurable residual may be determined by morphology, flow cytometry, or molecular testing or evaluation of a bone marrow aspirate or biopsy. In some embodiments, the recipient subject has morphologically identifiable disease (e.g., leukemia). Morphologically identifiable disease indicates that cancerous cells can be detected by microscopic analysis or standard tests that examine cell samples with a microscope.

In some embodiments, the recipient subject suffers from a hematological malignancy and is refractory to at least one prior therapy. In some embodiments, the recipient subject is refractory to at least two prior therapies.

In some embodiments, the recipient subject suffers from a hematological malignancy and is at risk of relapse following induction therapy. In some embodiments, the recipient subject is at risk of relapse following consolidation therapy. In some embodiments, the recipient subject is at risk of relapse following alloSCT.

In some embodiments, the recipient subject suffers from a hematological malignancy and has not responded to induction therapy. Induction therapy (also termed remission induction therapy) is a first-line treatment of cancer with a chemotherapeutic drug. For treatment of leukemia and other hematopoietic disorders, induction therapy comprises a typically short (about 1 week), intensive therapy having the goal of clearing the blood of leukemia cells (myeloblasts; also termed blasts) and reducing the number of blasts in the bone marrow to normal (e.g., <5%). Induction therapy includes any induction therapy typically used in the art to treat a hematopoietic disorder. Induction therapies include, but are not limited to, cytarabine (cytosine arabinoside or ara-C) plus an anthracycline drug, (e.g., daunorubicin (daunomycin) or idarubicin), cladribine (2-CdA), fludarabine, mitoxantrone, etoposide (VP-16), 6-thioguanine (6-TG), hydroxyurea, corticosteroid drugs (e.g., prednisone or dexamethasone), methotrexate (MTX), 6-mercaptopurine (6-MP), azacytidine, decitabine, or clofarabine.

In some embodiments, the recipient subject suffers from a hematological malignancy and has not responded consolidation therapy. Consolidation therapy comprises a chemotherapy administered to a recipient subject after the recipient subject has recovered from induction therapy. Consolidation therapy is meant to kill the leukemia cells remaining after induction therapy.

In some embodiments, the recipient subject has no MRD, but has one or more risk factors or indicators of poor outcome. In some embodiments, the recipient subject has no MRD following induction therapy, but has one or more risk factors or indicators of poor outcome. In some embodiments, the recipient subject has no MRD following consolidation therapy, but has one or more risk factors or indicators of poor outcome. The disease can be, but it not limited to, a hematologic malignancy. In some embodiments, the recipient subject has <5% blasts in their bone marrow, but has one or more risk factors or indicators of poor outcome.

In some embodiments, the recipient subject is MRD-positive as determined by multiparameter flow cytometry that identifies myeloblasts with an abnormal immunophenotype.

In some embodiments, the recipient subject has not responded to induction therapy. In some embodiments, the recipient subject has ≥5% blasts in their bone marrow. In some embodiments, the recipient subject has ≥5% blasts in their bone marrow following induction therapy. In some embodiments, the recipient subject has ≤25% blasts in their bone marrow. In some embodiments, the recipient subject has ≤25% blasts in their bone marrow following induction therapy. In some embodiments, the recipient subject has about 5% to about 25% blasts in their bone marrow. In some embodiments, the recipient subject has about 5% to about 25% blasts in their bone marrow following induction therapy. In some embodiments, the recipient subject has detectable blasts in their peripheral blood. In some embodiments, the recipient subject has detectable blasts in their peripheral blood following induction therapy.

In some embodiments, the recipient subject is not in remission, has active disease, has MRD, has relapsed, or has circulating myeloblasts (blasts; abnormal immature white blood cells). In some embodiments, the recipient subject has active disease, has MRD, has relapsed, or has circulating myeloblasts (blasts) and one or more risk factors or indicators of poor outcome. Recipient subjects suitable for administration of the described engineered T cell therapies have ≤10% blasts, ≤15% blasts, ≤20% blasts, ≤25% blasts, or ≤30% blasts in their bone marrow. In some embodiments, the recipient subject has ≤25% blasts. In some embodiments, the recipient subject has ≥5% blasts, ≥10% blasts, or ≥15% blasts in bone marrow. In some embodiments, the recipient subject has 0% to about 25% blasts in bone marrow. In some embodiments, the recipient subject has about 5% to about 25% blasts in bone marrow. In some embodiments, the recipient subject has about 10% to about 25% blasts in bone marrow. In some embodiments, the recipient subject has about 15% to about 25% blasts in bone marrow. In some embodiments, the recipient subject has about 5% to about 20% blasts in bone marrow. In some embodiments, the recipient subject has about 5% to about 15% blasts in bone marrow. In some embodiments, the recipient subject has about 5% to about 10% blasts in bone marrow. In some embodiments, the recipient subject has detectable blasts in peripheral blood.

In some embodiments, the recipient subject is not in complete molecular remission. Complete molecular remission indicated there is no evidence of leukemia cells in the bone marrow, when using sensitive lab tests, such as polymerase chain reaction (PCR) or flow cytometry.

In some embodiments, the recipient subject has one or more risk factors or indicators of poor outcome prior to receiving alloSCT therapy. In some embodiments, the recipient subject has one or more risk factors or indicators of poor outcome following induction therapy. In some embodiments, the recipient subject has one or more risk factors or indicators of poor outcome following consolidation therapy. In some embodiments, the recipient subject has one or more risk factors or indicators of poor outcome following a prior SCT therapy. Risk factors or indicators of poor prognosis include, but are not limited to, a TP53 mutation, complex karyotype, typical complex karyotype, atypical complex karyotype, monosomal karyotype, a 17p chromosomal abnormality, or a Ph+ chromosomal abnormality. A complex karyotype indicates the recipient subject has ≥3 chromosomal abnormalities. The chromosomal abnormalities can include one or more of 5q, 7q, and 17p abnormalities. A monosomal karyotype indicates the recipient subject has two autosomal monosomies (AM) (loss of a single chromosome of a pair) or a single AM and one structural chromosomal abnormality. A Ph+ chromosomal abnormality indicates the recipient subject has a translocation between chromosomes 9 and 22 creating a BCR-ABL1 fusion gene.

In some embodiments, the recipient subject has advanced leukemia or lymphoma and/or is predicted to have a poor outcome post-transplant, and would not otherwise be eligible for alloSCT.

In some embodiment, the recipient subject is in condition for hematopoietic stem cell transplant. In some embodiment, the recipient subject is in condition for hematopoietic stem cell transplant after undergoing a conditioning regimen.

In some embodiments, the recipient subject is an AML, ALL, or MDS patient. In some embodiments, the recipient subject is an AML, ALL, or MDS patient that is refractory to one or more prior lines of therapy. In some embodiments, the recipient subject is an AML, ALL, or MDS patient that has one or more risk factors or indicators of poor outcome

In some embodiments, the recipient subject is an AML patient having measurable residual disease or measurable residual disease with up to 25% myeloblasts in bone marrow. In some embodiments, the recipient subject is an AML patient having one or more risk factors or indicators of poor prognosis. The risk factors or indicators of poor prognosis include, but are not limited to, a TP53 mutation, a complex karyotype, a monosomal karyotype, a 17p chromosomal abnormality, or a Ph+ chromosomal abnormality. An AML patient having one or more risk factors or indicators of poor prognosis can have MRD or no MRD following induction therapy. In some embodiments, the recipient subject is an AML patient having MRD-positive as determined by multiparameter flow cytometry that identifies myeloblasts with an abnormal immunophenotype. In some embodiments, the recipient subject is an AML patient having a persistent disease-defining cytogenetic abnormality or detectable core-binding factor transcripts (RUNX1-RUNXIT1 or CBFB-MYH11) or NPM1 mutant transcripts as measured by qPCR or dPCR in blood or bone marrow or any evidence of FLT3-ITD in blood or bone marrow. In some embodiments, the recipient subject is an AML patient that is MRD negative, but has a high-risk disease such as having had a TP53 mutation, complex karyotype, monosomal karyotype, abn(17p) or MECOM (EVI1) rearrangements. In some embodiments, the recipient subject is an AML patient that has failed to respond to at least one previous therapy. In some embodiments, the recipient subject is an AML patient that has failed to respond to two previous therapies.

In some embodiments, the recipient subject is an ALL patient having measurable residual disease or measurable residual disease with up to 25% myeloblasts in bone marrow. In some embodiments, the recipient subject is an ALL patient having measurable residual disease or measurable residual disease with up to 25% myeloblasts in bone marrow as determined by morphology or multiparameter flow cytometry. In some embodiments, the recipient subject is an ALL patient having one or more risk factors or indicators of poor prognosis. The risk factors or indicators of poor prognosis include, but are not limited to, Ph+ chromosomal abnormality. An ALL patient having one or more risk factors or indicators of poor prognosis can have MRD or no MRD following induction therapy. In some embodiments, the recipient subject is an ALL patient that has failed to respond induction therapy. In some embodiments, the recipient subject is an ALL patient that has failed to respond to induction therapy and consolidation therapy. In some embodiments, the recipient subject is an ALL patient that has failed to respond to induction therapy and has (a) any persistent disease-defining cytogenetic abnormality, (b) MRD positive as determined by multiparameter flow cytometry on bone marrow or peripheral blood, (c) Ph-like ALL with or without MRD, or (d) CRLF2 mutations, Ikaros deletions, monosomy 7, or complex karyotype.

In some embodiments, the recipient subject is an MDS patient having measurable residual disease or measurable residual disease with up to 25% myeloblasts in bone marrow. In some embodiments, the recipient subject is an MDS patient having a high or very high risk as determined by the Revised International Prognostic Scoring System (IPSS-R). In some embodiments, the recipient subject is an MDS patient having one or more risk factors or indicators of poor prognosis. The risk factors or indicators of poor prognosis include, but are not limited to, a complex karyotype, a monosomal karyotype, a TP53 mutation, or a mutation in RAS-pathway, JAK2, RUNX1, or ASXL1. An MDS patient having one or more risk factors or indicators of poor prognosis can have MRD or no MRD following induction therapy.

TABLE 3A
anti-HA-1 “H” epitope TCR (anti-HA-1 TCR) nucleic acid and amino acid
sequences.

SEQ ID NO: 7 anti-HA-1 “H” epitope TCR beta variable domain
ATGGGCACAAGCCTGCTGTGTTGGATGGCCCTGTGTCTGCTGGGAGCCGATCATGCCGATACGGGAGTGTCCCAGGATCCTC
GGCACAAGATTACCAAGCGGGGCCAGAACGTGACCTTCAGATGCGACCCTATCAGCGAGCACAACCGGCTGTACTGGTACAG
ACAGACACTCGGCCAGGGACCTGAGTTCCTGACCTACTTCCAGAACGAGGCCCAACTCGAAAAGAGTAGACTGCTGAGCGAC
AGATTCAGCGCCGAAAGACCCAAGGGCAGCTTCAGCACCCTGGAAATCCAGAGAACCGAGCAGGGCGACAGCGCCATGTATC
TGTGTGCCAGCTCTCTGGTGTCCGGCAACGAGCAGTTTTTCGGCCCTGGCACCAGACTGACCGTGCTGGAA
SEQ ID NO: 8 anti-HA-1 “H” epitope TCR beta variable domain
MGTSLLCWMALCLLGADHADTGVSQDPRHKITKRGQNVTFRCDPISEHNRLYWYRQTLGQGPEFLTYFQNEAQLEKSRLLSD
RFSAERPKGSFSTLEIQRTEQGDSAMYLCASSLVSGNEQFFGPGTRLTVLE
SEQ ID NO: 9 anti-HA-1 “H” epitope TCR beta constant domain
GATCTGAAGAACGTGTTCCCACCTGAGGTGGCCGTGTTCGAGCCTTCTGAGGCCGAGATCAGCCACACACAGAAAGCCACAC
TCGTGTGTCTGGCCACCGGCTTCTATCCCGATCACGTGGAACTGTCTTGGTGGGTCAACGGCAAAGAGGTGCACAGCGGCGT
CAGTACTGATCCTCAGCCTCTCAAAGAACAGCCCGCTCTGAACGACAGCCGGTACTGTCTGAGCAGCAGACTGAGAGTGTCC
GCCACCTTCTGGCAGAATCCTAGAAATCATTTCAGGTGCCAGGTGCAATTCTATGGCCTGAGCGAAAACGATGAATGGACCC
AGGATAGAGCCAAGCCTGTGACACAAATTGTGAGCGCTGAGGCATGGGGCAGAGCCGATTGTGGCTTTACCAGCGAGAGCTA
CCAGCAGGGCGTGCTGTCTGCCACAATCCTGTACGAGATCCTGCTGGGCAAAGCCACTCTGTACGCCGTGCTGGTGTCTGCC
CTGGTGCTGATGGCCATGGTCAAGCGGAAGGATAGCAGAGGC
SEQ ID NO: 10 anti-HA-1 “H” epitope TCR beta constant domain
DLKNVFPPEVAVFEPSEAEISHTQKATLVCLATGFYPDHVELSWWVNGKEVHSGVSTDPQPLKEQPALNDSRYCLSSRLRVS
ATFWQNPRNHFRCQVQFYGLSENDEWTQDRAKPVTQIVSAEAWGRADCGFTSESYQQGVLSATILYEILLGKATLYAVLVSA
LVLMAMVKRKDSRG
SEQ ID NO: 11 anti-HA-1 “H” epitope TCR beta chain
ATGGGCACAAGCCTGCTGTGTTGGATGGCCCTGTGTCTGCTGGGAGCCGATCATGCCGATACGGGAGTGTCCCAGGATCCTC
GGCACAAGATTACCAAGCGGGGCCAGAACGTGACCTTCAGATGCGACCCTATCAGCGAGCACAACCGGCTGTACTGGTACAG
ACAGACACTCGGCCAGGGACCTGAGTTCCTGACCTACTTCCAGAACGAGGCCCAACTCGAAAAGAGTAGACTGCTGAGCGAC
AGATTCAGCGCCGAAAGACCCAAGGGCAGCTTCAGCACCCTGGAAATCCAGAGAACCGAGCAGGGCGACAGCGCCATGTATC
TGTGTGCCAGCTCTCTGGTGTCCGGCAACGAGCAGTTTTTCGGCCCTGGCACCAGACTGACCGTGCTGGAAGATCTGAAGAA
CGTGTTCCCACCTGAGGTGGCCGTGTTCGAGCCTTCTGAGGCCGAGATCAGCCACACACAGAAAGCCACACTCGTGTGTCTG
GCCACCGGCTTCTATCCCGATCACGTGGAACTGTCTTGGTGGGTCAACGGCAAAGAGGTGCACAGCGGCGTCAGTACTGATC
CTCAGCCTCTCAAAGAACAGCCCGCTCTGAACGACAGCCGGTACTGTCTGAGCAGCAGACTGAGAGTGTCCGCCACCTTCTG
GCAGAATCCTAGAAATCATTTCAGGTGCCAGGTGCAATTCTATGGCCTGAGCGAAAACGATGAATGGACCCAGGATAGAGCC
AAGCCTGTGACACAAATTGTGAGCGCTGAGGCATGGGGCAGAGCCGATTGTGGCTTTACCAGCGAGAGCTACCAGCAGGGCG
TGCTGTCTGCCACAATCCTGTACGAGATCCTGCTGGGCAAAGCCACTCTGTACGCCGTGCTGGTGTCTGCCCTGGTGCTGAT
GGCCATGGTCAAGCGGAAGGATAGCAGAGGC
SEQ ID NO: 12 anti-HA-1 “H” epitope TCR beta chain
MGTSLLCWMALCLLGADHADTGVSQDPRHKITKRGQNVTFRCDPISEHNRLYWYRQTLGQGPEFLTYFQNEAQLEKSRLLSD
RFSAERPKGSFSTLEIQRTEQGDSAMYLCASSLVSGNEQFFGPGTRLTVLEDLKNVFPPEVAVFEPSEAEISHTQKATLVCL
ATGFYPDHVELSWWVNGKEVHSGVSTDPQPLKEQPALNDSRYCLSSRLRVSATFWQNPRNHERCQVQFYGLSENDEWTQDRA
KPVTQIVSAEAWGRADCGFTSESYQQGVLSATILYEILLGKATLYAVLVSALVLMAMVKRKDSRG
SEQ ID NO: 13 anti-HA-1 “H” epitope TCR alpha variable domain
ATGGCCAGCGCTCCTATTAGCATGCTGGCCATGCTGTTCACCCTGAGCGGACTGAGAGCACAGTCTGTGGCCCAGCCTGAGG
ACCAAGTGAATGTGGCCGAGGGCAATCCCCTGACCGTGAAGTGTACCTACTCCGTGTCTGGCAACCCCTACCTGTTTTGGTA
CGTGCAGTACCCCAACCGGGGCCTGCAGTTCCTGCTGAAGTACATCACCGGCGACAACCTCGTGAAGGGCTCCTATGGCTTC
GAGGCCGAATTCAACAAGAGCCAGACCAGCTTCCACCTGAAGAAACCCAGCGCACTCGTGTCCGATAGCGCCCTCTACTTTT
GCGCCGTGCGGGCTCCTACAAGCGGCACCTACAAGTACATCTTCGGCACCGGCACACGGCTGAAGGTGCTGGCCAAC
SEQ ID NO: 14 anti-HA-1 “H” epitope TCR alpha variable domain
MASAPISMLAMLFTLSGLRAQSVAQPEDQVNVAEGNPLTVKCTYSVSGNPYLFWYVQYPNRGLQFLLKYITGDNLVKGSYGF
EAEFNKSQTSFHLKKPSALVSDSALYFCAVRAPTSGTYKYIFGTGTRLKVLAN
SEQ ID NO: 15 anti-HA-1 “H” epitope TCR alpha constant domain
ATTCAGAATCCCGATCCAGCTGTCTATCAACTTCGCGATAGCAAGAGCAGCGACAAGAGCGTGTGTCTGTTTACAGACTTCG
ACTCCCAGACCAACGTGTCCCAGAGCAAGGACAGCGACGTGTACATCACAGACAAGACAGTGCTGGACATGCGGAGCATGGA
CTTCAAGAGCAACAGCGCCGTGGCCTGGTCCAACAAGTCCGATTTCGCCTGCGCCAACGCCTTCAACAACAGCATTATCCCC
GAGGACACATTCTTCCCAAGTCCTGAGAGCAGCTGCGACGTGAAGCTGGTGGAAAAGTCCTTCGAAACCGACACCAACCTGA
ATTTCCAGAACCTGAGCGTGATCGGCTTCAGAATCCTGCTGCTGAAAGTGGCCGGCTTCAACCTGCTGATGACCCTGAGACT
GTGGTCTAGC
SEQ ID NO: 16 anti-HA-1 “H” epitope TCR alpha constant domain
IQNPDPAVYQLRDSKSSDKSVCLFTDFDSQTNVSQSKDSDVYITDKTVLDMRSMDFKSNSAVAWSNKSDFACANAFNNSIIP
EDTFFPSPESSCDVKLVEKSFETDTNLNFQNLSVIGFRILLLKVAGFNLLMTLRLWSS
SEQ ID NO: 17 anti-HA-1 “H” epitope TCR alpha chain
ATGGCCAGCGCTCCTATTAGCATGCTGGCCATGCTGTTCACCCTGAGCGGACTGAGAGCACAGTCTGTGGCCCAGCCTGAGG
ACCAAGTGAATGTGGCCGAGGGCAATCCCCTGACCGTGAAGTGTACCTACTCCGTGTCTGGCAACCCCTACCTGTTTTGGTA
CGTGCAGTACCCCAACCGGGGCCTGCAGTTCCTGCTGAAGTACATCACCGGCGACAACCTCGTGAAGGGCTCCTATGGCTTC
GAGGCCGAATTCAACAAGAGCCAGACCAGCTTCCACCTGAAGAAACCCAGCGCACTCGTGTCCGATAGCGCCCTCTACTTTT
GCGCCGTGCGGGCTCCTACAAGCGGCACCTACAAGTACATCTTCGGCACCGGCACACGGCTGAAGGTGCTGGCCAACATTCA
GAATCCCGATCCAGCTGTCTATCAACTTCGCGATAGCAAGAGCAGCGACAAGAGCGTGTGTCTGTTTACAGACTTCGACTCC
CAGACCAACGTGTCCCAGAGCAAGGACAGCGACGTGTACATCACAGACAAGACAGTGCTGGACATGCGGAGCATGGACTTCA
AGAGCAACAGCGCCGTGGCCTGGTCCAACAAGTCCGATTTCGCCTGCGCCAACGCCTTCAACAACAGCATTATCCCCGAGGA
CACATTCTTCCCAAGTCCTGAGAGCAGCTGCGACGTGAAGCTGGTGGAAAAGTCCTTCGAAACCGACACCAACCTGAATTTC
CAGAACCTGAGCGTGATCGGCTTCAGAATCCTGCTGCTGAAAGTGGCCGGCTTCAACCTGCTGATGACCCTGAGACTGTGGT
CTAGC
SEQ ID NO: 18 anti-HA-1 “H” epitope TCR alpha chain
MASAPISMLAMLFTLSGLRAQSVAQPEDQVNVAEGNPLTVKCTYSVSGNPYLFWYVQYPNRGLQFLLKYITGDNLVKGSYGF
EAEFNKSQTSFHLKKPSALVSDSALYFCAVRAPTSGTYKYIFGTGTRLKVLANIQNPDPAVYQLRDSKSSDKSVCLFTDFDS
QTNVSQSKDSDVYITDKTVLDMRSMDFKSNSAVAWSNKSDFACANAFNNSIIPEDTFFPSPESSCDVKLVEKSFETDTNLNF
QNLSVIGFRILLLKVAGFNLLMTLRLWSS
SEQ ID NO: 19 anti-HA-1 “H” epitope TCR (alpha and beta chains)
ATGGGCACAAGCCTGCTGTGTTGGATGGCCCTGTGTCTGCTGGGAGCCGATCATGCCGATACGGGAGTGTCCCAGGATCCTC
GGCACAAGATTACCAAGCGGGGCCAGAACGTGACCTTCAGATGCGACCCTATCAGCGAGCACAACCGGCTGTACTGGTACAG
ACAGACACTCGGCCAGGGACCTGAGTTCCTGACCTACTTCCAGAACGAGGCCCAACTCGAAAAGAGTAGACTGCTGAGCGAC
AGATTCAGCGCCGAAAGACCCAAGGGCAGCTTCAGCACCCTGGAAATCCAGAGAACCGAGCAGGGCGACAGCGCCATGTATC
TGTGTGCCAGCTCTCTGGTGTCCGGCAACGAGCAGTTTTTCGGCCCTGGCACCAGACTGACCGTGCTGGAAGATCTGAAGAA
CGTGTTCCCACCTGAGGTGGCCGTGTTCGAGCCTTCTGAGGCCGAGATCAGCCACACACAGAAAGCCACACTCGTGTGTCTG
GCCACCGGCTTCTATCCCGATCACGTGGAACTGTCTTGGTGGGTCAACGGCAAAGAGGTGCACAGCGGCGTCAGTACTGATC
CTCAGCCTCTCAAAGAACAGCCCGCTCTGAACGACAGCCGGTACTGTCTGAGCAGCAGACTGAGAGTGTCCGCCACCTTCTG
GCAGAATCCTAGAAATCATTTCAGGTGCCAGGTGCAATTCTATGGCCTGAGCGAAAACGATGAATGGACCCAGGATAGAGCC
AAGCCTGTGACACAAATTGTGAGCGCTGAGGCATGGGGCAGAGCCGATTGTGGCTTTACCAGCGAGAGCTACCAGCAGGGCG
TGCTGTCTGCCACAATCCTGTACGAGATCCTGCTGGGCAAAGCCACTCTGTACGCCGTGCTGGTGTCTGCCCTGGTGCTGAT
GGCCATGGTCAAGCGGAAGGATAGCAGAGGCGGAAGCGGCGCCACAAACTTCAGCCTGCTTAAACAGGCCGGCGACGTGGAA
GAGAACCCCGGACCTATGGCCAGCGCTCCTATTAGCATGCTGGCCATGCTGTTCACCCTGAGCGGACTGAGAGCACAGTCTG
TGGCCCAGCCTGAGGACCAAGTGAATGTGGCCGAGGGCAATCCCCTGACCGTGAAGTGTACCTACTCCGTGTCTGGCAACCC
CTACCTGTTTTGGTACGTGCAGTACCCCAACCGGGGCCTGCAGTTCCTGCTGAAGTACATCACCGGCGACAACCTCGTGAAG
GGCTCCTATGGCTTCGAGGCCGAATTCAACAAGAGCCAGACCAGCTTCCACCTGAAGAAACCCAGCGCACTCGTGTCCGATA
GCGCCCTCTACTTTTGCGCCGTGCGGGCTCCTACAAGCGGCACCTACAAGTACATCTTCGGCACCGGCACACGGCTGAAGGT
GCTGGCCAACATTCAGAATCCCGATCCAGCTGTCTATCAACTTCGCGATAGCAAGAGCAGCGACAAGAGCGTGTGTCTGTTT
ACAGACTTCGACTCCCAGACCAACGTGTCCCAGAGCAAGGACAGCGACGTGTACATCACAGACAAGACAGTGCTGGACATGC
GGAGCATGGACTTCAAGAGCAACAGCGCCGTGGCCTGGTCCAACAAGTCCGATTTCGCCTGCGCCAACGCCTTCAACAACAG
CATTATCCCCGAGGACACATTCTTCCCAAGTCCTGAGAGCAGCTGCGACGTGAAGCTGGTGGAAAAGTCCTTCGAAACCGAC
ACCAACCTGAATTTCCAGAACCTGAGCGTGATCGGCTTCAGAATCCTGCTGCTGAAAGTGGCCGGCTTCAACCTGCTGATGA
CCCTGAGACTGTGGTCTAGC
SEQ ID NO: 20 anti-HA-1 “H” epitope TCR (alpha and beta chains)
MGTSLLCWMALCLLGADHADTGVSQDPRHKITKRGQNVTFRCDPISEHNRLYWYRQTLGQGPEFLTYFQNEAQLEKSRLLSD
RFSAERPKGSFSTLEIQRTEQGDSAMYLCASSLVSGNEQFFGPGTRLTVLEDLKNVFPPEVAVFEPSEAEISHTQKATLVCL
ATGFYPDHVELSWWVNGKEVHSGVSTDPQPLKEQPALNDSRYCLSSRLRVSATFWQNPRNHFRCQVQFYGLSENDEWTQDRA
KPVTQIVSAEAWGRADCGFTSESYQQGVLSATILYEILLGKATLYAVLVSALVLMAMVKRKDSRGGSGATNFSLLKQAGDVE
ENPGPMASAPISMLAMLFTLSGLRAQSVAQPEDQVNVAEGNPLTVKCTYSVSGNPYLFWYVQYPNRGLQFLLKYITGDNLVK
GSYGFEAEFNKSQTSFHLKKPSALVSDSALYFCAVRAPTSGTYKYIFGTGTRLKVLANIQNPDPAVYQLRDSKSSDKSVCLF
TDFDSQTNVSQSKDSDVYITDKTVLDMRSMDFKSNSAVAWSNKSDFACANAFNNSIIPEDTFFPSPESSCDVKLVEKSFETD
TNLNFQNLSVIGFRILLLKVAGFNLLMTLRLWSS
SEQ ID NO: 21 anti-HA-1 “H” epitope TCR plus RQR8 marker gene
ATGGGCACAAGCCTGCTGTGTTGGATGGCCCTGTGTCTGCTGGGAGCCGATCATGCCGATACGGGAGTGTCCCAGGATCCTC
GGCACAAGATTACCAAGCGGGGCCAGAACGTGACCTTCAGATGCGACCCTATCAGCGAGCACAACCGGCTGTACTGGTACAG
ACAGACACTCGGCCAGGGACCTGAGTTCCTGACCTACTTCCAGAACGAGGCCCAACTCGAAAAGAGTAGACTGCTGAGCGAC
AGATTCAGCGCCGAAAGACCCAAGGGCAGCTTCAGCACCCTGGAAATCCAGAGAACCGAGCAGGGCGACAGCGCCATGTATC
TGTGTGCCAGCTCTCTGGTGTCCGGCAACGAGCAGTTTTTCGGCCCTGGCACCAGACTGACCGTGCTGGAAGATCTGAAGAA
CGTGTTCCCACCTGAGGTGGCCGTGTTCGAGCCTTCTGAGGCCGAGATCAGCCACACACAGAAAGCCACACTCGTGTGTCTG
GCCACCGGCTTCTATCCCGATCACGTGGAACTGTCTTGGTGGGTCAACGGCAAAGAGGTGCACAGCGGCGTCAGTACTGATC
CTCAGCCTCTCAAAGAACAGCCCGCTCTGAACGACAGCCGGTACTGTCTGAGCAGCAGACTGAGAGTGTCCGCCACCTTCTG
GCAGAATCCTAGAAATCATTTCAGGTGCCAGGTGCAATTCTATGGCCTGAGCGAAAACGATGAATGGACCCAGGATAGAGCC
AAGCCTGTGACACAAATTGTGAGCGCTGAGGCATGGGGCAGAGCCGATTGTGGCTTTACCAGCGAGAGCTACCAGCAGGGCG
TGCTGTCTGCCACAATCCTGTACGAGATCCTGCTGGGCAAAGCCACTCTGTACGCCGTGCTGGTGTCTGCCCTGGTGCTGAT
GGCCATGGTCAAGCGGAAGGATAGCAGAGGCGGAAGCGGCGCCACAAACTTCAGCCTGCTTAAACAGGCCGGCGACGTGGAA
GAGAACCCCGGACCTATGGCCAGCGCTCCTATTAGCATGCTGGCCATGCTGTTCACCCTGAGCGGACTGAGAGCACAGTCTG
TGGCCCAGCCTGAGGACCAAGTGAATGTGGCCGAGGGCAATCCCCTGACCGTGAAGTGTACCTACTCCGTGTCTGGCAACCC
CTACCTGTTTTGGTACGTGCAGTACCCCAACCGGGGCCTGCAGTTCCTGCTGAAGTACATCACCGGCGACAACCTCGTGAAG
GGCTCCTATGGCTTCGAGGCCGAATTCAACAAGAGCCAGACCAGCTTCCACCTGAAGAAACCCAGCGCACTCGTGTCCGATA
GCGCCCTCTACTTTTGCGCCGTGCGGGCTCCTACAAGCGGCACCTACAAGTACATCTTCGGCACCGGCACACGGCTGAAGGT
GCTGGCCAACATTCAGAATCCCGATCCAGCTGTCTATCAACTTCGCGATAGCAAGAGCAGCGACAAGAGCGTGTGTCTGTTT
ACAGACTTCGACTCCCAGACCAACGTGTCCCAGAGCAAGGACAGCGACGTGTACATCACAGACAAGACAGTGCTGGACATGC
GGAGCATGGACTTCAAGAGCAACAGCGCCGTGGCCTGGTCCAACAAGTCCGATTTCGCCTGCGCCAACGCCTTCAACAACAG
CATTATCCCCGAGGACACATTCTTCCCAAGTCCTGAGAGCAGCTGCGACGTGAAGCTGGTGGAAAAGTCCTTCGAAACCGAC
ACCAACCTGAATTTCCAGAACCTGAGCGTGATCGGCTTCAGAATCCTGCTGCTGAAAGTGGCCGGCTTCAACCTGCTGATGA
CCCTGAGACTGTGGTCTAGCGGCTCTGGCGAAGGTAGAGGCAGCCTCCTTACATGCGGCGACGTCGAAGAAAATCCTGGTCC
AATGGGCACCTCTCTGCTCTGCTGGATGGCTCTTTGCCTCCTGGGTGCTGACCACGCCGATACAGCCTGTCCTTACAGCAAC
CCTAGCCTGTGTTCTGGCGGCGGAGGATCTGAACTGCCTACACAGGGCACCTTCTCCAATGTGTCCACAAATGTGTCTCCCG
CCAAGCCTACCACCACCGCTTGTCCCTACTCCAATCCATCTCTGTGCAGCGGAGGCGGCGGATCTCCTGCTCCTAGACCTCC
TACACCAGCTCCTACAATCGCCAGCCAGCCACTGTCTCTGAGGCCAGAAGCTTGTAGACCAGCTGCTGGCGGAGCCGTGCAT
ACAAGAGGACTGGACTTTGCCTGCGACATCTACATCTGGGCCCCTCTGGCTGGAACATGTGGCGTTCTGCTGCTGTCCCTGG
TCATCACCCTGTACTGCAACCACCGGAACAGGCGGAGAGTGTGCAAGTGCCCTAGACCTGTGGTT
SEQ ID NO: 22 anti-HA-1 “H” epitope TCR plus RQR8 marker gene
MGTSLLCWMALCLLGADHADTGVSQDPRHKITKRGQNVTFRCDPISEHNRLYWYRQTLGQGPEFLTYFQNEAQLEKSRLLSD
RFSAERPKGSFSTLEIQRTEQGDSAMYLCASSLVSGNEQFFGPGTRLTVLEDLKNVFPPEVAVFEPSEAEISHTQKATLVCL
ATGFYPDHVELSWWVNGKEVHSGVSTDPQPLKEQPALNDSRYCLSSRLRVSATFWQNPRNHERCQVQFYGLSENDEWTQDRA
KPVTQIVSAEAWGRADCGFTSESYQQGVLSATILYEILLGKATLYAVLVSALVLMAMVKRKDSRGGSGATNFSLLKQAGDVE
ENPGPMASAPISMLAMLFTLSGLRAQSVAQPEDQVNVAEGNPLTVKCTYSVSGNPYLFWYVQYPNRGLQFLLKYITGDNLVK
GSYGFEAEFNKSQTSFHLKKPSALVSDSALYFCAVRAPTSGTYKYIFGTGTRLKVLANIQNPDPAVYQLRDSKSSDKSVCLF
TDFDSQTNVSQSKDSDVYITDKTVLDMRSMDFKSNSAVAWSNKSDFACANAFNNSIIPEDTFFPSPESSCDVKLVEKSFETD
TNLNFQNLSVIGFRILLLKVAGFNLLMTLRLWSSGSGEGRGSLLTCGDVEENPGPMGTSLLCWMALCLLGADHADTACPYSN
PSLCSGGGGSELPTQGTFSNVSTNVSPAKPTTTACPYSNPSLCSGGGGSPAPRPPTPAPTIASQPLSLRPEACRPAAGGAVH
TRGLDFACDIYIWAPLAGTCGVLLLSLVITLYCNHRNRRRVCKCPRPVV
SEQ ID NO: 23 anti-HA-1 “H” epitope TCR plus RQR8 marker gene (with promoter
and kozak)
CGATTAGTCCAATTTGTTAAAGACAGGATATCAGTGGTCCAGGCTCTAGTTTTGACTCAACAATATCACCAGCTGAAGCCTA
TAGAGTACGAGCCATAGATAGAATAAAAGATTTTATTTAGTCTCCAGAAAAAGGGGGGAATGAAAGACCCCACCTGTAGGTT
TGGCAAGCTAGGATCAAGGTTAGGAACAGAGAGACAGCAGAATATGGGCCAAACAGGATATCTGTGGTAAGCAGTTCCTGCC
CCGGCTCAGGGCCAAGAACAGTTGGAACAGCAGAATATGGGCCAAACAGGATATCTGTGGTAAGCAGTTCCTGCCCCGGCTC
AGGGCCAAGAACAGATGGTCCCCAGATGCGGTCCCGCCCTCAGCAGTTTCTAGAGAACCATCAGATGTTTCCAGGGTGCCCC
AAGGACCTGAAATGACCCTGTGCCTTATTTGAACTAACCAATCAGTTCGCTTCTCGCTTCTGTTCGCGCGCTTCTGCTCCCC
GAGCTCAATAAAAGAGCCCACAACCCCTCACTCGGCGCGATCTAGATCTCGAATCTCTAGAGCCGCCACCATGGGCACAAGC
CTGCTGTGTTGGATGGCCCTGTGTCTGCTGGGAGCCGATCATGCCGATACGGGAGTGTCCCAGGATCCTCGGCACAAGATTA
CCAAGCGGGGCCAGAACGTGACCTTCAGATGCGACCCTATCAGCGAGCACAACCGGCTGTACTGGTACAGACAGACACTCGG
CCAGGGACCTGAGTTCCTGACCTACTTCCAGAACGAGGCCCAACTCGAAAAGAGTAGACTGCTGAGCGACAGATTCAGCGCC
GAAAGACCCAAGGGCAGCTTCAGCACCCTGGAAATCCAGAGAACCGAGCAGGGCGACAGCGCCATGTATCTGTGTGCCAGCT
CTCTGGTGTCCGGCAACGAGCAGTTTTTCGGCCCTGGCACCAGACTGACCGTGCTGGAAGATCTGAAGAACGTGTTCCCACC
TGAGGTGGCCGTGTTCGAGCCTTCTGAGGCCGAGATCAGCCACACACAGAAAGCCACACTCGTGTGTCTGGCCACCGGCTTC
TATCCCGATCACGTGGAACTGTCTTGGTGGGTCAACGGCAAAGAGGTGCACAGCGGCGTCAGTACTGATCCTCAGCCTCTCA
AAGAACAGCCCGCTCTGAACGACAGCCGGTACTGTCTGAGCAGCAGACTGAGAGTGTCCGCCACCTTCTGGCAGAATCCTAG
AAATCATTTCAGGTGCCAGGTGCAATTCTATGGCCTGAGCGAAAACGATGAATGGACCCAGGATAGAGCCAAGCCTGTGACA
CAAATTGTGAGCGCTGAGGCATGGGGCAGAGCCGATTGTGGCTTTACCAGCGAGAGCTACCAGCAGGGCGTGCTGTCTGCCA
CAATCCTGTACGAGATCCTGCTGGGCAAAGCCACTCTGTACGCCGTGCTGGTGTCTGCCCTGGTGCTGATGGCCATGGTCAA
GCGGAAGGATAGCAGAGGCGGAAGCGGCGCCACAAACTTCAGCCTGCTTAAACAGGCCGGCGACGTGGAAGAGAACCCCGGA
CCTATGGCCAGCGCTCCTATTAGCATGCTGGCCATGCTGTTCACCCTGAGCGGACTGAGAGCACAGTCTGTGGCCCAGCCTG
AGGACCAAGTGAATGTGGCCGAGGGCAATCCCCTGACCGTGAAGTGTACCTACTCCGTGTCTGGCAACCCCTACCTGTTTTG
GTACGTGCAGTACCCCAACCGGGGCCTGCAGTTCCTGCTGAAGTACATCACCGGCGACAACCTCGTGAAGGGCTCCTATGGC
TTCGAGGCCGAATTCAACAAGAGCCAGACCAGCTTCCACCTGAAGAAACCCAGCGCACTCGTGTCCGATAGCGCCCTCTACT
TTTGCGCCGTGCGGGCTCCTACAAGCGGCACCTACAAGTACATCTTCGGCACCGGCACACGGCTGAAGGTGCTGGCCAACAT
TCAGAATCCCGATCCAGCTGTCTATCAACTTCGCGATAGCAAGAGCAGCGACAAGAGCGTGTGTCTGTTTACAGACTTCGAC
TCCCAGACCAACGTGTCCCAGAGCAAGGACAGCGACGTGTACATCACAGACAAGACAGTGCTGGACATGCGGAGCATGGACT
TCAAGAGCAACAGCGCCGTGGCCTGGTCCAACAAGTCCGATTTCGCCTGCGCCAACGCCTTCAACAACAGCATTATCCCCGA
GGACACATTCTTCCCAAGTCCTGAGAGCAGCTGCGACGTGAAGCTGGTGGAAAAGTCCTTCGAAACCGACACCAACCTGAAT
TTCCAGAACCTGAGCGTGATCGGCTTCAGAATCCTGCTGCTGAAAGTGGCCGGCTTCAACCTGCTGATGACCCTGAGACTGT
GGTCTAGCGGCTCTGGCGAAGGTAGAGGCAGCCTCCTTACATGCGGCGACGTCGAAGAAAATCCTGGTCCAATGGGCACCTC
TCTGCTCTGCTGGATGGCTCTTTGCCTCCTGGGTGCTGACCACGCCGATACAGCCTGTCCTTACAGCAACCCTAGCCTGTGT
TCTGGCGGCGGAGGATCTGAACTGCCTACACAGGGCACCTTCTCCAATGTGTCCACAAATGTGTCTCCCGCCAAGCCTACCA
CCACCGCTTGTCCCTACTCCAATCCATCTCTGTGCAGCGGAGGCGGCGGATCTCCTGCTCCTAGACCTCCTACACCAGCTCC
TACAATCGCCAGCCAGCCACTGTCTCTGAGGCCAGAAGCTTGTAGACCAGCTGCTGGCGGAGCCGTGCATACAAGAGGACTG
GACTTTGCCTGCGACATCTACATCTGGGCCCCTCTGGCTGGAACATGTGGCGTTCTGCTGCTGTCCCTGGTCATCACCCTGT
ACTGCAACCACCGGAACAGGCGGAGAGTGTGCAAGTGCCCTAGACCTGTGGTT
SEQ ID NO: 24 Plasmid for generating lentiviral vector
GGGTCTCTCTGGTTAGACCAGATCTGAGCCTGGGAGCTCTCTGGCTAACTAGGGAACCCACTGCTTAAGCCTCAATAAAGCT
TGCCTTGAGTGCTTCAAGTAGTGTGTGCCCGTCTGTTGTGTGACTCTGGTAACTAGAGATCCCTCAGACCCTTTTAGTCAGT
GTGGAAAATCTCTAGCAGTGGCGCCCGAACAGGGACTTGAAAGCGAAAGGGAAACCAGAGGAGCTCTCTCGACGCAGGACTC
GGCTTGCTGAAGCGCGCACGGCAAGAGGCGAGGGGCGGCGACTGGTGAGTACGCCAAAAATTTTGACTAGCGGAGGCTAGAA
GGAGAGAGATGGGTGCGAGAGCGTCAGTATTAAGCGGGGGAGAATTAGATCGCGATGGGAAAAAATTCGGTTAAGGCCAGGG
GGAAAGAAAAAATATAAATTAAAACATATAGTATGGGCAAGCAGGGAGCTAGAACGATTCGCAGTTAATCCTGGCCTGTTAG
AAACATCAGAAGGCTGTAGACAAATACTGGGACAGCTACAACCATCCCTTCAGACAGGATCAGAAGAACTTAGATCATTATA
TAATACAGTAGCAACCCTCTATTGTGTGCATCAAAGGATAGAGATAAAAGACACCAAGGAAGCTTTAGACAAGATAGAGGAA
GAGCAAAACAAAAGTAAGACCACCGCACAGCAAGCGGCCGCTGATCTTCAGACCTGGAGGAGGAGATATGAGGGACAATTGG
AGAAGTGAATTATATAAATATAAAGTAGTAAAAATTGAACCATTAGGAGTAGCACCCACCAAGGCAAAGAGAAGAGTGGTGC
AGAGAGAAAAAAGAGCAGTGGGAATAGGAGCTTTGTTCCTTGGGTTCTTGGGAGCAGCAGGAAGCACTATGGGCGCAGCGTC
AATGACGCTGACGGTACAGGCCAGACAATTATTGTCTGGTATAGTGCAGCAGCAGAACAATTTGCTGAGGGCTATTGAGGCG
CAACAGCATCTGTTGCAACTCACAGTCTGGGGCATCAAGCAGCTCCAGGCAAGAATCCTGGCTGTGGAAAGATACCTAAAGG
ATCAACAGCTCCTGGGGATTTGGGGTTGCTCTGGAAAACTCATTTGCACCACTGCTGTGCCTTGGAATGCTAGTTGGAGTAA
TAAATCTCTGGAACAGATTTGGAATCACACGACCTGGATGGAGTGGGACAGAGAAATTAACAATTACACAAGCTTAATACAC
TCCTTAATTGAAGAATCGCAAAACCAGCAAGAAAAGAATGAACAAGAATTATTGGAATTAGATAAATGGGCAAGTTTGTGGA
ATTGGTTTAACATAACAAATTGGCTGTGGTATATAAAATTATTCATAATGATAGTAGGAGGCTTGGTAGGTTTAAGAATAGT
TTTTGCTGTACTTTCTATAGTGAATAGAGTTAGGCAGGGATATTCACCATTATCGTTTCAGACCCACCTCCCAACCCCGAGG
GGACCCGACAGGCCCGAAGGAATAGAAGAAGAAGGTGGAGAGAGAGACAGAGACAGATCCATTCGATTAGTGAACGGATCTC
GACGGTATCGATTAGACTGTAGCCCAGGAATATGGCAGCTAGATTGTACACATTTAGAAGGAAAAGTTATCTTGGTAGCAGT
TCATGTAGCCAGTGGATATATAGAAGCAGAAGTAATTCCAGCAGAGACAGGGCAAGAAACAGCATACTTCCTCTTAAAATTA
GCAGGAAGATGGCCAGTAAAAACAGTACATACAGACAATGGCAGCAATTTCACCAGTACTACAGTTAAGGCCGCCTGTTGGT
GGGCGGGGATCAAGCAGGAATTTGGCATTCCCTACAATCCCCAAAGTCAAGGAGTAATAGAATCTATGAATAAAGAATTAAA
GAAAATTATAGGACAGGTAAGAGATCAGGCTGAACATCTTAAGACAGCAGTACAAATGGCAGTATTCATCCACAATTTTAAA
AGAAAAGGGGGGATTGGGGGGTACAGTGCAGGGGAAAGAATAGTAGACATAATAGCAACAGACATACAAACTAAAGAATTAC
AAAAACAAATTACAAAAATTCAAAATTTTCGGGTTTATTACAGGGACAGCAGAGATCCAGTTTGGCTGCATTGATCACGTGA
GGGGCGATTAGTCCAATTTGTTAAAGACAGGATATCAGTGGTCCAGGCTCTAGTTTTGACTCAACAATATCACCAGCTGAAG
CCTATAGAGTACGAGCCATAGATAGAATAAAAGATTTTATTTAGTCTCCAGAAAAAGGGGGGAATGAAAGACCCCACCTGTA
GGTTTGGCAAGCTAGGATCAAGGTTAGGAACAGAGAGACAGCAGAATATGGGCCAAACAGGATATCTGTGGTAAGCAGTTCC
TGCCCCGGCTCAGGGCCAAGAACAGTTGGAACAGCAGAATATGGGCCAAACAGGATATCTGTGGTAAGCAGTTCCTGCCCCG
GCTCAGGGCCAAGAACAGATGGTCCCCAGATGCGGTCCCGCCCTCAGCAGTTTCTAGAGAACCATCAGATGTTTCCAGGGTG
CCCCAAGGACCTGAAATGACCCTGTGCCTTATTTGAACTAACCAATCAGTTCGCTTCTCGCTTCTGTTCGCGCGCTTCTGCT
CCCCGAGCTCAATAAAAGAGCCCACAACCCCTCACTCGGCGCGATCTAGATCTCGAATCTCTAGAGCCGCCACCATGGGCAC
AAGCCTGCTGTGTTGGATGGCCCTGTGTCTGCTGGGAGCCGATCATGCCGATACGGGAGTGTCCCAGGATCCTCGGCACAAG
ATTACCAAGCGGGGCCAGAACGTGACCTTCAGATGCGACCCTATCAGCGAGCACAACCGGCTGTACTGGTACAGACAGACAC
TCGGCCAGGGACCTGAGTTCCTGACCTACTTCCAGAACGAGGCCCAACTCGAAAAGAGTAGACTGCTGAGCGACAGATTCAG
CGCCGAAAGACCCAAGGGCAGCTTCAGCACCCTGGAAATCCAGAGAACCGAGCAGGGCGACAGCGCCATGTATCTGTGTGCC
AGCTCTCTGGTGTCCGGCAACGAGCAGTTTTTCGGCCCTGGCACCAGACTGACCGTGCTGGAAGATCTGAAGAACGTGTTCC
CACCTGAGGTGGCCGTGTTCGAGCCTTCTGAGGCCGAGATCAGCCACACACAGAAAGCCACACTCGTGTGTCTGGCCACCGG
CTTCTATCCCGATCACGTGGAACTGTCTTGGTGGGTCAACGGCAAAGAGGTGCACAGCGGCGTCAGTACTGATCCTCAGCCT
CTCAAAGAACAGCCCGCTCTGAACGACAGCCGGTACTGTCTGAGCAGCAGACTGAGAGTGTCCGCCACCTTCTGGCAGAATC
CTAGAAATCATTTCAGGTGCCAGGTGCAATTCTATGGCCTGAGCGAAAACGATGAATGGACCCAGGATAGAGCCAAGCCTGT
GACACAAATTGTGAGCGCTGAGGCATGGGGCAGAGCCGATTGTGGCTTTACCAGCGAGAGCTACCAGCAGGGCGTGCTGTCT
GCCACAATCCTGTACGAGATCCTGCTGGGCAAAGCCACTCTGTACGCCGTGCTGGTGTCTGCCCTGGTGCTGATGGCCATGG
TCAAGCGGAAGGATAGCAGAGGCGGAAGCGGCGCCACAAACTTCAGCCTGCTTAAACAGGCCGGCGACGTGGAAGAGAACCC
CGGACCTATGGCCAGCGCTCCTATTAGCATGCTGGCCATGCTGTTCACCCTGAGCGGACTGAGAGCACAGTCTGTGGCCCAG
CCTGAGGACCAAGTGAATGTGGCCGAGGGCAATCCCCTGACCGTGAAGTGTACCTACTCCGTGTCTGGCAACCCCTACCTGT
TTTGGTACGTGCAGTACCCCAACCGGGGCCTGCAGTTCCTGCTGAAGTACATCACCGGCGACAACCTCGTGAAGGGCTCCTA
TGGCTTCGAGGCCGAATTCAACAAGAGCCAGACCAGCTTCCACCTGAAGAAACCCAGCGCACTCGTGTCCGATAGCGCCCTC
TACTTTTGCGCCGTGCGGGCTCCTACAAGCGGCACCTACAAGTACATCTTCGGCACCGGCACACGGCTGAAGGTGCTGGCCA
ACATTCAGAATCCCGATCCAGCTGTCTATCAACTTCGCGATAGCAAGAGCAGCGACAAGAGCGTGTGTCTGTTTACAGACTT
CGACTCCCAGACCAACGTGTCCCAGAGCAAGGACAGCGACGTGTACATCACAGACAAGACAGTGCTGGACATGCGGAGCATG
GACTTCAAGAGCAACAGCGCCGTGGCCTGGTCCAACAAGTCCGATTTCGCCTGCGCCAACGCCTTCAACAACAGCATTATCC
CCGAGGACACATTCTTCCCAAGTCCTGAGAGCAGCTGCGACGTGAAGCTGGTGGAAAAGTCCTTCGAAACCGACACCAACCT
GAATTTCCAGAACCTGAGCGTGATCGGCTTCAGAATCCTGCTGCTGAAAGTGGCCGGCTTCAACCTGCTGATGACCCTGAGA
CTGTGGTCTAGCGGCTCTGGCGAAGGTAGAGGCAGCCTCCTTACATGCGGCGACGTCGAAGAAAATCCTGGTCCAATGGGCA
CCTCTCTGCTCTGCTGGATGGCTCTTTGCCTCCTGGGTGCTGACCACGCCGATACAGCCTGTCCTTACAGCAACCCTAGCCT
GTGTTCTGGCGGCGGAGGATCTGAACTGCCTACACAGGGCACCTTCTCCAATGTGTCCACAAATGTGTCTCCCGCCAAGCCT
ACCACCACCGCTTGTCCCTACTCCAATCCATCTCTGTGCAGCGGAGGCGGCGGATCTCCTGCTCCTAGACCTCCTACACCAG
CTCCTACAATCGCCAGCCAGCCACTGTCTCTGAGGCCAGAAGCTTGTAGACCAGCTGCTGGCGGAGCCGTGCATACAAGAGG
ACTGGACTTTGCCTGCGACATCTACATCTGGGCCCCTCTGGCTGGAACATGTGGCGTTCTGCTGCTGTCCCTGGTCATCACC
CTGTACTGCAACCACCGGAACAGGCGGAGAGTGTGCAAGTGCCCTAGACCTGTGGTTTAAGTAGGTGAGTTCGACAATCAAC
CTCTGGATTACAAAATTTGTGAAAGATTGACTGGTATTCTTAACTATGTTGCTCCTTTTACGCTATGTGGATACGCTGCTTT
AATGCCTTTGTATCATGCTATTGCTTCCCGTATGGCTTTCATTTTCTCCTCCTTGTATAAATCCTGGTTGCTGTCTCTTTAT
GAGGAGTTGTGGCCCGTTGTCAGGCAACGTGGCGTGGTGTGCACTGTGTTTGCTGACGCAACCCCCACTGGTTGGGGCATTG
CCACCACCTGTCAGCTCCTTTCCGGGACTTTCGCTTTCCCCCTCCCTATTGCCACGGCGGAACTCATCGCCGCCTGCCTTGC
CCGCTGCTGGACAGGGGCTCGGCTGTTGGGCACTGACAATTCCGTGGTGTTGTCGGGGAAGCTGACGTCCTTTCCATGGCTG
CTCGCCTGTGTTGCCACCTGGATTCTGCGCGGGACGTCCTTCTGCTACGTCCCTTCGGCCCTCAATCCAGCGGACCTTCCTT
CCCGCGGCCTGCTGCCGGCTCTGCGGCCTCTTCCGCGTCTTCGCCTTCGCCCTCAGACGAGTCGGATCTCCCTTTGGGCCGC
CTCCCCGCGAATTCGAGCTCGGTACCTTTAAGACCAATGACTTACAAGGCAGCTGTAGATCTTAGCCACTTTTTAAAAGAAA
AGGGGGGACTGGAAGGGCTAATTCACTCCCAACGAAGACAAGATCTGCTTTTTGCTTGTACTGGGTCTCTCTGGTTAGACCA
GATCTGAGCCTGGGAGCTCTCTGGCTAACTAGGGAACCCACTGCTTAAGCCTCAATAAAGCTTGCCTTGAGTGCTTCAAGTA
GTGTGTGCCCGTCTGTTGTGTGACTCTGGTAACTAGAGATCCCTCAGACCCTTTTAGTCAGTGTGGAAAATCTCTAGCAGTA
GTAGTTCATGTCATCTTATTATTCAGTATTTATAACTTGCAAAGAAATGAATATCAGAGAGTGAGAGGAACTTGTTTATTGC
AGCTTATAATGGTTACAAATAAAGCAATAGCATCACAAATTTCACAAATAAAGCATTTTTTTCACTGCATTCTAGTTGTGGT
TTGTCCAAACTCATCAATGTATCTTATCATGTCTGGCTCTAGCTATCCCGCCCCTAACTCCGCCCATCCCGCCCCTAACTCC
GCCCAGTTCCGCCCATTCTCCGCCCCATGGCTGACTAATTTTTTTTATTTATGCAGAGGCCGAGGCCGCCTCGGCCTCTGAG
CTATTCCAGAAGTAGTGAGGAGGCTTTTTTGGAGGCCTAGGGACGTACGTCAAAAGGGCGACACAAAATTTATTCTAAATGC
ATAATAAATACTGATAACATCTTATAGTTTGTATTATATTTTGTATTATCGTTGACATGTATAATTTTGATATCAAAAACTG
ATTTTCCCTTTATTATTTTCGAGATTTATTTTCTTAATTCTCTTTAACAAACTAGAAATATTGTATATACAAAAAATCATAA
ATAATAGATGAATAGTTTAATTATAGGTGTTCATCAATCGAAAAAGCAACGTATCTTATTTAAAGTGCGTTGCTTTTTTCTC
ATTTATAAGGTTAAATAATTCTCATATATCAAGCAAAGTGACAGGCGCCCTTAAATATTCTGACAAATGCTCTTTCCCTAAA
CTCCCCCCATAAAAAAACCCGCCGAAGCGGGTTTTTACGTTATTTGCGGATTAACGATTACTCGTTATCAGAACCGCCCAGG
GGGCCCGAGCTTAAGACTGGCCGTCGTTTTACAACACAGAAAGAGTTTGTAGAAACGCAAAAAGGCCATCCGTCAGGGGCCT
TCTGCTTAGTTTGATGCCTGGCAGTTCCCTACTCTCGCCTTCCGCTTCCTCGCTCACTGACTCGCTGCGCTCGGTCGTTCGG
CTGCGGCGAGCGGTATCAGCTCACTCAAAGGCGGTAATACGGTTATCCACAGAATCAGGGGATAACGCAGGAAAGAACATGT
GAGCAAAAGGCCAGCAAAAGGCCAGGAACCGTAAAAAGGCCGCGTTGCTGGCGTTTTTCCATAGGCTCCGCCCCCCTGACGA
GCATCACAAAAATCGACGCTCAAGTCAGAGGTGGCGAAACCCGACAGGACTATAAAGATACCAGGCGTTTCCCCCTGGAAGC
TCCCTCGTGCGCTCTCCTGTTCCGACCCTGCCGCTTACCGGATACCTGTCCGCCTTTCTCCCTTCGGGAAGCGTGGCGCTTT
CTCATAGCTCACGCTGTAGGTATCTCAGTTCGGTGTAGGTCGTTCGCTCCAAGCTGGGCTGTGTGCACGAACCCCCCGTTCA
GCCCGACCGCTGCGCCTTATCCGGTAACTATCGTCTTGAGTCCAACCCGGTAAGACACGACTTATCGCCACTGGCAGCAGCC
ACTGGTAACAGGATTAGCAGAGCGAGGTATGTAGGCGGTGCTACAGAGTTCTTGAAGTGGTGGGCTAACTACGGCTACACTA
GAAGAACAGTATTTGGTATCTGCGCTCTGCTGAAGCCAGTTACCTTCGGAAAAAGAGTTGGTAGCTCTTGATCCGGCAAACA
AACCACCGCTGGTAGCGGTGGTTTTTTTGTTTGCAAGCAGCAGATTACGCGCAGAAAAAAAGGATCTCAAGAAGATCCTTTG
ATCTTTTCTACGGGGTCTGACGCTCAGTGGAACGACGCGCGCGTAACTCACGTTAAGGGATTTTGGTCATGAGCTTGCGCCG
TCCCGTCAAGTCAGCGTAATGCTCTGCTTTTAGAAAAACTCATCGAGCATCAAATGAAACTGCAATTTATTCATATCAGGAT
TATCAATACCATATTTTTGAAAAAGCCGTTTCTGTAATGAAGGAGAAAACTCACCGAGGCAGTTCCATAGGATGGCAAGATC
CTGGTATCGGTCTGCGATTCCGACTCGTCCAACATCAATACAACCTATTAATTTCCCCTCGTCAAAAATAAGGTTATCAAGT
GAGAAATCACCATGAGTGACGACTGAATCCGGTGAGAATGGCAAAAGTTTATGCATTTCTTTCCAGACTTGTTCAACAGGCC
AGCCATTACGCTCGTCATCAAAATCACTCGCATCAACCAAACCGTTATTCATTCGTGATTGCGCCTGAGCGAGGCGAAATAC
GCGATCGCTGTTAAAAGGACAATTACAAACAGGAATCGAGTGCAACCGGCGCAGGAACACTGCCAGCGCATCAACAATATTT
TCACCTGAATCAGGATATTCTTCTAATACCTGGAACGCTGTTTTTCCGGGGATCGCAGTGGTGAGTAACCATGCATCATCAG
GAGTACGGATAAAATGCTTGATGGTCGGAAGTGGCATAAATTCCGTCAGCCAGTTTAGTCTGACCATCTCATCTGTAACATC
ATTGGCAACGCTACCTTTGCCATGTTTCAGAAACAACTCTGGCGCATCGGGCTTCCCATACAAGCGATAGATTGTCGCACCT
GATTGCCCGACATTATCGCGAGCCCATTTATACCCATATAAATCAGCATCCATGTTGGAATTTAATCGCGGCCTCGACGTTT
CCCGTTGAATATGGCTCATATTCTTCCTTTTTCAATATTATTGAAGCATTTATCAGGGTTATTGTCTCATGAGCGGATACAT
ATTTGAATGTATTTAGAAAAATAAACAAATAGGGGTCAGTGTTACAACCAATTAACCAATTCTGAACATTATCGCGAGCCCA
TTTATACCTGAATATGGCTCATAACACCCCTTGTTTGCCTGGCGGCAGTAGCGCGGTGGTCCCACCTGACCCCATGCCGAAC
TCAGAAGTGAAACGCCGTAGCGCCGATGGTAGTGTGGGGACTCCCCATGCGAGAGTAGGGAACTGCCAGGCATCAAATAAAA
CGAAAGGCTCAGTCGAAAGACTGGGCCTTTCGCCCGGGCTAATTAGGGGGTGTCGCCCTTCGCTGAAAGCTGCAAGCTTAAT
GTAGTCTTATGCAATACTCTTGTAGTCTTGCAACATGGTAACGATGAGTTAGCAACATGCCTTACAAGGAGAGAAAAAGCAC
CGTGCATGCCGATTGGTGGAAGTAAGGTGGTACGATCGTGCCTTATTAGGAAGGCAACAGACGGGTCTGACATGGATTGGAC
GAACCACTGAATTGCCGCATTGCAGAGATATTGTATTTAAGTGCCTAGCTCGATACATAAAC

TABLE 3B
Anti-HA-2 “V” epitope TCR (anti-HA-2 TCR) alpha and beta variable region and
alpha and beta chain amino acid sequences

| SEQ | | |
|---|---|---|
| ID | | |
| NO | Sequence | Description |
| 25 | METLLGLLILWLQLQWVSSKQEVTQIPAALSVPEGENLVLNCSFTDSAIYNLQWFRQDPGKGLTSL | TCR A Vα |
| | LLIQSSQREQTSGRLNASLDKSSGRSTLYIAASQPGDSATYLCAVRPKNYGGSQGNLIFGKGTKLS | (aa) |
| | VKPN | |
| 26 | METLLGLLILWLQLQWVSSKQEVTQIPAALSVPEGENLVLNCSFTDSAIYNLQWFRQDPGKGLTSL | TCR A Vα + |
| | LLIQSSQREQTSGRLNASLDKSSGRSTLYIAASQPGDSATYLCAVRPKNYGGSQGNLIFGKGTKLS | Cα (aa) |
| | VKPNIQNPDPAVYQLRDSKSSDKSVCLFTDFDSQTNVSQSKDSDVYITDKTVLDMRSMDFKSNSAV | |
| | AWSNKSDFACANAFNNSIIPEDTFFPSPESSCDVKLVEKSFETDTNLNFQNLSVIGFRILLLKVAG | |
| | FNLLMTLRLWSS | |
| 27 | MGTRLLCWVVLGFLGTDHTGAGVSQSPRYKVAKRGQDVALRCDPISGHVSLFWYQQALGQGPEFLT | TCR A Vβ |
| | YFQNEAQLDKSGLPSDRFFAERPEGSVSTLKIQRTQQEDSAVYLCASSFPGLASYEQYFGPGTRLT | (aa) |
| | VTE | |
| 28 | MGTRLLCWVVLGFLGTDHTGAGVSQSPRYKVAKRGQDVALRCDPISGHVSLFWYQQALGQGPEFLT | TCR A Vβ + |
| | YFQNEAQLDKSGLPSDRFFAERPEGSVSTLKIQRTQQEDSAVYLCASSFPGLASYEQYFGPGTRLT | Cβ (aa) |
| | VTEDLKNVFPPEVAVFEPSEAEISHTQKATLVCLATGFYPDHVELSWWVNGKEVHSGVSTDPQPLK | |
| | EQPALNDSRYCLSSRLRVSATFWQNPRNHERCQVQFYGLSENDEWTQDRAKPVTQIVSAEAWGRAD | |
| | CGFTSESYQQGVLSATILYEILLGKATLYAVLVSALVLMAMVKRKDSRG | |
| 29 | METLLGLLILWLQLQWVSSKQEVTQIPAALSVPEGENLVLNCSFTDSAIYNLQWFRQDPGKGLTSL | TCR B Vα |
| | LLIQSSQREQTSGRLNASLDKSSGRSTLYIAASQPGDSATYLCAVRQGYGGSQGNLIFGKGTKLSV | (aa) |
| | KPN | |
| 30 | METLLGLLILWLQLQWVSSKQEVTQIPAALSVPEGENLVLNCSFTDSAIYNLQWFRQDPGKGLTSL | TCR B Vα + |
| | LLIQSSQREQTSGRLNASLDKSSGRSTLYIAASQPGDSATYLCAVRQGYGGSQGNLIFGKGTKLSV | Cα (aa) |
| | KPNIQNPDPAVYQLRDSKSSDKSVCLFTDFDSQTNVSQSKDSDVYITDKTVLDMRSMDFKSNSAVA | |
| | WSNKSDFACANAFNNSIIPEDTFFPSPESSCDVKLVEKSFETDTNLNFQNLSVIGFRILLLKVAGF | |
| | NLLMTLRLWSS | |
| 31 | MGTRLLCWVVLGFLGTDHTGAGVSQSPRYKVAKRGQDVALRCDPISGHVSLFWYQQALGQGPEFLT | TCR B Vβ |
| | YFQNEAQLDKSGLPSDRFFAERPEGSVSTLKIQRTQQEDSAVYLCASSLIQGTNSPLHFGNGTRLT | (aa) |
| | VTE | |
| 32 | MGTRLLCWVVLGFLGTDHTGAGVSQSPRYKVAKRGQDVALRCDPISGHVSLFWYQQALGQGPEFLT | TCR B Vβ + |
| | YFQNEAQLDKSGLPSDRFFAERPEGSVSTLKIQRTQQEDSAVYLCASSLIQGTNSPLHEGNGTRLT | Cβ (aa) |
| | VTEDLNKVFPPEVAVFEPSEAEISHTQKATLVCLATGFFPDHVELSWWVNGKEVHSGVSTDPQPLK | |
| | EQPALNDSRYCLSSRLRVSATFWQNPRNHFRCQVQFYGLSENDEWTQDRAKPVTQIVSAEAWGRAD | |
| | CGFTSVSYQQGVLSATILYEILLGKATLYAVLVSALVLMAMVKRKDF | |
| 33 | MEKMLECAFIVLWLQLGWLSGEDQVTQSPEALRLQEGESSSLNCSYTVSGLRGLFWYRQDPGKGPE | TCR C Vα |
| | FLFTLYSAGEEKEKERLKATLTKKESFLHITAPKPEDSATYLCAVQAWDYGGSQGNLIFGKGTKLS | (aa) |
| | VKPN | |
| 34 | MEKMLECAFIVLWLQLGWLSGEDQVTQSPEALRLQEGESSSLNCSYTVSGLRGLFWYRQDPGKGPE | TCR C Vα + |
| | FLFTLYSAGEEKEKERLKATLTKKESFLHITAPKPEDSATYLCAVQAWDYGGSQGNLIFGKGTKLS | Cα (aa) |
| | VKPNIQNPDPAVYQLRDSKSSDKSVCLFTDFDSQTNVSQSKDSDVYITDKTVLDMRSMDFKSNSAV | |
| | AWSNKSDFACANAFNNSIIPEDTFFPSPESSCDVKLVEKSFETDTNLNFQNLSVIGFRILLLKVAG | |
| | FNLLMTLRLWSS | |
| 35 | MDTRVLCCAVICLLGAGLSNAGVMQNPRHLVRRRGQEARLRCSPMKGHSHVYWYRQLPEEGLKFMV | TCR C Vβ |
| | YLQKENIIDESGMPKERFSAEFPKEGPSILRIQQVVRGDSAAYFCASSTREGTDTQYFGPGTRLTV | (aa) |
| | LE | |
| 36 | MDTRVLCCAVICLLGAGLSNAGVMQNPRHLVRRRGQEARLRCSPMKGHSHVYWYRQLPEEGLKFMV | TCR C Vβ + |
| | YLQKENIIDESGMPKERFSAEFPKEGPSILRIQQVVRGDSAAYFCASSTREGTDTQYFGPGTRLTV | Cβ (aa) |
| | LEDLKNVFPPEVAVFEPSEAEISHTQKATLVCLATGFYPDHVELSWWVNGKEVHSGVSTDPQPLKE | |
| | QPALNDSRYCLSSRLRVSATFWQNPRNHFRCQVQFYGLSENDEWTQDRAKPVTQIVSAEAWGRADC | |
| | GFTSESYQQGVLSATILYEILLGKATLYAVLVSALVLMAMVKRKDSRG | |
| 37 | METLLGLLILWLQLQWVSSKQEVTQIPAALSVPEGENLVLNCSFTDSAIYNLQWFRQDPGKGLTSL | TCR D Vα |
| | LLIQSSQREQTSGRLNASLDKSSGRSTLYIAASQPGDSATYLCAVRRGYGGSQGNLIFGKGTKLSV | (aa) |
| | KPN | |
| 38 | METLLGLLILWLQLQWVSSKQEVTQIPAALSVPEGENLVLNCSFTDSAIYNLQWFRQDPGKGLTSL | TCR D Vα + |
| | LLIQSSQREQTSGRLNASLDKSSGRSTLYIAASQPGDSATYLCAVRRGYGGSQGNLIFGKGTKLSV | Cα (aa) |
| | KPNIQNPDPAVYQLRDSKSSDKSVCLFTDFDSQTNVSQSKDSDVYITDKTVLDMRSMDFKSNSAVA | |
| | WSNKSDFACANAFNNSIIPEDTFFPSPESSCDVKLVEKSFETDTNLNFQNLSVIGFRILLLKVAGF | |
| | NLLMTLRLWSS | |
| 39 | MGTRLLCWVVLGFLGTDHTGAGVSQSPRYKVAKRGQDVALRCDPISGHVSLFWYQQALGQGPEFLT | TCR D Vβ |
| | YFQNEAQLDKSGLPSDRFFAERPEGSVSTLKIQRTQQEDSAVYLCASSLTAGAYNEQFFGPGTRLT | (aa) |
| | VLE | |
| 40 | MGTRLLCWVVLGFLGTDHTGAGVSQSPRYKVAKRGQDVALRCDPISGHVSLFWYQQALGQGPEFLT | TCR D Vβ + |
| | YFQNEAQLDKSGLPSDRFFAERPEGSVSTLKIQRTQQEDSAVYLCASSLTAGAYNEQFFGPGTRLT | Cβ (aa) |
| | VLEDLKNVFPPEVAVFEPSEAEISHTQKATLVCLATGFYPDHVELSWWVNGKEVHSGVSTDPQPLK | |
| | EQPALNDSRYCLSSRLRVSATFWQNPRNHERCQVQFYGLSENDEWTQDRAKPVTQIVSAEAWGRAD | |
| | CGFTSESYQQGVLSATILYEILLGKATLYAVLVSALVLMAMVKRKDSRG | |
| 41 | MLLITSMLVLWMQLSQVNGQQVMQIPQYQHVQEGEDFTTYCNSSTTLSNIQWYKQRPGGHPVFLIQ | TCR E Vα |
| | LVKSGEVKKQKRLTFQFGEAKKNSSLHITATQTTDVGTYFCAADPRDNYGQNFVFGPGTRLSVLPY | (aa) |
| 42 | MLLITSMLVLWMQLSQVNGQQVMQIPQYQHVQEGEDFTTYCNSSTTLSNIQWYKQRPGGHPVFLIQ | TCR E Vα + |
| | LVKSGEVKKQKRLTFQFGEAKKNSSLHITATQTTDVGTYFCAADPRDNYGQNFVFGPGTRLSVLPY | Cα (aa) |
| | IQNPDPAVYQLRDSKSSDKSVCLFTDFDSQTNVSQSKDSDVYITDKTVLDMRSMDFKSNSAVAWSN | |
| | KSDFACANAFNNSIIPEDTFFPSPESSCDVKLVEKSFETDTNLNFQNLSVIGFRILLLKVAGENLL | |
| | MTLRLWSS | |
| 43 | MGTSLLCWMALCLLGADHADTGVSQNPRHKITKRGQNVTFRCDPISEHNRLYWYRQTLGQGPEFLT | TCR E Vβ |
| | YFQNEAQLEKSRLLSDRESAERPKGSFSTLEIQRTEQGDSAMYLCASSPLAGGETQYFGPGTRLLV | (aa) |
| | LE | |
| 44 | MGTSLLCWMALCLLGADHADTGVSQNPRHKITKRGQNVTFRCDPISEHNRLYWYRQTLGQGPEFLT | TCR E Vβ + |
| | YFQNEAQLEKSRLLSDRFSAERPKGSFSTLEIQRTEQGDSAMYLCASSPLAGGETQYFGPGTRLLV | Cβ (aa) |
| | LEDLKNVFPPEVAVFEPSEAEISHTQKATLVCLATGFYPDHVELSWWVNGKEVHSGVSTDPQPLKE | |
| | QPALNDSRYCLSSRLRVSATFWQNPRNHFRCQVQFYGLSENDEWTQDRAKPVTQIVSAEAWGRADC | |
| | GFTSESYQQGVLSATILYEILLGKATLYAVLVSALVLMAMVKRKDSRG | |
| 45 | METLLGLLILWLQLQWVSSKQEVTQIPAALSVPEGENLVLNCSFTDSAIYNLQWFRQDPGKGLTSL | TCR F Vα |
| | LLIQSSQREQTSGRLNASLDKSSGRSTLYIAASQPGDSATYLCAVRTKDYGGSQGNLIFGKGTKLS | (aa) |
| | VKPN | |
| 46 | METLLGLLILWLQLQWVSSKQEVTQIPAALSVPEGENLVLNCSFTDSAIYNLQWFRQDPGKGLTSL | TCR F Vα + |
| | LLIQSSQREQTSGRLNASLDKSSGRSTLYIAASQPGDSATYLCAVRTKDYGGSQGNLIFGKGTKLS | Cα (aa) |
| | VKPNIQNPDPAVYQLRDSKSSDKSVCLFTDFDSQTNVSQSKDSDVYITDKTVLDMRSMDFKSNSAV | |
| | AWSNKSDFACANAFNNSIIPEDTFFPSPESSCDVKLVEKSFETDTNLNFQNLSVIGFRILLLKVAG | |
| | FNLLMTLRLWSS | |
| 47 | MGTRLLCWVVLGFLGTDHTGAGVSQSPRYKVAKRGQDVALRCDPISGHVSLFWYQQALGQGPEFLT | TCR F Vβ |
| | YFQNEAQLDKSGLPSDRFFAERPEGSVSTLKIQRTQQEDSAVYLCASSLALPAADSYEQYFGPGTR | (aa) |
| | LTVTE | |
| 48 | MGTRLLCWVVLGFLGTDHTGAGVSQSPRYKVAKRGQDVALRCDPISGHVSLFWYQQALGQGPEFLT | TCR F Vβ + |
| | YFQNEAQLDKSGLPSDRFFAERPEGSVSTLKIQRTQQEDSAVYLCASSLALPAADSYEQYFGPGTR | Cβ (aa) |
| | LTVTEDLKNVFPPEVAVFEPSEAEISHTQKATLVCLATGFYPDHVELSWWVNGKEVHSGVSTDPQP | |
| | LKEQPALNDSRYCLSSRLRVSATFWQNPRNHFRCQVQFYGLSENDEWTQDRAKPVTQIVSAEAWGR | |
| | ADCGFTSESYQQGVLSATILYEILLGKATLYAVLVSALVLMAMVKRKDSRG | |
| 49 | MACPGFLWALVISTCLEFSMAQTVTQSQPEMSVQEAETVTLSCTYDTSESDYYLFWYKQPPSRQMI | TCR G Vα |
| | LVIRQEAYKQQNATENRFSVNFQKAAKSFSLKISDSQLGDAAMYFCAYRRFYNTDKLIFGTGTRLQ | (aa) |
| | VFPN | |
| 50 | MACPGFLWALVISTCLEFSMAQTVTQSQPEMSVQEAETVTLSCTYDTSESDYYLFWYKQPPSRQMI | TCR G Vα + |
| | LVIRQEAYKQQNATENRFSVNFQKAAKSFSLKISDSQLGDAAMYFCAYRRFYNTDKLIFGTGTRLQ | Cα (aa) |
| | VFPNIQNPDPAVYQLRDSKSSDKSVCLFTDFDSQTNVSQSKDSDVYITDKTVLDMRSMDFKSNSAV | |
| | AWSNKSDFACANAFNNSIIPEDTFFPSPESSCDVKLVEKSFETDTNLNFQNLSVIGFRILLLKVAG | |
| | FNLLMTLRLWSS | |
| 51 | MSNQVLCCVVLCFLGANTVDGGITQSPKYLFRKEGQNVTLSCEQNLNHDAMYWYRQDPGQGLRLIY | TCR G Vβ |
| | YSQIVNDFQKGDIAEGYSVSREKKESFPLTVTSAQKNPTAFYLCASRSAGYAEAFFGQGTRLTVVE | (aa) |
| 52 | MSNQVLCCVVLCFLGANTVDGGITQSPKYLFRKEGQNVTLSCEQNLNHDAMYWYRQDPGQGLRLIY | TCR G Vβ + |
| | YSQIVNDFQKGDIAEGYSVSREKKESFPLTVTSAQKNPTAFYLCASRSAGYAEAFFGQGTRLTVVE | Cβ (aa) |
| | DLNKVFPPEVAVFEPSEAEISHTQKATLVCLATGFFPDHVELSWWVNGKEVHSGVSTDPQPLKEQP | |
| | ALNDSRYCLSSRLRVSATFWQNPRNHFRCQVQFYGLSENDEWTQDRAKPVTQIVSAEAWGRADCGF | |
| | TSVSYQQGVLSATILYEILLGKATLYAVLVSALVLMAMVKRKDF | |
| 53 | METLLGLLILWLQLQWVSSKQEVTQIPAALSVPEGENLVLNCSFTDSAIYNLQWFRQDPGKGLTSL | TCR H Vα |
| | LLIQSSQREQTSGRLNASLDKSSGRSTLYIAASQPGDSATYLCAVRSGYGGSQGNLIFGKGTKLSV | (aa) |
| | KPN | |
| 54 | METLLGLLILWLQLQWVSSKQEVTQIPAALSVPEGENLVLNCSFTDSAIYNLQWFRQDPGKGLTSL | TCR H Vα + |
| | LLIQSSQREQTSGRLNASLDKSSGRSTLYIAASQPGDSATYLCAVRSGYGGSQGNLIFGKGTKLSV | Cα (aa) |
| | KPNIQNPDPAVYQLRDSKSSDKSVCLFTDFDSQTNVSQSKDSDVYITDKTVLDMRSMDFKSNSAVA | |
| | WSNKSDFACANAFNNSIIPEDTFFPSPESSCDVKLVEKSFETDTNLNFQNLSVIGFRILLLKVAGF | |
| | NLLMTLRLWSS | |
| 55 | MGTRLLCWVVLGFLGTDHTGAGVSQSPRYKVAKRGQDVALRCDPISGHVSLFWYQQALGQGPEFLT | TCR H Vβ |
| | YFQNEAQLDKSGLPSDRFFAERPEGSVSTLKIQRTQQEDSAVYLCASSLAGGSLQETQYFGPGTRL | (aa) |
| | LVLE | |
| 56 | MGTRLLCWVVLGFLGTDHTGAGVSQSPRYKVAKRGQDVALRCDPISGHVSLFWYQQALGQGPEFLT | TCR H Vβ + |
| | YFQNEAQLDKSGLPSDRFFAERPEGSVSTLKIQRTQQEDSAVYLCASSLAGGSLQETQYFGPGTRL | Cβ (aa) |
| | LVLEDLKNVFPPEVAVFEPSEAEISHTQKATLVCLATGFYPDHVELSWWVNGKEVHSGVSTDPQPL | |
| | KEQPALNDSRYCLSSRLRVSATFWQNPRNHERCQVQFYGLSENDEWTQDRAKPVTQIVSAEAWGRA | |
| | DCGFTSESYQQGVLSATILYEILLGKATLYAVLVSALVLMAMVKRKDSRG | |
| 57 | METLLGLLILWLQLQWVSSKQEVTQIPAALSVPEGENLVLNCSFTDSAIYNLQWFRQDPGKGLTSL | TCR I Vα |
| | LLIQSSQREQTSGRLNASLDKSSGRSTLYIAASQPGDSATYLCAVRPKNYGGSQGNLIFGKGTKLS | (aa) |
| | VKPN | |
| 58 | METLLGLLILWLQLQWVSSKQEVTQIPAALSVPEGENLVLNCSFTDSAIYNLQWFRQDPGKGLTSL | TCR I Vα + |
| | LLIQSSQREQTSGRLNASLDKSSGRSTLYIAASQPGDSATYLCAVRPKNYGGSQGNLIFGKGTKLS | Cα (aa) |
| | VKPNIQNPDPAVYQLRDSKSSDKSVCLFTDFDSQTNVSQSKDSDVYITDKTVLDMRSMDFKSNSAV | |
| | AWSNKSDFACANAFNNSIIPEDTFFPSPESSCDVKLVEKSFETDTNLNFQNLSVIGFRILLLKVAG | |
| | FNLLMTLRLWSS | |
| 59 | MGTRLLCWVVLGFLGTDHTGAGVSQSPRYKVAKRGQDVALRCDPISGHVSLFWYQQALGQGPEFLT | TCR I Vβ |
| | YFQNEAQLDKSGLPSDRFFAERPEGSVSTLKIQRTQQEDSAVYLCASINPRKGGYTFGSGTRLTVV | (aa) |
| | E | |
| 60 | MGTRLLCWVVLGFLGTDHTGAGVSQSPRYKVAKRGQDVALRCDPISGHVSLFWYQQALGQGPEFLT | TCR I Vβ + |
| | YFQNEAQLDKSGLPSDRFFAERPEGSVSTLKIQRTQQEDSAVYLCASINPRKGGYTFGSGTRLTVV | Cβ (aa) |
| | EDLNKVFPPEVAVFEPSEAEISHTQKATLVCLATGFFPDHVELSWWVNGKEVHSGVSTDPQPLKEQ | |
| | PALNDSRYCLSSRLRVSATFWQNPRNHFRCQVQFYGLSENDEWTQDRAKPVTQIVSAEAWGRADCG | |
| | FTSVSYQQGVLSATILYEILLGKATLYAVLVSALVLMAMVKRKDF | |
| 61 | METLLGLLILWLQLQWVSSKQEVTQIPAALSVPEGENLVLNCSFTDSAIYNLQWFRQDPGKGLTSL | TCR J Vα |
| | LLIQSSQREQTSGRLNASLDKSSGRSTLYIAASQPGDSATYLCAVRPGNYGGSQGNLIFGKGTKLS | (aa) |
| | VKPN | |
| 62 | METLLGLLILWLQLQWVSSKQEVTQIPAALSVPEGENLVLNCSFTDSAIYNLQWFRQDPGKGLTSL | TCR J Vα + |
| | LLIQSSQREQTSGRLNASLDKSSGRSTLYIAASQPGDSATYLCAVRPGNYGGSQGNLIFGKGTKLS | Cα (aa) |
| | VKPNIQNPDPAVYQLRDSKSSDKSVCLFTDFDSQTNVSQSKDSDVYITDKTVLDMRSMDFKSNSAV | |
| | AWSNKSDFACANAFNNSIIPEDTFFPSPESSCDVKLVEKSFETDTNLNFQNLSVIGFRILLLKVAG | |
| | FNLLMTLRLWSS | |
| 63 | MGTRLLCWVVLGFLGTDHTGAGVSQSPRYKVAKRGQDVALRCDPISGHVSLFWYQQALGQGPEFLT | TCR J Vβ |
| | YFQNEAQLDKSGLPSDRFFAERPEGSVSTLKIQRTQQEDSAVYLCASSPPGGLGEKLFFGSGTQLS | (aa) |
| | VLE | |
| 64 | MGTRLLCWVVLGFLGTDHTGAGVSQSPRYKVAKRGQDVALRCDPISGHVSLFWYQQALGQGPEFLT | TCR J Vβ + |
| | YFQNEAQLDKSGLPSDRFFAERPEGSVSTLKIQRTQQEDSAVYLCASSPPGGLGEKLFFGSGTQLS | Cβ (aa) |
| | VLEDLNKVFPPEVAVFEPSEAEISHTQKATLVCLATGFFPDHVELSWWVNGKEVHSGVSTDPQPLK | |
| | EQPALNDSRYCLSSRLRVSATFWQNPRNHFRCQVQFYGLSENDEWTQDRAKPVTQIVSAEAWGRAD | |
| | CGFTSVSYQQGVLSATILYEILLGKATLYAVLVSALVLMAMVKRKDF | |
| 65 | METLLGLLILWLQLQWVSSKQEVTQIPAALSVPEGENLVLNCSFTDSAIYNLQWFRQDPGKGLTSL | TCR K Vα |
| | LLIQSSQREQTSGRLNASLDKSSGRSTLYIAASQPGDSATYLCAVRPVNYGGSQGNLIFGKGTKLS | (aa) |
| | VKPN | |
| 66 | METLLGLLILWLQLQWVSSKQEVTQIPAALSVPEGENLVLNCSFTDSAIYNLQWFRQDPGKGLTSL | TCR K Vα + |
| | LLIQSSQREQTSGRLNASLDKSSGRSTLYIAASQPGDSATYLCAVRPVNYGGSQGNLIFGKGTKLS | Cα (aa) |
| | VKPNIQNPDPAVYQLRDSKSSDKSVCLFTDFDSQTNVSQSKDSDVYITDKTVLDMRSMDFKSNSAV | |
| | AWSNKSDFACANAFNNSIIPEDTFFPSPESSCDVKLVEKSFETDTNLNFQNLSVIGFRILLLKVAG | |
| | FNLLMTLRLWSS | |
| 67 | MGTRLLCWVVLGFLGTDHTGAGVSQSPRYKVAKRGQDVALRCDPISGHVSLFWYQQALGQGPEFLT | TCR K Vβ |
| | YFQNEAQLDKSGLPSDRFFAERPEGSVSTLKIQRTQQEDSAVYLCASRSPRLETQYFGPGTRLLVL | (aa) |
| | E | |
| 68 | MGTRLLCWVVLGFLGTDHTGAGVSQSPRYKVAKRGQDVALRCDPISGHVSLFWYQQALGQGPEFLT | TCR K Vβ + |
| | YFQNEAQLDKSGLPSDRFFAERPEGSVSTLKIQRTQQEDSAVYLCASRSPRLETQYFGPGTRLLVL | Cβ (aa) |
| | EDLKNVFPPEVAVFEPSEAEISHTQKATLVCLATGFYPDHVELSWWVNGKEVHSGVSTDPQPLKEQ | |
| | PALNDSRYCLSSRLRVSATFWQNPRNHFRCQVQFYGLSENDEWTQDRAKPVTQIVSAEAWGRADCG | |
| | FTSESYQQGVLSATILYEILLGKATLYAVLVSALVLMAMVKRKDSRG | |
| 69 | METLLGLLILWLQLQWVSSKQEVTQIPAALSVPEGENLVLNCSFTDSAIYNLQWFRQDPGKGLTSL | TCR L Vα |
| | LLIQSSQREQTSGRLNASLDKSSGRSTLYIAASQPGDSATYLCAVRTKDYGGSQGNLIFGKGTKLS | (aa) |
| | VKPN | |
| 70 | METLLGLLILWLQLQWVSSKQEVTQIPAALSVPEGENLVLNCSFTDSAIYNLQWFRQDPGKGLTSL | TCR L Vα + |
| | LLIQSSQREQTSGRLNASLDKSSGRSTLYIAASQPGDSATYLCAVRTKDYGGSQGNLIFGKGTKLS | Cα (aa) |
| | VKPNIQNPDPAVYQLRDSKSSDKSVCLFTDFDSQTNVSQSKDSDVYITDKTVLDMRSMDFKSNSAV | |
| | AWSNKSDFACANAFNNSIIPEDTFFPSPESSCDVKLVEKSFETDTNLNFQNLSVIGFRILLLKVAG | |
| | FNLLMTLRLWSS | |
| 71 | MGTRLLCWVVLGFLGTDHTGAGVSQSPRYKVAKRGQDVALRCDPISGHVSLFWYQQALGQGPEFLT | TCR L Vβ |
| | YFQNEAQLDKSGLPSDRFFAERPEGSVSTLKIQRTQQEDSAVYLCASSFPGLVSYEQYFGPGTRLT | (aa) |
| | VTE | |
| 72 | MGTRLLCWVVLGFLGTDHTGAGVSQSPRYKVAKRGQDVALRCDPISGHVSLFWYQQALGQGPEFLT | TCR L Vβ + |
| | YFQNEAQLDKSGLPSDRFFAERPEGSVSTLKIQRTQQEDSAVYLCASSFPGLVSYEQYFGPGTRLT | Cβ (aa) |
| | VTEDLKNVFPPEVAVFEPSEAEISHTQKATLVCLATGFYPDHVELSWWVNGKEVHSGVSTDPQPLK | |
| | EQPALNDSRYCLSSRLRVSATFWQNPRNHERCQVQFYGLSENDEWTQDRAKPVTQIVSAEAWGRAD | |
| | CGFTSESYQQGVLSATILYEILLGKATLYAVLVSALVLMAMVKRKDSRG | |
| 73 | METLLGLLILWLQLQWVSSKQEVTQIPAALSVPEGENLVLNCSFTDSAIYNLQWFRQDPGKGLTSL | TCR M Vα |
| | LLIQSSQREQTSGRLNASLDKSSGRSTLYIAASQPGDSATYLCAVRSGYGGSQGNLIFGKGTKLSV | (aa) |
| | KPN | |
| 74 | METLLGLLILWLQLQWVSSKQEVTQIPAALSVPEGENLVLNCSFTDSAIYNLQWFRQDPGKGLTSL | TCR M Vα + |
| | LLIQSSQREQTSGRLNASLDKSSGRSTLYIAASQPGDSATYLCAVRSGYGGSQGNLIFGKGTKLSV | Cα (aa) |
| | KPNIQNPDPAVYQLRDSKSSDKSVCLFTDFDSQTNVSQSKDSDVYITDKTVLDMRSMDFKSNSAVA | |
| | WSNKSDFACANAFNNSIIPEDTFFPSPESSCDVKLVEKSFETDTNLNFQNLSVIGFRILLLKVAGE | |
| | NLLMTLRLWSS | |
| 75 | MGTRLLCWVVLGFLGTDHTGAGVSQSPRYKVAKRGQDVALRCDPISGHVSLFWYQQALGQGPEFLT | TCR M Vβ |
| | YFQNEAQLDKSGLPSDRFFAERPEGSVSTLKIQRTQQEDSAVYLCASSLAGQETQYFGPGTRLLVL | (aa) |
| | E | |
| 76 | MGTRLLCWVVLGFLGTDHTGAGVSQSPRYKVAKRGQDVALRCDPISGHVSLFWYQQALGQGPEFLT | TCR M Vβ + |
| | YFQNEAQLDKSGLPSDRFFAERPEGSVSTLKIQRTQQEDSAVYLCASSLAGQETQYFGPGTRLLVL | Cβ (aa) |
| | EDLKNVFPPEVAVFEPSEAEISHTQKATLVCLATGFYPDHVELSWWVNGKEVHSGVSTDPQPLKEQ | |
| | PALNDSRYCLSSRLRVSATFWQNPRNHFRCQVQFYGLSENDEWTQDRAKPVTQIVSAEAWGRADCG | |
| | FTSESYQQGVLSATILYEILLGKATLYAVLVSALVLMAMVKRKDSRG | |
| 77 | METLLGLLILWLQLQWVSSKQEVTQIPAALSVPEGENLVLNCSFTDSAIYNLQWFRQDPGKGLTSL | TCR N Vα |
| | LLIQSSQREQTSGRLNASLDKSSGRSTLYIAASQPGDSATYLCAVRQGYGGSQGNLIFGKGTKLSV | (aa) |
| | KPN | |
| 78 | METLLGLLILWLQLQWVSSKQEVTQIPAALSVPEGENLVLNCSFTDSAIYNLQWFRQDPGKGLTSL | TCR N Vα + |
| | LLIQSSQREQTSGRLNASLDKSSGRSTLYIAASQPGDSATYLCAVRQGYGGSQGNLIFGKGTKLSV | Cα (aa) |
| | KPNIQNPDPAVYQLRDSKSSDKSVCLFTDFDSQTNVSQSKDSDVYITDKTVLDMRSMDFKSNSAVA | |
| | WSNKSDFACANAFNNSIIPEDTFFPSPESSCDVKLVEKSFETDTNLNFQNLSVIGFRILLLKVAGF | |
| | NLLMTLRLWSS | |
| 79 | MGTRLLCWVVLGFLGTDHTGAGVSQSPRYKVAKRGQDVALRCDPISGHVSLFWYQQALGQGPEFLT | TCR N Vβ |
| | YFQNEAQLDKSGLPSDRFFAERPEGSVSTLKIQRTQQEDSAVYLCASSLIQGTNSPLHFGNGTRLT | (aa) |
| | VTE | |
| 80 | MGTRLLCWVVLGFLGTDHTGAGVSQSPRYKVAKRGQDVALRCDPISGHVSLFWYQQALGQGPEFLT | TCR N Vβ + |
| | YFQNEAQLDKSGLPSDRFFAERPEGSVSTLKIQRTQQEDSAVYLCASSLIQGTNSPLHFGNGTRLT | Cβ (aa) |
| | VTEDLNKVFPPEVAVFEPSEAEISHTQKATLVCLATGFFPDHVELSWWVNGKEVHSGVSTDPQPLK | |
| | EQPALNDSRYCLSSRLRVSATFWQNPRNHERCQVQFYGLSENDEWTQDRAKPVTQIVSAEAWGRAD | |
| | CGFTSVSYQQGVLSATILYEILLGKATLYAVLVSALVLMAMVKRKDF | |
| 81 | METLLGLLILWLQLQWVSSKQEVTQIPAALSVPEGENLVLNCSFTDSAIYNLQWFRQDPGKGLTSL | TCR O Vα |
| | LLIQSSQREQTSGRLNASLDKSSGRSTLYIAASQPGDSATYLCAVRPKNYGGSQGNLIFGKGTKLS | (aa) |
| | VKPN | |
| 82 | METLLGLLILWLQLQWVSSKQEVTQIPAALSVPEGENLVLNCSFTDSAIYNLQWFRQDPGKGLTSL | TCR O Vα + |
| | LLIQSSQREQTSGRLNASLDKSSGRSTLYIAASQPGDSATYLCAVRPKNYGGSQGNLIFGKGTKLS | Cα (aa) |
| | VKPNIQNPDPAVYQLRDSKSSDKSVCLFTDFDSQTNVSQSKDSDVYITDKTVLDMRSMDFKSNSAV | |
| | AWSNKSDFACANAFNNSIIPEDTFFPSPESSCDVKLVEKSFETDTNLNFQNLSVIGFRILLLKVAG | |
| | FNLLMTLRLWSS | |
| 83 | MGTRLLCWVVLGFLGTDHTGAGVSQSPRYKVAKRGQDVALRCDPISGHVSLFWYQQALGQGPEFLT | TCR O Vβ |
| | YFQNEAQLDKSGLPSDRFFAERPEGSVSTLKIQRTQQEDSAVYLCASSLAERPGTQYFGPGTRLTV | (aa) |
| | LE | |
| 84 | MGTRLLCWVVLGFLGTDHTGAGVSQSPRYKVAKRGQDVALRCDPISGHVSLFWYQQALGQGPEFLT | TCR O Vβ + |
| | YFQNEAQLDKSGLPSDRFFAERPEGSVSTLKIQRTQQEDSAVYLCASSLAERPGTQYFGPGTRLTV | Cβ (aa) |
| | LEDLKNVFPPEVAVFEPSEAEISHTQKATLVCLATGFYPDHVELSWWVNGKEVHSGVSTDPQPLKE | |
| | QPALNDSRYCLSSRLRVSATFWQNPRNHFRCQVQFYGLSENDEWTQDRAKPVTQIVSAEAWGRADC | |
| | GFTSESYQQGVLSATILYEILLGKATLYAVLVSALVLMAMVKRKDSRG | |
| 85 | METLLGLLILWLQLQWVSSKQEVTQIPAALSVPEGENLVLNCSFTDSAIYNLQWFRQDPGKGLTSL | TCR P Vα |
| | LLIQSSQREQTSGRLNASLDKSSGRSTLYIAASQPGDSATYLCAVRPKNYGGSQGNLIFGKGTKLS | (aa) |
| | VKPN | |
| 86 | METLLGLLILWLQLQWVSSKQEVTQIPAALSVPEGENLVLNCSFTDSAIYNLQWFRQDPGKGLTSL | TCR P Vα + |
| | LLIQSSQREQTSGRLNASLDKSSGRSTLYIAASQPGDSATYLCAVRPKNYGGSQGNLIFGKGTKLS | Cα (aa) |
| | VKPNIQNPDPAVYQLRDSKSSDKSVCLFTDFDSQTNVSQSKDSDVYITDKTVLDMRSMDFKSNSAV | |
| | AWSNKSDFACANAFNNSIIPEDTFFPSPESSCDVKLVEKSFETDTNLNFQNLSVIGFRILLLKVAG | |
| | FNLLMTLRLWSS | |
| 87 | MGTRLLCWVVLGFLGTDHTGAGVSQSPRYKVAKRGQDVALRCDPISGHVSLFWYQQALGQGPEFLT | TCR P Vβ |
| | YFQNEAQLDKSGLPSDRFFAERPEGSVSTLKIQRTQQEDSAVYLCASSLIQGTNSPLHEGNGTRLT | (aa) |
| | VTE | |
| 88 | MGTRLLCWVVLGFLGTDHTGAGVSQSPRYKVAKRGQDVALRCDPISGHVSLFWYQQALGQGPEFLT | TCR P Vβ + |
| | YFQNEAQLDKSGLPSDRFFAERPEGSVSTLKIQRTQQEDSAVYLCASSLIQGTNSPLHFGNGTRLT | Cβ (aa) |
| | VTEDLNKVFPPEVAVFEPSEAEISHTQKATLVCLATGFFPDHVELSWWVNGKEVHSGVSTDPQPLK | |
| | EQPALNDSRYCLSSRLRVSATFWQNPRNHFRCQVQFYGLSENDEWTQDRAKPVTQIVSAEAWGRAD | |
| | CGFTSVSYQQGVLSATILYEILLGKATLYAVLVSALVLMAMVKRKDF | |

### Examples

### Example 1. Manufacture of Engineered T Cells

Engineered T cells are manufactured from an allogeneic hematopoietic stem cell transplant donor's peripheral blood obtained via an apheresis procedure. T cells are enriched via CD8+ selection and placed in culture with cytokines. The cultured cells are activated (e.g., with activating anti-CD3 and anti CD28 antibodies) in combination with support reagents. Following activation, the activated CD8⁺ T cells are electroporated to deliver CRISPR/Cas9 RNP guide in order to knockout the endogenous TCRs of the cultured cells. The resultant cell population is returned to culture and genetically modified ex vivo using a lentiviral vector encoding the heterologous TCR (e.g., a TCR targeting tumor epitope; e.g., a hematopoietic restricted miHA epitope; e.g., the miHA HA-1 “H” epitope). The transduced cell population is then expanded, harvested, formulated, filled, finished and optionally cryopreserved. The engineered T cells are an allogeneic immune-cellular therapy that utilizes an HLA-matched donor's cells as the starting material. The manufacturing process is designed to generate a high purity T cell population were the endogenous TCR expression is ablated through gene editing and replaced through lentivirus vector transduction to enable expression of a heterologous TCR. An exemplary manufacturing process is summarized below:
1. An apheresis product or PBMCs are collected, by apheresis (without prior mobilization), from a donor subject HLA-matched to the recipient subject and not expressing an epitope expressed in the recipient subject.
2. CD8⁺ cells are selected from the apheresis product or PBMCs to form an enriched CD8⁺ T cell population.
3. The enriched CD8⁺ T cells are placed in culture and activated. Methods of activation include, but are not limited to, contacting the CD8⁺ T cells with immobilized recombinant CD3 and CD28 agonists. Immobilized recombinant CD3 and CD28 agonists include, but are not limited to, MACS® GMP T Cell TransAct™ (Miltenyi Biotec).
4. The endogenous TCR expression is ablated through a multiplex gene editing of the TRAC and TRBC genes. Electroporation is used to introduce a ribonucleoprotein comprising Spyfi SpCas9 and sgRNA into the activated T cells to knockout the endogenous TCR in the activated CD8⁺ donor T cells to form edited T cells.
5. Transduction of the edited T cells with a self-inactivating minimal lentivirus vector introduces the expression cassette for heterologous TCR or CAR, optionally with a marker to form the engineered T cells. The marker can be, but is not limited to, RQR8.
6. The engineered T cells are expanded ex vivo by contacting the engineered T cells with cytokines and incubating in conditions suitable for growth and proliferation of the cells. The cytokines include, but are not limited to, IL-2, optionally with one or more of IL-7, IL-15, IL-21, IL-9.
7. The expanded engineered T cells are washed, placed into contained (e.g., infusion bags) and optionally cryopreserved.

### Example 2. Affect of Activation Method on Manufacture of Engineered T Cells Expressing a Heterologous TCR

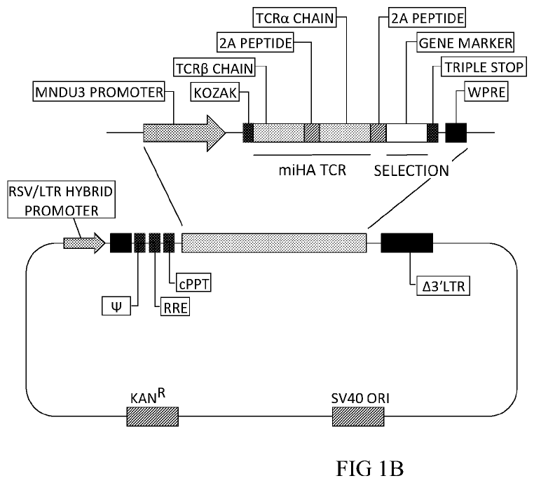

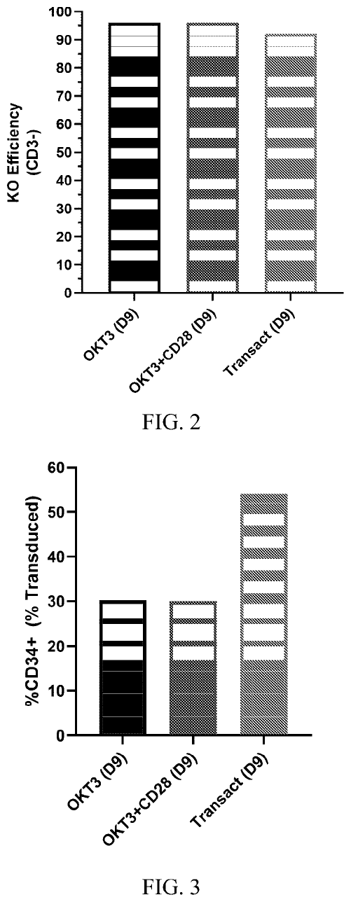

PBMCs were collected from a donor subject, activated with OKT3 (anti-CD3 monoclonal antibodies), OKT3 anti-CD28 antibodies, or TRANSACT™ (beads containing CD3 and CD28 agonists), and CD8⁺ cells were selected. Alternatively, CD8+ cells were purified from PBMCs collected from a donor subject, and activated with OKT3 (anti-CD3 monoclonal antibodies), OKT3 and anti-CD28 antibodies, or TRANSACT™ (beads containing CD3 and CD28 agonists). Following activation and selection (or selection and activation), cells were processed for knockout of the endogenous TCR by CRISPR and transduced with a lentivirus encoding a heterologous TCR. Activation was performed on day 0. CD8⁺ cells were selected, processed for knockout of the endogenous TCR and transduced with lentivirus on day 1. Cells are analyzed for marker expression, endogenous TCR knockout, and heterologous TCR expression on day 4. As shown in Table 4, activation of PBMCs with each of the methods resulted in greater than 90% of cells being CD8⁺ cells following activation. Also as shown in Table 4, 64-73% of cells were CD8⁺CD25⁺ and greater than 98% of cells were CD8⁺CD69⁺ at day 4 for each of the activation methods. As shown in FIG. 2, knockout of the endogenous TCR was highly efficient (>90%) in cells produced by each of the activation methods. Greater than 90% of cells were also found to be CD3⁻ as determined by flow cytometry after CRISPR knockout of the endogenous TCR by before transduction of the heterologous TCR. The method of activation of PBMCs did appear to have a significant effect of the percentage of cells that expressed the heterologous HA-1 TCR following transduction with lentivirus encoding the heterologous TCR (FIG. 3). However, even with CD3 and CD3/CD28 activation, greater than 30% of cells expressed the heterologous HA-1 TCR.

TABLE 4
Analysis of cell marker expression following activation of T cells.

| | percent | percent | percent |
|---|---|---|---|
| | CD8⁺ | CD8⁺CD25⁺ | CD8⁺CD69⁺ |
| Activator | cells | cells | cells |
| OKT3 | 99.4 | 65.4 | 98.7 |
| OKT3/CD28 | 97.7 | 73.0 | 98.4 |
| Transact | 93.3 | 64.2 | 98.6 |

### Example 3. Effect of Activation Method of Cytolytic Potency of Engineered T Cells

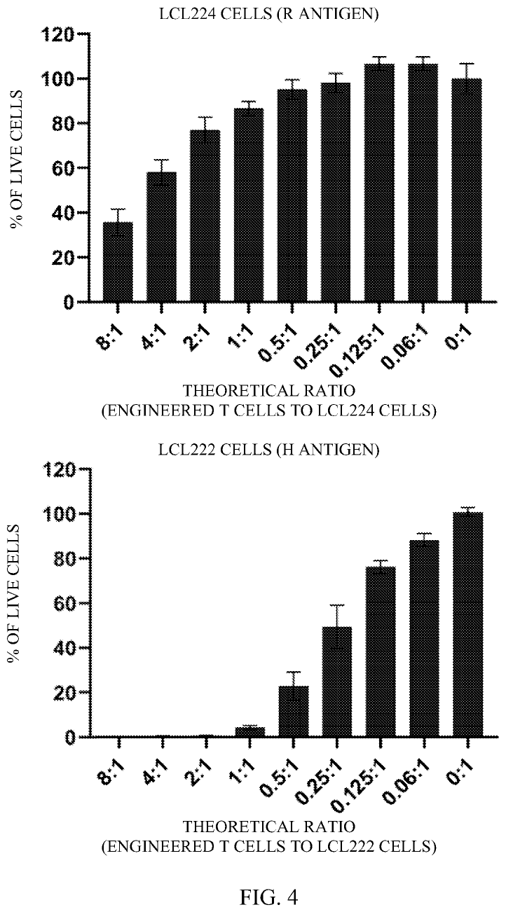

Engineered T cells produced from PBMCs activated as described above were analyzed for cytolytic potency. Cytolytic potency was tested against LCL222 cells (expressing the target MiHA HA-1 H antigen (H/H cells)) or against control LCL224 cells (expressing the non-target MiHA HA-1 R antigen (R/R cells)) As shown in FIG. 4, efficient killing was observed for the engineered T cells against LCL222 cells expressing the HA-1 H antigen but not against LCL224 cells expressing the HA-1 R antigen. Efficiency killing was also observed for THP cells expressing the HA-1 H antigen. Engineered T cells formed using TRANSACT™ to activate the donor PBMCs were more potent than engineered T cells formed using CD3 or CD3 plus CD28 activation. Engineered T cells formed using TRANSACT™ to activate enriched CD8⁺ cells were also more potent compared to engineered T cells formed from activation of PBMCs prior to CD8⁺ enrichment (i.e., unpurified PBMC donor cells).

TABLE 5
Cell killing assay IC₅₀ results

| | IC₅₀ | | |
|---|---|---|---|
| | LCL222 | THP-1 | THP-1 |
| | cells | Luc cells | cells |
| ARM1 | | 0.08896 | 0.1129 |
| ARM2 | 0.08209 | | |
| ARM3 | | 0.1345 | 0.08910 |
| ARM4 | 0.1171 | 0.01551 | 0.009738 |
| ARM5 | 0.08223 | | |
| ARM7 | 0.08502 | | |
| ARM8A | 0.1316 | | |

### Example 4. Functional Activity of Engineered T Cells

The engineered T cells are designed to kill target cells (e.g., a patient's leukemic cells). This is possible by matching a healthy donor subject with an appropriate recipient subject. Engineered T cells were made using T cells from a donor subject that was HLA-A*02:01⁺/HA-1 R/R for use in a recipient subject that was HLA-A*02:01⁺/HA-1 H/R or H/H. The engineered T cells were modified to express an anti-HA-1 “H” epitope specific TCR. Flow cytometry was used to assess CD69 expression. CD69 expression is rapidly induced on the surface of T lymphocytes after TCR/CD3 engagement, activating cytokines and polyclonal, mitogenic stimulation. Cytotoxicity and cytokine release assays were used to assess engineered T cell function.

Engineered T cells: Cryopreserved healthy donor derived PBMCs collected from a donor were activated with a soluble anti-CD3 antibody and cultured again overnight. The next day, the PBMCs were split to allow some to be used as PBMCs and the rest were enriched for CD8⁺ T cells. Both the PBMCs and the CD8+ cells were then nucleofected and transduced and placed into culture to expand. Engineered T cells were then collected on either day 6 or 11. Cells were assayed for % CD34⁺ and CD3⁺ cells as well as % cells that bound to an HA-1 dextramer. The remaining cells were then used for the cytotoxicity assay and cytokine release.

Target cells: LCLs, either naturally expressing HA-1 VLHDDLLEA peptide (“H” peptide, H/H) (referred to as LCL222 cells) or loaded with the immunogenic or the non-immunogenic HA-1 VLRDDLLEA peptide (“R” peptide R/R) (referred to as LCL224 cells) were used.

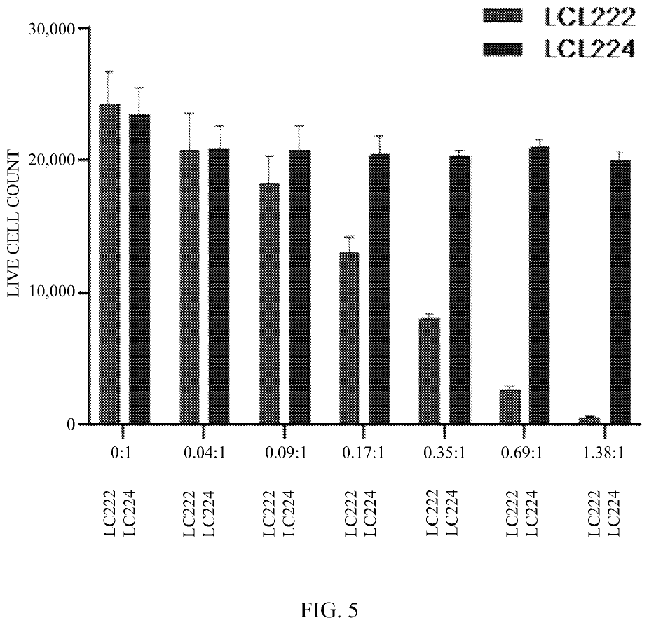

Cytotoxicity killing Assay: LCL222 cells and LCL224 cells were differentially stained with 0.5 μM CFDA-SE (5(6)-carboxyfluorescein diacetate succinimidyl ester, STEMCELL™ Technologies) (LCL222, “High”) and 0.025 μM CFDA-SE (LCL224, “Low”) so they could be distinguished. LCL and engineered T cells were combined at the ratios indicated in FIG. 5. Each assay contained 100,000 LCL cells (50,000 LCL222 cells and 50,000 LCL224 cells). Cells were combined in culture media in 96-well U-bottom plates and cultured overnight (16-18 hours) in an incubator at 37° C. and 5% CO₂. Specific killing removes LCL222 cells while not affecting LCL224 cells. Samples were run in triplicate and analyzed for cell counts by flow cytometry and gating on the CFSE populations. Specific killing was detected by the loss of LCL cells loaded with the immunogenic “H” peptide but not LCLs loaded with “R” peptide. The data shown in FIG. 5 clearly show the specificity of the engineered T cells in killing target cells expression the HA-1 “H” epitope (LC222 cells) The number of LCL222 cells dropped to almost undetectable levels at a E:T ratio of 1.38:1, while the number of LCL224 cells remained relatively unchanged. Using this data, an EC50 value of 0.49:1 was calculated.

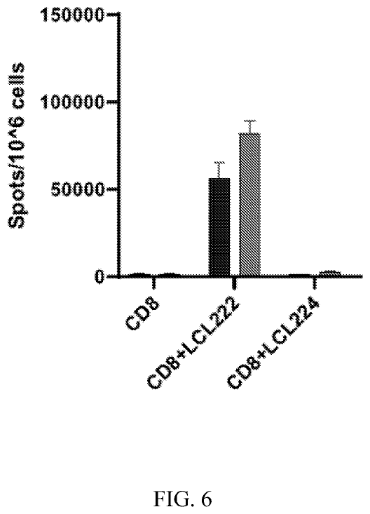

IL-2 ELISpot Cytokine Release Assay: Activated CD8⁺ T cell secrete cytokines (e.g., IL-2, TNF-α, IFN-γ), perforin or granzyme. Such proteins have anti-tumor effects. An enzyme-linked immune absorbent spot (ELISpot) was used to detect the frequency of cytokine secretion from engineered T cells. Engineered T cells and target cells were seeded on a PVDF or nitrocellulose membrane in a 96-well plate precoated with an antibody specific to the secreted cytokine. Secreted cytokines were captured and further detected using biotinylated antibody. Engineered T cells were mixed at a 1:1 ratio with LCL222 or LCL224 target cells and incubated for 16±2 hours at 37° C. and 5% CO₂. In some samples, LCL224 cells (R/R) were also loaded with the HA-1 “H” peptide. ELISpot analysis of IL-2 secretion was performed following the manufacturer's suggested protocol (Human IL-2 ELISpotbasic, MabTech). Engineered T cells alone and target cells alone were used as negative controls. Engineered T cells activated with PMA (50 ng/mL) and Ionomycin (1 μM) were used as a positive control. The sensitivity of ELISpot allows the detection of low-frequency antigen-specific T-cells that secrete cytokines and effector molecules. Data for IL-2 from two independent experiments using PBMCs from different donors are shown in FIG. 6. High IL-2 secretion was observed upon activation with cells that present the HA-1 “H” peptide (LCL222), but not the HA-1 “R” peptide (LCL224).

Intracellular Cytokine Assay: LCL222 and LCL224 target cells were labeled for 30 minutes with 0.1 μM of CellTracker Red CMTPX before use. Engineered T cells were cocultured with either the LCL222 or LCL224 at a ratio of 1:1 for 6 hours in the presence of Golgi and endosome inhibitors to allow intracellular accumulation of cytokines and other proteins. Engineered T cells alone served as a negative control. Engineered T cells activated with Phorbol 12-myristate 13-acetate (PMA, 50 ng/mL) and Ionomycin (1 μM) served as a positive control. After the incubation, the cells were collected and washed by centrifugation. The cells were then stained with LIVE/DEAD Ghost 510, FITC-conjugated anti-CD8, and AF700-conjugated anti-CD34. After fixation and permeabilization, cells were stained with APC-conjugated anti-IL2, Brilliant Violet (BV) 421-conjugated INFγ, phycoerythrin (PE)-cyanine 7-conjugated anti-GranzymeB, and phycoerythrin(PE)-Cy5 Conjugated anti-CD107a. Cells were acquired and analyzed via ACEA NovoCyte Quanteon flow cytometer by the following gating strategy: Forward Scatter vs. Side Scatter for lymphocytes, singlets, live cells by Ghost 510, CellTracker Red CMTPX (PE channel) negative population, CD8+ cells, CD34+ cells, INFγ+ cells, IL2+ cells, CD107a+ cells and Granzyme B-positive cells.

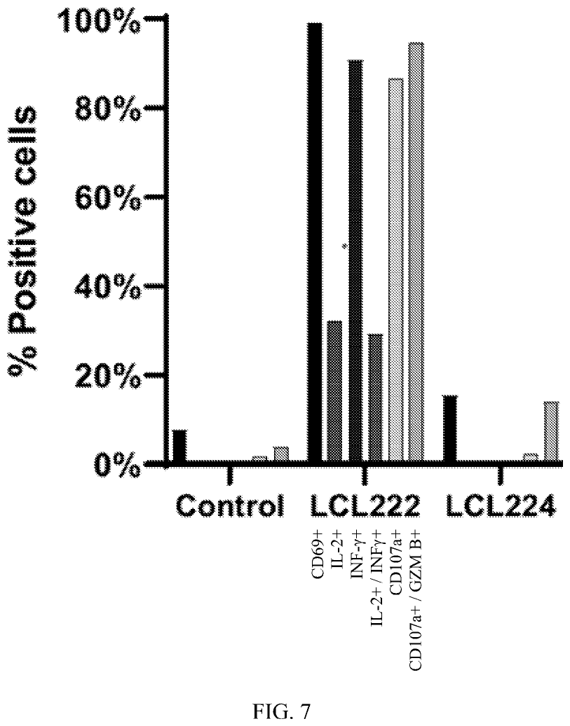

Flow cytometry was used to analyze cytokine expression as well as CD107a and granzyme B (GZM B) expression. As shown in FIG. 7, in the presence of LCL222 cells, (expressing the “H” peptide), but not LCL224 cells (expressing the “H” peptide), engineered T cells expressing a heterologous anti-HA-1 “H” epitope TCR become activated and show increased staining for CD69, IL-2, INF-7, CD107a, and GZM B.

### Example 5. Alloreactivity Assay

The HA-1 peptide epitope is presented on the cell surface of malignant hematopoietic cells in association with HLA-A*02:01 or HLA-A*02:06. To show that the engineered T cells expressing an anti-HA-1 TCR are not reactive against HLA types other than HLA-A*02:01 or HLA-A*02:06, a panel of LCL cells that present the restricting HLA-A*02:01, HLA-A*02:06 as well as other HLA alleles, and have been genotyped for HA-1 haplotypes (R/R, H/R or H/H), were cocultured with engineered T cells expressing a heterologous anti-HA-1 “H” epitope TCR.

Engineered T cells expressing a heterologous anti-HA-1 “H” epitope TCR were prepared as described.

EBV-immortalized B cells, lymphoblastoid cell lines (LCLs), were collected from commercial sources and screened for HLA-A and HA−1 genotype status. LCL cells expressing various HLA alleles and HA-1 “H” epitope or HA-1 “R” epitope were identified. The LCL cells were labeled with loaded with CellTracker Red CMTPX to allow separation of the target cells by flow cytometry. Engineered T cells were cocultured with the LCLs at a ratio of 0.5-0.7:1 (TCR-expressing T cells to target cells) for 4 hours. Cells were collected and stained with the live/dead violet 510 Ghost dye, FITC-anti-CD8, AF700-anti-CD34 and APC-anti-CD69 antibodies. A control sample (CC) of the engineered T cells alone was included for background staining. Cells were acquired and analyzed with an ACEA NovoCyte Quanteon flow cytometer and the following gating strategy; Forward Scatter vs. Side Scatter for lymphocytes, cell singlets, live cells by minimal Ghost 510 fluorescence intensity, CD8+ cells, CD34⁺ and CD69⁺ cells.

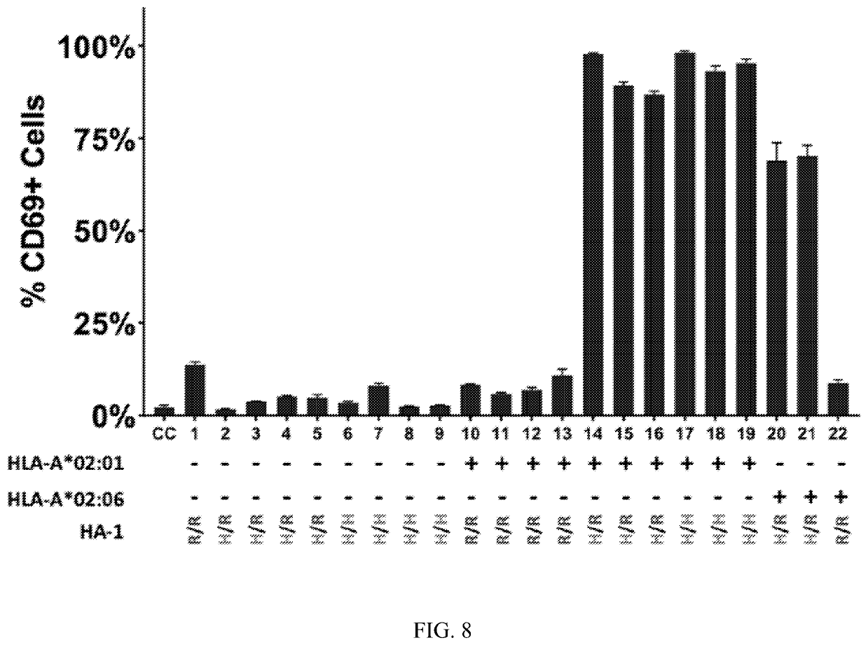

As shown in FIG. 8, upregulation of CD69 only occurred in the presence of target cells expressing both the target miHA antigen (HA-1 “H” epitope) with the cognate HLA allele (HLA-A*02:01 or HLA-A*02:06). The engineered T cells were activated be target cells that were homozygous (H/H) or heterozygous (H/R) for the HA-1 “H” epitope.

Only target cell lines expressing both antigen recognized by the heterologous TCR and the appropriate HLA induced activation of the engineered T cells.

### Example 6. Cytotoxicity of Indication-Specific Cell Lines

The ability of engineered T cells to kill indication-specific target cells was analyzed. The ability of engineered T cells expressing a heterologous anti-HA-1 “H” epitope TCR to target and kill A*02:01⁺/HA-1 H⁺ target cells was evaluated.

THP-1 cells are A*02:01⁺/HA-1 H⁺ (H/R) human monocytic cells derived from an acute monocytic leukemia patient. NALM-6 cells are A*02:01⁺/HA-1 H⁺ (H/R) B cell precursor leukemia cells initiated from an adolescent male. NALM-6 cells are a CD24⁺ xenograft model of acute lymphoblastic leukemia. Non-immunogenic LCL224 cells were included as a control for non-specific killing in the presence of the THP-1 cells. Engineered T cells (expressing or not expressing the heterologous TCR) and target/control cells were cocultured in 96 U-bottom plates overnight (16-18 hours) in an incubator at 37° C. and 5% CO₂. Samples were run in triplicate and analyzed for cell counts by flow cytometry and gating on the CFSE populations.

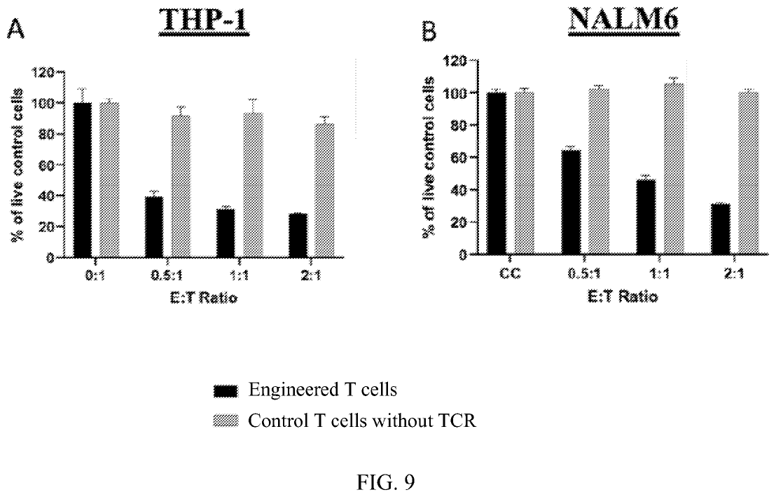

As shown in FIG. 9, the engineered T cells expressing the heterologous TCR effectively killed indication-specific cell lines THP-1 (AML) and NALM6 (B-ALL). T cells not expressing the heterologous TCR did not kill the indication-specific cells.

### Example 7. Expression of Naïve (CD45RA) and Memory (CD45RO) on Engineered T Cells

PBMCs were harvested and engineered T cells expressing a heterologous TCR were prepare as described above using either OKT3 or TRANSACT™ as activator. The engineered T cells were analyzed for CD45 markers prior to incubation with LCL222 target cells (after initial expansion) or after exposure to target cells and re-activation. As shown in Table 6, the engineered T cells primarily had a memory (Tscm or Tcm) phenotype. The engineered T cells made using the described methods maintained a high level of cytotoxicity over time and at least two exposures to target cells (re-stimulation). IC50 values for initial stimulation (day 12) and a second restimulation (on day 32 after a first re-stimulation on day 22) were 0.1556 and 0.11452, respectively.

TABLE 6
Expression of memory T cell markers on engineered T cells.

| Activation | CD45RA⁺/CD45RO⁻ | | CD45RA⁻/CD45RO⁺ | | CD45RA⁺/CD45RO⁺ | |
|---|---|---|---|---|---|---|
| Method | initial | re-activation | initial | re-activation | initial | re-activation |
| OKT3 | 5.14 | 0.04 | 33.75 | 42.18 | 50.77 | 47.01 |
| TRANSACT ™ | 0.45 | 1.84 | 42.66 | 49.52 | 50.1 | 40.03 |

### Example 8. Manufacture of Engineered T Cells Expressing a Heterologous TCR Using a Single Mobilized Apheresis

PBMCs were collected from a donor subject, activated with OKT3 (anti-CD3 monoclonal antibodies), OKT3+anti-CD28 antibodies, or TRANSACT™ (beads containing CD3 and CD28 agonists), and CD8⁺/CD34⁺ cells were selected. Following activation and selection, cells processed for knockout of the endogenous TCR by CRISPR and transduced with a lentivirus encoding a heterologous TCR. Activation was performed on day 0. CD8⁺/CD34⁺ cells were selected, processed for knockout of the endogenous TCR and transduced with lentivirus on day 1. Cells were then expanded for up to 11 days (until day 12). Expanded cells were harvested and cryopreserved prior to transport (shipment to site for administration to a recipient subject).

### Example 9. Treatment of Hematopoietic Malignancy

Engineered T cells expressing a heterologous TCR are administered to a recipient subject suffering from a hematopoietic malignancy in combination with allogeneic hematopoietic stem cell transplant (alloSCT). The heterologous TCR recognizes a miHA antigen expressed in hematopoietic cells of the recipient subject but not expressed by the donor. Donor subjects are screened to identify a related or unrelated donor subject having an HLA 10/10 match to the recipient subject of a related or unrelated donor subject having an HLA 11/11 or HLA 12/12 match to the recipient subject.

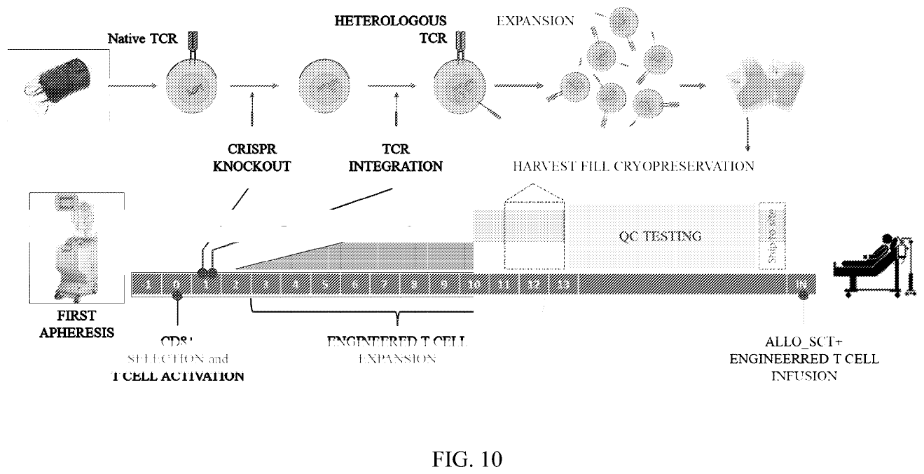

The donor subject is then subjected to a first apheresis procedure to collect PBMCs. The first apheresis procedure is performed without prior mobilization of the donor subject. The first apheresis procedure is performed day −1 (relative to activation of CD8⁺ T cells, see FIG. 10).

Following collection of non-mobilized PBMCs from the donor subject, CD8⁺ T cells are isolated from the PBMCs (day 0). The CD8⁺ T cells are activated overnight (12-24 hours) by incubation with immobilized CD3 and CD28 agonists (TRANSACT™ beads).

After overnight activation, the activate CD8⁺ T cells are genetically modified to knock out expression of the endogenous TRAC and TRBC genes and to insert the nucleic acid sequence encoding the heterologous TCR. Knocking out the endogenous TRAC and TRBC gene is done using CRISPR technology by transfecting the T cells with an RNP complex contains TRAC guide RNA TRBC guide RNA and a Cas9 nuclease. Inserting the nucleic acid sequence encoding the heterologous TCR is done by transducing the T cells with a lentiviral vector containing the nucleic acid sequence encoding the heterologous TCR. Genetically modifying the T cells to form the engineered T cells is performed on day 1.

The engineered T cells are then expanded to provide an effective dose for administration to the recipient subject. T cells are expanded by incubated the engineered T cells in conditions suitable for T cell proliferation for 8-14 days.

Mobilizing agents are administered to the donor subject and the donor subject is subjected to a second apheresis procedure to collect PBMCs. Following collection of mobilized PBMCs from the donor subject, CD34⁺ cells are isolated from the PBMCs to generate an alloSCT graft.

The CD34-selected alloSCT graft and an effective dose of the engineered T cells are both administered to the recipient subject on the same day. If necessary, the recipient subject is administered a conditioning regimen prior to receiving the CD34-selected alloSCT graft and the engineered T cells. The alloSCT graft and the engineered to cells are administered to the recipient subject using methods typical in the art (FIG. 10).

Alternatively, the donor subject may undergo a single mobilized apheresis procedure to provide CD8⁺ cells for manufacture of the engineered T cells and to provide the CD34-selected alloSCT graft.

1. A method of treating a subject having a hematological malignancy comprising:
(a) administering a CD34-selected allogeneic stem cell transplant (alloSCT) graft to the subject, wherein the alloSCT graft is obtained from a graft donor subject that is HLA-matched to the subject; and
(b) administering engineered T cells to the subject, wherein the engineered T cells express a heterologous T cell receptor (TCR) or chimeric antigen receptor (CAR) that recognizes a hematopoietic restricted miHA antigen expressed by the subject;
wherein the engineered T cells are administered to the subject within 72 hours of administering the CD34-selected alloSCT graft to the subject, and
wherein no immunosuppressive agent that targets Graft versus Host Disease (GVHD) is prophylactically administered to the subject to suppress the immune activity of T cells.

2. The method of claim 1 wherein the engineered T cells are administered to the subject within 24 hours of administering the CD34-selected alloSCT graft to the subject.

3. The method of claim 1 or 2, wherein the engineered T cells have been modified to knock out the endogenous TRAC and TRBC genes.

4. The method of any one of claims 1-3, wherein the engineered T cells are derived from a T cell donor subject that is HLA-matched to the subject.

5. The method of any one of claims 1-4, wherein the graft donor subject and the T cell donor subject are the same donor subject.

6. The method of claim 5, wherein the donor subject is selected from the group consisting of: a related donor having an HLA 10/10 match to the subject, an unrelated donor having an HLA 10/10 match to the subject a related donor having an HLA 11/12 match to the subject, a related donor having an HLA 12/12 match to the subject, a unrelated donor having an HLA 11/12 match to the subject, a unrelated donor having an HLA 12/12 match to the subject.

7. The method of claim 6, wherein the donor subject is an HLA 11/12 match to the recipient subject, wherein the donor subject and the subject share a single mismatch at the HLA-DQ or the HLA-DP locus in the graft-versus-host direction.

8. The method of any one of claims 1-7, wherein the subject is administered a conditioning regimen prior to administration of the CD34-selected alloSCT graft.

9. The method of any one of claims 1-8, wherein the hematopoietic restricted miHA antigen comprises an HA-1 epitope or an HA-2 epitope.

10. The method of claim 9, wherein the HA-1 epitope comprises SEQ ID NO: 3.

11. The method of claim 9, wherein the HA-2 epitope comprises SEQ ID NO: 5

12. The method of any one of claims 1-10, wherein the heterologous TCR comprises SEQ ID NO: 8 and SEQ ID NO: 14.

13. The method of claim 12, wherein the heterologous TCR comprises SEQ ID NO: 12 and SEQ ID NO: 18.

14. The method of claim 13, wherein the engineered T cell expresses a nucleic acid sequence encoding SEQ ID NO: 20.

15. The method of any one of claims 1-14, wherein the engineered T cell further expresses an RQR8 peptide.

16. The method of claim 15, wherein the engineered T cells expresses a nucleic acid sequence encoding SEQ ID NO: 22.

17. The method of claim 16, wherein the engineered T cells comprises a nucleic acid sequence comprising SEQ ID NO: 21 or SEQ ID NO: 23.

18. The method of any one of claims 1-17, wherein step (b) further comprises, prior to the administering engineered T cells:
(i) performing an apheresis procedure on the T cell donor subject to collect an apheresis product;
(ii) isolating CD8⁺ T cells from the apheresis product;
(iii) activating the CD8⁺ T cells;
(iv) genetically modifying the CD8^{m} T cells to knock out the endogenous TRAC and TRBC genes;
(v) genetically modifying the CD8⁺ T cells from step (iv) to express the heterologous TCR or CAR thereby generating the engineered T cells; and
(vi) expanding the engineered T cells.

19. The method of claim 18, wherein the number of stem cells in the apheresis product has not been increased by the administration of a mobilizing agent to the T cell donor subject prior to the apheresis procedure.

20. The method of any one of claims 1-19, wherein step (a) further comprises, prior to the administering the CD34-selected alloSCT graft:
(i) administering a mobilizing agent to the graft donor subject;
(ii) performing an apheresis procedure on the graft donor subject to collect an apheresis product; and
(iii) isolating CD34⁺ cells from the apheresis product to generate the CD34-selected alloSCT graft.

21. A method of treating a subject suffering from a hematological malignancy comprising:
(a) generating engineered T cells by genetically modifying CD8⁺ T cells from a donor apheresis or leukapheresis product to knock out the endogenous TRAC and TRBC genes and express a heterologous TCR that recognizes a hematopoietic-restricted miHA antigen expressed by the subject thereby, wherein the donor apheresis or leukapheresis product is obtained from a donor subject that is HLA-matched to the subject but does not express the hematopoietic-restricted miHA antigen; and
(b) administering to the subject the engineered T cells and a CD34-selected alloSCT graft, wherein the CD34-selected alloSCT graft comprises CD34⁺ cells obtained from the donor subject and wherein the engineered T cells are administered to the subject within 72 hours of administering the CD34-selected alloSCT graft to the subject;
wherein no immunosuppressive agent that targets GVHD is prophylactically administered to the recipient subject to suppress the immune activity of T cells.

22. The method of claim 21 wherein the engineered T cells are administered to the subject within 24 hours of administering the CD34-selected alloSCT graft to the subject

23. The method of any one of claims 1-22, wherein subject the hematological malignancy is leukemia, acute lymphoblastic leukemia (ALL), acute myeloid leukemia (AML), myelodysplastic syndrome (MDS), chronic lymphocytic leukemia, chronic myeloid leukemia, chronic myeloid leukemia in blast crisis, chronic myeloid leukemia in accelerated phase, multiple myeloma, or non-Hodgkin's lymphoma.

24. The method of claim 23, wherein the subject has no measurable residual disease (MRD).

25. The method of claim 23, wherein the subject has MRD or morphologically identifiable disease.

26. The method of claim 25, wherein the subject is refractory to at least one prior therapy.

27. The method of claim 26, wherein the at least one prior therapy comprises induction therapy and/or consolidation therapy.

28. The method of any one of claims 25-27, wherein the subject has ≥5% myeloblasts in their bone marrow and/or detectable myeloblasts in their peripheral blood.

29. The method of claim 28, wherein the subject has ≤25% myeloblasts in their bone marrow.

30. The method of any one of claims 23-29, wherein the subject has one or more risk factors or indicators of poor outcome.

31. The method of claim 30, wherein the one or more risk factors or indicators of poor outcome are selected from the group consisting of: a TP53 mutation, a complex karyotype, a typical complex karyotype, an atypical complex karyotype, a monosomal karyotype, a 17p chromosomal abnormality, and a Ph+ chromosomal abnormality.

32. The method of any one of claims 25-31, wherein the subject has AML and up to 25% myeloblasts in bone marrow.

33. The method of any one of claims 23-32, wherein the subject has AML and at least one risk factor selected from the group consisting of: a TP53 mutation, a complex karyotype, a monosomal karyotype, a 17p chromosomal abnormality, a Ph+ chromosomal abnormality, and a MECOM (EVI1) rearrangement.

34. The method of any one of claims 23-33, wherein the subject has AML and detectable RUNX1-RUNX1T1 transcripts, CBFB-MYH11 transcripts, NPM1 mutant transcripts, or FLT3-ITD transcripts in blood or bone marrow.

35. The method of any one of claims 25-31, wherein the subject has ALL and up to 25% myeloblasts in bone marrow.

36. The method of any one of claims 23-31 and 35, wherein the subject has ALL and at least one risk factor selected from the group consisting of: a persistent disease-defining cytogenetic abnormality, a Ph+ chromosomal abnormality, a CRLF2 mutation, an Ikaros deletion, monosomy 7, and a complex karyotype.

37. The method of any one of claims 25-31, wherein the subject has MDS, MRD, and up to 25% myeloblasts in bone marrow.

38. The method of any one of claims 23-31 and 37, wherein the subject has MDS and at least one risk factor selected from the group consisting of: a complex karyotype, a monosomal karyotype, a TP53 mutation, an RAS-pathway mutation, a JAK2 mutation, a RUNX1 mutation, and a ASXL1 mutation.

39. The method of any one of claims 1-38, wherein the subject is in condition for hematopoietic stem cell transplant.

40. The method of any one of claims 1-39, wherein administering the CD34-selected alloSCT graft and the engineered T cells to the subject: lowers the rate of relapse of the hematological malignancy, reduces the severity of relapse of the hematological malignancy, delays recurrence of the hematological malignancy, increases relapse-free survival, reduces toxicity associated with the alloSCT, reduces treatment-related mortality of alloSCT, promotes engraftment of the CD34-selected alloSCT graft, reduces immunologic rejection of the CD34-selected alloSCT graft, reduces GVHD following alloSCT, and/or reduces the risk of GVHD following alloSCT.

41. A method for manufacturing engineered T cells for administration to a subject comprising:
(a) identifying a donor subject that is HLA-matched to the subject or having a donor subject that is HLA-matched to the subject;
(b) collecting an apheresis product from the donor subject or having an apheresis product collected from the donor subject;
(c) selecting CD8⁺ T cells from the apheresis product to form an enriched CD8⁺ T cell population;
(d) activating the CD8⁺ T cells in the enriched CD8⁺ T cell population;
(e) genetically modifying the CD8⁺ T cells to express a nucleic acid encoding a heterologous TCR or chimeric antigen receptor (CAR) thereby the generating the engineered T cells, wherein the engineered T cells express the heterologous TCR or CAR; and
(f) expanding the engineered T cells.

42. The method of claim 40 wherein the donor subject is selected from the group consisting of: a related donor having an HLA 10/10 match to the subject, an unrelated donor having an HLA 10/10 match to the subject, a related donor having an HLA 11/12 match to the subject, a related donor having an HLA 12/12 match to the subject, a unrelated donor having an HLA 11/12 match to the subject, a unrelated donor having an HLA 12/12 match to the subject.

43. The method of claim 42, wherein the donor subject is an HLA 11/12 match to the recipient subject, wherein the donor subject and the subject share a single mismatch at the HLA-DQ or the HLA-DP locus in the graft-versus-host direction.

44. The method of any one of claim 41-43, wherein the apheresis product is collected by apheresis or leukapheresis without prior administration of a mobilizing agent to the donor subject.

45. The method of any one of claims 40-44, wherein activating the CD8⁺ T cells comprises contacting the CD8⁺ T cells with:
(i) a soluble anti-CD3 antibody in the absence of a CD28 agonist;
(ii) a CD3 agonist and a CD28 agonist; or
(iii) immobilized CD3 and CD28 agonists.

46. The method of claim 45, wherein CD8⁺ T cells are activated for about 12 to about 24 hours.

47. The method of any one of claims 41-46, wherein the method further comprises knocking out the endogenous T cell receptor (TCR) in the activated CD8⁺ T cells after step (d) and before step (e).

48. The method of claim 47, wherein knocking out the endogenous T cell receptor comprises transfecting the CD8⁺ T cells with an Cas9/guide RNA pre-formulated ribonucleoprotein complex targeting the TRAC and TRBC genes, wherein the pre-formulated ribonucleoprotein complex a guide RNA comprising SEQ ID NO: 1 and a guide RNA comprises SEQ ID NO: 2.

49. The method of any one of claims 41-48, wherein genetically modifying the CD8⁺ T cells to express the heterologous TCR or CAR comprises introducing a vector encoding the heterologous TCR or CAR into the CD8⁺ T cells.

50. The method of claim 49, wherein introducing the vector encoding the heterologous TCR or CAR into the CD8⁺ T cells comprises transducing the CD8⁺ T cells with a lentiviral vector comprises a nucleic acid sequence encoding the heterologous TCR or CAR.

51. The method of claim 50, wherein the engineered T cell contains 5 or fewer copies of a nucleic acid encoding the heterologous TCR or CAR.

52. The method of any one of claims 41-51, wherein the heterologous TCR comprises a TCR having affinity for a hematopoietic restricted miHA antigen expressed by the subject.

53. The method of claim 52, wherein the hematopoietic restricted miHA antigen comprises an HA-1 epitope or an HA-2 epitope.

54. The method of claim 53, wherein the HA-1 epitope comprises SEQ ID NO: 3.

55. The method of claim 53, wherein the HA-2 epitope comprises SEQ ID NO: 5

56. The method of any one of claims 41-54, wherein the heterologous TCR comprises SEQ ID NO: 8 and SEQ ID NO: 14.

57. The method of claim 56, wherein the heterologous TCR comprises SEQ ID NO: 12 and SEQ ID NO: 18.

58. The method of claim 57, wherein the engineered T cell expresses a nucleic acid sequence encoding SEQ ID NO: 20.

59. The method of any one of claims 41-58, wherein the engineered T cell further expresses an RQR8 peptide.

60. The method of claim 59, wherein the engineered T cells expresses a nucleic acid sequence encoding SEQ ID NO: 22.

61. The method of claim 60, wherein the engineered T cells comprises a nucleic acid sequence comprising SEQ ID NO: 21 or SEQ ID NO: 23.

62. The method of any one of claims 41-61, wherein steps (c), (d), and (e) are performed 24-48 hours after step (b).

63. The method of any one of claims 41-62, wherein expanding the engineered T cells comprises incubating the engineered T cells in conditions suitable for growth for about 7 to about 14 days.

64. The method of any one or claims 41-63, wherein at least 10% of engineered T cells have a stem-cell like phenotype.

65. The method of any one or claims 41-64, wherein the engineered T cells kill cells expressing an antigen recognized by the heterologous TCR or CAR at an Effector:Target ratio less than 0.5:1.

66. The method of claim 65, wherein the engineered T cells maintain at least 50% of their cytolytic potency over a period of a least 20 days.

67. The method of any one of claims 41-66, further comprising cryopreserving the engineered T cells after step (f).

68. The method of claim 67, wherein the engineered T cells are cryopreserved within about 9, 10, 11, 12, 13, 14, or 15 days of harvesting the apheresis product from the donor subject.

69. The method of claim 67 or 68, wherein the cryopreserved engineered T cells, when thawed, kill cells expressing an antigen recognized by the heterologous TCR or CAR at an Effector:Target ratio of less than 0.5:1.

70. The method of any one of claims 67-69, wherein the cryopreserved engineered T cells, when thawed, maintain at least 50% of their cytolytic potency for at least 20 days.

71. The method of any one of claims 41-70, wherein the engineered T cells increase graft versus leukemia effect in a subject treated with allogeneic hematopoietic stem cell transplantation (alloSCT) and/or reduce the risk of graft versus host disease in a subject treated with alloSCT.

72. An ex vivo engineered T cell made by the method of any of one claims 41-71.

73. A method of treating a subject having a hematological malignancy comprising: administering the engineered T cells of claim 72 to the subject.

74. The method of claim 73, wherein the engineered T cells are administered to the subject in combination with alloSCT.

75. The method of claim 74, wherein the alloSCT comprises CD34-selected alloSCT.

76. The method of claim 75, wherein the engineered T cells are administered to the subject on the same day as the CD34-selected alloSCT, wherein no immunosuppressive agent that targets GVHD is prophylactically administered to the recipient subject to suppress the immune activity of T cells.

77. The method of any one of claims 73-76, wherein the hematological malignancy comprises: acute myeloid leukemia, myelodysplastic syndrome, or acute lymphoblastic leukemia.

78. A nucleic acid comprising a sequence encoding SEQ ID NO: 20 or SEQ ID NO: 22.

79. The nucleic acid of claim 78, wherein the nucleic acid comprises:
(a) a sequence having at least 75% identity to SEQ ID NO: 19, SEQ ID NO: 21, SEQ ID NO: 23, or SEQ ID NO: 24; or
(b) a sequence comprising SEQ ID NO: 19, SEQ ID NO: 21, SEQ ID NO: 23, or SEQ ID NO: 24.

80. A lentiviral vector comprising:
(a) a nucleic acid sequence encoding SEQ ID NO: 20 or SEQ ID NO: 22;
(b) a nucleic acid sequence having at least 75% identity to SEQ ID NO: 19, SEQ ID NO: 21, or SEQ ID NO: 23 and encoding the amino acid sequence of SEQ ID NO: 20 or SEQ ID NO: 22; or
(c) a nucleic acid sequence comprising SEQ ID NO: 19, SEQ ID NO: 21, or SEQ ID NO: 23.

81. An engineered T cell comprising:
(a) a nucleic acid sequence encoding SEQ ID NO: 20 or SEQ ID NO: 22;
(b) a nucleic acid sequence having at least 75% identity to SEQ ID NO: 19, SEQ ID NO: 21, or SEQ ID NO: 23 and encoding the amino acid sequence of SEQ ID NO: 20 or SEQ ID NO: 22; or
(c) a nucleic acid sequence comprising SEQ ID NO: 19, SEQ ID NO: 21, or SEQ ID NO: 23.
